(19) United States
(12) Patent Application Publication
(10) Pub. No.: US 2022/0411378 A1
(43) Pub. Date: Dec. 29, 2022

(54) SMALL MOLECULES POLYMERASE INHIBITORS

(71) Applicants: GEORGIA STATE UNIVERSITY RESEARCH FOUNDATION, INC., Atlanta, GA (US); ICAHN SCHOOL OF MEDICINE AT MOUNT SINAI, New York, NY (US)

(72) Inventors: Robert COX, Decatur, GA (US); Richard PLEMPER, Decatur, GA (US); Benhur LEE, New York, NY (US)

(21) Appl. No.: 17/776,934

(22) PCT Filed: Sep. 15, 2020
(86) PCT No.: PCT/US2020/050887

§ 371 Date: May 13, 2022

Related U.S. Application Data
(60) Provisional application No. 62/935,896, filed on Nov. 15, 2019.

(51) Int. Cl. C07D 217/02; A61P 31/14; C07D 239/88; C07D 217/14

(52) U.S. Cl. CPC C07D 217/02; A61P 31/14; C07D 239/88; C07D 217/14

(57) ABSTRACT

Disclosed herein are compounds using for inhibiting RNA polymerase, especially viral RNA polymerase. The compounds are effective for the treatment of viruses in Paramyxoviridae family, especially measles and human parainfluenza virus.

### CROSS-REFERENCE TO RELATED APPLICATION

This application claims the benefit of U.S. Provisional Application 62/935,896, filed on Nov. 15, 2019, the contents of which are hereby incorporated in its entirety.

### STATEMENT OF GOVERNMENT SUPPORT

This invention was made with government support under Grant No. A1071002, awarded by National Institute of Allergy and Infectious Diseases. The government has certain rights in the invention.

### FIELD OF THE INVENTION

The invention is directed to compounds that inhibit RNA-polymerases, including viral RNA-polymerases. The compounds are especially useful for treating various paramyxoviruses, including human parainfluenza and measles.

### BACKGROUND

The Paramyxoviridae family of negative-sense RNA viruses contains the etiologic agents of major human diseases, such as the human parainfluenza viruses (HPIVs; types 1-4) and measles virus (MeV). HPIVs are a leading cause of hospitalization for respiratory illness in young children and responsible for over 70,000 hospitalizations annually in the United States. In developing countries, HPIV infections in children are associated with much higher incidences of mortality, with estimates exceeding 112,000 deaths per year Immunocompromised individuals, such as transplant recipients, are particularly susceptible to severe and often fatal HPIV disease, with mortality rates ranging up to 75%. Neither vaccines nor effective antivirals are available to prevent or treat HPIV infections, making development of an effective antiviral an urgent clinical need.

HPIV type-3 (HPIV-3), estimated to cause over 3 million cases of medically-attended acute respiratory infections in the US each year, is the most prevalent source of HPIV disease. Antiviral therapeutics targeting viral polymerases have been successfully employed to combat a variety of viral threats, from HIV to influenza. However, the use of polymerase inhibitors, in particular allosteric small-molecule inhibitors, is typically limited to a single viral species or genus.

There remains a need for improved polymerase inhibitors against all types of viruses. There remains a need for small-molecule inhibitors that are effective against multiple viral species. There remains a need for improved small-molecule polymerase inhibitors effective against Paramyxoviridae family viruses.

### SUMMARY

The details of one or more embodiments are set forth in the descriptions below. Other features, objects, and advantages will be apparent from the description and from the claims.

### BRIEF DESCRIPTION OF THE FIGURES

FIG. 1A-1G. Identification and initial characterization of GHP-88309. FIG. 1A, Results of primary HPIV3 HTS screen and counterscreening of hit candidates (enlarged area). FIG. 1B, GHP-88309 chemical structure. FIG. 1C, Bioactivity and toxicity profiling of resynthesized GHP-88309. FIG. 1D, GHP-88309 has a broadened activity spectrum covering paramyxoviruses of two genera (left panel). Table summarizes EC₅₀ and EC₉₀ concentrations and SI values against different viral targets (right panel). FIG. 1E, Antiviral activity of GHP-88309 against clinical isolates of HPIV3 on disease-relevant primary human airway cells. Effects of GHP-88309 on cell proliferation was assessed on HBTECs (CC₅₀=1 mM). FIG. 1F, MeV ToA studies. Known MeV fusion (AS-48) and MeV polymerase (ERDRP-0519) inhibitors and a host-directed polymerase inhibitor (JMN3-003) are included for reference. FIG. 1G, Inhibitory activity of GHP-88309 against paramyxovirus and pneumovirus minigenome reporter systems. In (FIG. 1C-1G), symbols represent biological repeats and lines connect data means.

FIG. 2A-2M. GHP-88309 targets a conserved microdomain in the L protein, inhibiting de novo RNA synthesis. FIG. 2A, Schematic overview and summary of confirmed resistance mutations identified from adaptation of three different target viruses (HPIV3, MeV, SeV as red, blue, and orange tick marks, respectively) to GHP-88309. FIG. 2A-2C, Color-coded mapping of all confirmed resistance mutations on a model of VSV L (PDB:5A22) (FIG. 2B). Hot-spots from all viral targets line a microdomain between RdRP and capping domains at the entrance to the template channel (HPIV3, red spheres; SeV, orange spheres; MeV, blue spheres) The GDN active site for the RdRP domain is shown as purple spheres. (FIG. 2C). d, Mapping of all confirmed resistance mutations onto a homology model of the HPIV3 L resistance site. FIG. 2E, In silico docking of GHP-88309 into homology models of MeV (blue), SeV (orange), and HPIV3 (magenta) proposes a conserved binding pose between GHP-88309 and the L microdomain. An overlay of the top-scoring docking poses is shown in front of the HPIV3 L microdomain, colored by RdRP (cyan) and capping domain (green). Resistance sites are shown in red and labeled. FIG. 2F, BLI to test direct interaction between GHP-88309 and purified MeV L, and MeV L harboring selected resistance mutations. FIG. 2G, Summary of K_{D} values of all standard and resistant MeV L constructs tested in (FIG. 2F). FIG. 2H-2J, Inhibition of primary viral mRNA transcription by GHP-88309 measured 2 (FIG. 2H; HPIV3), 4 (FIG. 2I; HPIV3), or 4 (FIG. 2J; MeV) hours after infection. FIG. 2K, Effect of GHP-88309 on relative MeV Le RNA concentration, measured 3 hours after infection of cells. FIG. 2L-2M, Autoradiograms after polyacrylamide gel fractionation of in vitro MeV RdRP assay products. Reactions contained purified MeV P-L proteins, synthetic RNA templates, and a ³²P-GTP tracer. GHP-883089 in escalating concentrations does not block 3′ RNA extension after back-priming (FIG. 2K), but the compound inhibits de novo initiation of RNA synthesis at the promoter (FIG. 2L). A reaction with inactive L_{N774A} was used for specificity control. In (FIG. 2H-K), symbols represent biological repeats, columns show means±SD. Significance was tested by one-way ANOVA with Dunnett's multiple comparisons post-hoc test. Individual P values are shown.

FIG. 3A-3E. GHP-88309 is efficacious in well-differentiated human airway epithelium cultures grown at air-liquid interface (3D-ALI-HBTEC). FIG. 3A, Effect of increasing concentrations of GHP-88309 on trans-epithelial electrical resistance (TEER) in the 3D-ALI-HBTEC culture. FIG. 3B, Confocal microscopy examining tight junction integrity (α ZO-1) after incubation of 3D-ALI-HBTEC cultures with 640 μM GHP-88309. FIG. 3C-3D, Antiviral potency of GHP-88309 against HPIV3 strain JS and two clinical HPIV3 isolates (10L3 and 9R4) (FIG. 3C) and clinical HPIV1 isolate 5F6 (FIG. 3D) in 3D-ALI-HBTEC cultures. Compound was added to the basolateral chambers at the specified concentration ranges. FIG. 3E, Confocal microscopy of HPIV3-JS-infected 3D-ALI-HBTEC cultures. Stained are ciliated cells (αβ-tubulin), HPIV3 antigens (α HPIV3), and nuclei (DAPI). GHP-88309 added to the basolateral chamber at 10 μM is sterilizing. In (FIG. 3A, FIG. 3C-3D), symbols represent biological repeats, lines connect mean values. Active concentrations of GHP-88309 were determined through 4-parameter variable slope regression modeling (FIG. 3C-D).

FIG. 4A-4E. Pharmacokinetic (PK) characterization of GHP-88309. FIG. 4A, Stability of GHP-88309 after incubation with mouse or human plasma, or mouse or human liver microsomes, respectively. Half-life (t_{1/2}) of GHP-88309 was estimated to be >15 hours in all conditions tested. Positive controls were procaine, benfluorex, and verapamil FIG. 4B, Single-dose oral vs i.v. PK study of GHP-88309 in mice. Selected calculated PK parameters and oral bioavailability are shown. FIG. 4C, Tissue distribution of GHP-88309 in mice. Specified tissue were extracted 90 minutes after a single oral dose at 150 mg/kg. d, Blood drug levels after multi-dose administration of GHP-88309 to mice at 150 mg/kg, given orally b.i.d. for a total of 4.5 days. Blood was collected at peak (Cₘₐₓ; 0.5 hours after dosing; blue) and trough (11.5 hours after dosing; red). FIG. 4E, Comparison of mouse blood PK curves after single and multi oral dosing of GHP-88309 (150 mg/kg). Multi-dose animals from (FIG. 4D), samples on day 5 after initiation of dosing. Statistical analysis through two-way ANOVA with Dunnett's multiple comparisons post-hoc test. Throughout, symbols represent biological repeats, lines connect mean values (FIGS. 4A, 4B, 4D, 4E). Columns represent data means±SD (FIG. 4C).

FIG. 5A-5H. GHP-88309 is efficacious in a SeV mouse surrogate assay of HPIV infection. FIG. 5A-5E, Oral efficacy of GHP-88309 after b.i.d. dosing of mice infected intranasally with 1.5×10⁵ TCID₅₀ units SeV at 150 mg/kg. Treatment was initiated two hours before (prophylactic) or 48 hours after (therapeutic) infection. Clinical signs (body weight (FIG. 5A) and temperature (FIG. 5B)) were monitored daily. Survival (FIG. 5E) was calculated 9 days after infection. On days 3, 6, and 9 after infection (days pI), virus burden in trachea (FIG. 5C) and lungs (FIG. 5D) were determined. Symbols represent individual animals, lines connect medians. Statistical analysis through two-way ANOVA with Dunnett's multiple comparisons post-hoc test. FIG. 5F, Representative lung sections after H&E staining of mock or SeV-infected mice, treated with GHP-88309 as in (FIG. 5A-5E). Lungs were extracted 6 days after infection. Scale bars represent 100 μm. Black arrows point to sites of immune infiltration. g, Histopathology scores of lung sections as shown in (FIG. 5E). Per condition, >16 distinct sections of 3 animals were analyzed. For mock infected vehicle treated images, 6 distinct sections were used from one mouse. Symbols represent individual histopathology scores, columns show means±SD. Statistical analysis through one-way ANOVA with Tukey's multiple comparisons post-hoc test. FIG. 5H, Heat maps of relative mRNA amounts of selected host genes associated with antiviral response pathways in mice of the prophylactic and therapeutic treatment groups, all shown in relation to vehicle-treated mice on days 3 and 6 after infection, respectively. Color-coding denounces significantly higher (red), lower (blue), or unchanged (grey) mRNA amounts relative to the vehicle-treated animals. Statistical analysis through one-way ANOVA with Dunnett's multiple comparisons post-hoc test.

FIG. 6A-6K. Effect of GHP-88309 treatment on adaptive immunity and rechallenge, and correlation between resistance and viral pathogenesis. FIG. 6A, Schematic of long-term survival and rechallenge study. Animals were treated therapeutically with GHP-88309 at 150 mg/kg dose concentration, administered b.i.d. b-c, Daily monitoring of clinical signs (body weight (FIG. 6B) and temperature (FIG. 6C)) after initial challenge with 1.5×10⁵ TCID₅₀ units SeV administered intranasally. FIG. 6D, Survival curve of SeV-infected mice after primary infection and therapeutic (+48 hours after infection) GHP-88309 treatment. FIG. 6E, Assessment of neutralizing antibodies directed against SeV of mice before treatment (antiserum (−2 d)), before rechallenge (antiserum (21 days)), and after rechallenge (antiserum (48 days)). Neutralizing effects of FBS and culture media (media) were tested for control. FIG. 6F-6G, Intranasal rechallenge of recoverees from (b) with a 1.5×10⁵ TCID₅₀ of SeV on day 28 after the primary infection. Monitored were clinical signs (body weight (FIG. 6E) and temperature (FIG. 6F)). For reference, a new group of naïve mice were infected equally in parallel. h, Survival curves of SeV-infected mice from (FIG. 6F-6G). FIG. 6I, Growth curves of standard recSeV and recSeVs with confirmed GHP-88309 resistance mutations in cultured cells. FIG. 6J-6K, Effect of resistance mutations on SeV pathogenesis in mice. Daily monitoring of body weight (FIG. 6I) and overall survival (FIG. 6J) after intranasal infection with 1.5×10⁵ TCID₅₀ of standard recSeV or three distinct recSeVs with the specified signature resistance mutations. Throughout, symbols represent biological repeats, lines connect means (FIGS. 6B-6C; 6F-6G; 6J) or medians (FIG. 6I). Error bars in (FIG. 6E) show SD. Statistical analysis through two-way ANOVA and Dunnett's multiple comparisons post-hoc test (FIGS. 6B-6C, 6E-6F, 6I) or one-way ANOVA and Dunnett's multiple comparisons post-hoc test (FIG. 6E). Statistical differences in time-to-death were explored through log-rank (Mantel-Cox) test.

FIG. 7A-7K. (FIG. 7A-7H) Anti-HPIV3 HTS hit identification and validation. FIG. 7A, Schematic of genome organization of recHPIV3-JS-NanoLuc. FIG. 7B, Validation of automated HTS protocol in 384-well format, using recHPIV3-JS-NanoLuc as detection agent. Three 384-well assay validation plates featuring alternating columns containing vehicle (Max) or the broad-spectrum host-directed inhibitor JMN3-003 in intermediate (0.5×EC₅₀; Med) or sterilizing (10×EC₅₀; Min) concentrations. On each validation plate, control columns were arranged in different order. Average signals, signal-to-background (S/B) and Z′ scores for each reference plate were calculated and are listed in the table. FIG. 7C, Dose-response antiviral activity and cytotoxicity tests with sourced hit candidates GHP-88309 and GHP-64627. FIG. 7D, Comparison of antiviral activity of commercially sourced and resynthesized GHP-88309. FIG. 7E, Effect of GHP-88309 on cell proliferation of different immortalized cell lines. No toxicity was detectable in the concentration range tested (up to 100 μM). FIG. 7F) Effect of GHP-88309 on metabolic activity of primary human BTECs. Cytotoxicity was assessed by measuring COX-1 protein levels relative to vehicle treated HBTECs. Effects of compound incubation was assessed in a dose-response format with the highest concentration equal to 1000 μM. Mitochondrial toxicity was tested in parallel after by measuring SDH-A protein levels relative to vehicle treated HBTECs. Protein levels were measured after 72 hour incubation with GHP-88309. FIG. 7G, Antiviral activity of GHP-88309 determined under different conditions (after infection at high MOI; at different media pH; in the presence of BSA). No significant differences were noted (P=0.329). Statistical analysis through the extra sum-of-squares F test. FIG. 7H, HPIV3 ToA studies. GHP-88309 was added at the specified time points after infection at 20 μM. The host-directed polymerase inhibitor JMN3-003 was included for reference. In (FIG. 7C-7H), symbols represent biological repeats, lines are derived from 4-parameter variable slope regression modeling (FIG. 7C-7G) or connect data means (FIG. 7H). Where applicable, active (EC₅₀) and cytotoxic (CC₅₀) concentrations are shown with 95% confidence intervals (CIs) in parentheses. (FIG. 7I-7K) GHP-88309 hit validation. (FIG. 7I-7K) Efficacy of GHP-88309 against different clinical isolates of HPIV3 (10L3 (KY973583), 9R4 (KY674929), 3-1, 3-2, and 3-3) (FIG. 7I), MeV (FIG. 7J), and HPIV1 (4C5, 2D4 (MF554715.1), 5M6 (MF554714.1), and 5F6) (FIG. 7K). Progeny virus yields from dose-response tests were determined through TCID50 titration. GHP-88309 consistently inhibited all target virus strains with low-micromolar to submicromolar potency; EC50 concentrations are shown.

FIG. 8A-8L. (FIG. 8A-8F) Characterization of viral resistance mutations to GHP-88309. FIG. 8A, All candidate resistance mutations emerging from adaptation of HPIV3 and two engineered combinations thereof were rebuilt and tested in an HPIV minigenome assay of RdRP activity in the presence of GHP-88309. FIG. 8B, Mutations from (FIG. 8A) that were rebuilt in recHPIV3-JS-NanoLuc and tested against GHP-88309 in reporter dose-response assays. FIG. 8C, Candidate mutations emerging from MeV adaptation were rebuilt and tested in an MeV minigenome assay of RdRP activity. FIG. 8D, All candidate resistance mutations emerging from SeV adaptation were rebuilt in recSeV and tested against the resulting recSeVs in virus yield-based dose-response assays. FIG. 8E, SDS-PAGE of purified MeV P-L complexes used in BLI experiments. Samples were fractioned on 7.5% gels, followed by Coomassie blue staining FIG. 8F, NiV minigenome assays to test inhibitory activity of GHP-88309 against standard NiV RdRP and NiV RdRP harboring an L_{H1165Y} point mutation. In (FIGS. 8A-8F), symbols represent biological repeats, lines are derived from 4-parameter variable slope regression modeling, and active concentrations (EC₅₀ and EC₉₀ if applicable) are shown with 95% CIs. Yellow highlights denote experimentally confirmed resistance mutations. (FIG. 8G-8L) GHP-88309-016 target mapping through photoaffinity labeling. FIG. 8G, Structure of GHP-88309-016. FIG. 8H, GHP-88309-016 is bioactive, potently inhibiting MeV and HPIV3 replication without appreciable cytotoxicity (n=3). EC50 values from 4-parameter variable slope regression models are shown with 95% CIs. FIG. 8I, 2D-schematic of the MeV L protein with locations of known resistance mutations (top) and peptides identified by photoaffinity labeling (bottom; black bars). Cyan, green, yellow, orange, and red depict the RdRP, capping, connector, MTase, and C-terminal domains. FIG. 8J, Homology model of MeV L showing the locations of the peptides crosslinked to GHP-88309-016 (black spheres). Confirmed GHP-88309 resistance sites are shown in red. Peptides 1 and 2 are located on the exterior of the polymerase. FIG. 8K, Only residues of peptide 3 (black) are exposed to the interior channels of the polymerase in proximity of the resistance sites (red). The homology model was based on the coordinates reported for HPIV5 L (PDB: 6v85). (FIG. 8L) Steady-state analyses of BLI binding saturation and sensitization of NiV L to GHP-88309. Concentration-dependent steady-state BLI sensor response signals were plotted for the different MeV L1708 samples (standard (WT) or carrying resistance mutations as indicated) to probe whether saturation of binding was reached.

FIG. 9A-9N. (FIG. 9A-9I) In silico docking of GHP-88309. FIGS. 9A-9I, GHP-88309 was docked into homology models of HPIV3 (FIGS. 9A-9C), MeV (FIGS. 9D-9F), and SeV (FIGS. 9G-9I) L proteins. 2D diagrams of top-scoring ligand interactions (FIGS. 9A, 9D, and 9G) were generated with MOE and predict a conserved mode of interaction between GHP-88309 and the L protein target. Ribbon (FIGS. 9B, 9E, and 9H) and surface (FIGS. 9C, 9F, and 9I) representations of the predicted GHP-88309 docking poses, the ligand is shown as stick model. The viral capping and RdRP domains are colored green and blue, respectively, confirmed resistance sites are in red and labeled. Numbers denote predicted free energy for each docking pose. (FIG. 9J-9N) In silico docking of GHP-88309. FIG. 9J, Ribbon representation of the MeV L internal channel, showing distances between confirmed resistance sites (red) and the nearest residue in photoaffinity labeled peptide 3 (D993; black). Polymerase domain color-coding as in FIG. 2A. FIG. 9K, Docking of GHP-88309 (blue sticks) and GHP-88309-016 (yellow sticks) into the MeV L model, using D993 and confirmed resistance hot-spots as target site guides. The topscoring pose is conserved between GHP-88309 and GHP-88309-016, and positions the between capping and RdRP domains. The aryl-azide moiety is located approximately 7.8 Å from residue D993 in peptide 3. Numbers denote predicted free energy associated with this docking pose. FIG. 9L, 2D-diagram of predicted top-scoring ligand interaction generated with MOE. Predicted are hydrogen bond interactions between the isoquinoline ring of GHP-88309 and residues Y942 and R865. The benzamide moiety is posited between residues Q1007 and R1011, thus overall linking RdRP and capping domains. FIG. 9M-9N, Application of the equivalent in silico docking approach as in (FIG. 9K) to NiV (FIG. 9M) and RSV L (FIG. 9N). Top scoring poses in NiV (pink sticks) and RSV (white sticks) L are distinct from that in MeV L (superimposed as blue sticks). Known resistance mutations are colored red (a-b, d, e). NiV homology model is based on HPIV5 L (PDB: 6v85); RSV L is native (PDB: 6pzk).

FIG. 10A-10J. (FIG. 10A-10D) Effect of GHP-88309 treatment on MeV primary transcription. FIG. 10A, Relative MeV antigenome levels, determined through qRT-PCR were from RNA extracts shown in (FIG. 2I). FIG. 10B-10D, Effect of GHP-88309 on primary transcription of viral P (FIG. 10B) and L (FIG. 10C) genes. RNA was extracted from MeV-infected cells 3 hours after virus addition. d, Relative MeV antigenome concentrations in RNA extracts from (FIG. 10B-10C). At this early time after infection, genome replication has not been initiated yet. (FIG. 10E-10J) Effect of GHP-88309 treatment on HPIV3 and MeV primary transcription. FIG. 10E-10G, GHP-88309 significantly reduced levels of MeV P (FIG. 10E), H (FIG. 10F), and L (FIG. 10G) mRNA transcripts at 12 hours after infection. FIG. 10H, GHP-88309 significantly reduced the synthesis of HPIV3 Le RNA 2 hours after infection. FIG. 10I-10J, GHP-88309 did not significantly alter the HPIV3 (e; 2 hours after infection) and MeV (f; 4 hours after infection) primary mRNA transcription gradients. Values represent relative changes compared to vehicle treated samples (a-d) or relative changes compared to HPIV3 N (FIG. 10I) or MeV P (FIG. 10J) mRNA levels. Experiments were conducted in at least three biological repeats, determined in duplicate each. Symbols represent biological repeats, columns show means±SD. Statistical analysis with one-way ANOVA and Dunnett's multiple comparisons post-hoc test (two sided).

FIG. 11A-11D. Preparation and optimization of the MeV in vitro RdRP assay. FIGS. 11A, and 11B, SDS-PAGEs of MeV P-L (FIG. 11A) and MeV P-L_{N774A} (FIG. 11B) protein preparations, purified on Ni-NTA resin. Aliquots of eluted proteins used in biochemical RdRP assays were fractioned on 7.5% gels, followed by visualization through Coomassie blue staining.

FIGS. 11C-11D, Optimization of the MeV in vitro RdRP assay. Autoradiograms of in vitro synthesis products after polyacrylamide gel fractionation; labeling of products through ³²P-GTP tracer. The sequence of the synthetic RNA template is shown. A range of MgCl₂ and MgCl₂ concentrations were explored to identify nature and concentration of the bivalent cation cofactor associated with maximal in vitro polymerase activity.

FIG. 12A-12E. Different synthetic RNA templates used in the MeV in vitro RdRP assay. FIGS. 12A-12B, RSV-derived RNA template (FIG. 12A) that is capable of back-priming depicted in (FIG. 12B). FIG. 12C, MeV-derived RNA template. FIGS. 12D-12E, Results of the MeV RdRP assay with the RSV (FIG. 12D) and MeV (FIG. 12E) derived templates. Only the RSV-derived template back-primes, resulting in assessment of polymerase initiation at the promoter and 3′ extension after back priming, whereas the MeV-derived template tests exclusively polymerase initiation at the promoter.

FIG. 13A-13B Individual measurements of the effect of infection and GHP-88309 treatment on host innate immune response activation as summarized in FIG. 5G. FIG. 13A, Relative expression of genes involved in innate host antiviral response pathways, determined three, six, and nine days after SeV infection of mice through qRT-PCR. Results are shown for vehicle treated, prophylactically treated (2 hours before infection), and therapeutically treated (48 hours after infection) mice, all normalized to mock-infected and untreated reference animals. Symbols represent measurements from individual mice (n=3 in all groups). Statistical analysis with two-way ANOVA and Tukey's multiple comparison post-hoc test. FIG. 13B, Relative induction of type-1 IFN and interferon stimulated genes in undifferentiated HBTECs after HPIV3 infection and treatment with 6 μM GHP-88309. Cells were mock infected and exposed to GHP-88309 (treated mock inf), HPIV3 infected and vehicle-treated (vehicle), or HPIV3 infected and GHP-88309 treated starting 2 hours before infection (prophylactic) or 6 hours after infection (therapeutic). All results are expressed relative to untreated, mock-infected cells.

FIG. 14 is a synthetic scheme of GHP-88309-003 and GHP-88309-004

FIG. 15 is a scheme of the introduction of the propargyl group after boronic acid coupling reaction towards synthesis of GHP-88309-010.

FIG. 16 is a synthetic scheme of GHP-88309-10 and GHP-88309-015 through ester conversion through the corresponding amides.

FIG. 17 shows the GHP-88309 SAR development. Chemical elaboration and bioactivity testing of the GHP-88309 scaffold against HPIV3 and MeV reporter viruses. EC₅₀ and EC₉₀ concentration were calculated through 4-parameter variable slope regression modeling of dose-response data. Cytotoxicity was assessed through PrestoBlue assay (n=3).

FIG. 18 shows the mapping of RSV L capping blocker and AZ-27 resistance hotspots. Analogous positions of resistance mutations against the RSV L capping inhibitor compound C (E1269D and I1381S; magenta spheres) and initiation blocker AZ-27 (Y1631; orange spheres) are projected onto an HPIV3 L homology model when locatable. Resistance mutations against the capping inhibitor are proximal to conserved PRNTase motifs (blue spheres) and distal to GHP-88309 resistance mutations (red spheres). The RdRP, connector, and capping domains are color-coded as in FIG. 2A. The HPIV3 L homology model is based on the coordinates reported for HPIV5 L (PDB: 6v85).

FIG. 19 is a table showing HPIV3 HTS Counterscreens. Overview of direct and orthogonal counterscreens and applied to hit identification after primary HTS. Requested cut-offs for further advance of a hit candidate and performance of GHP-88309 and GHP-64627 are shown.

FIG. 20 is a table showing photoaffinity labeling results. Three distinct peptides were detected by LC-MS/MS that contained additional mass corresponding to that of GHP-88309-016. Predicted localization of crosslinks, coverage, and the peptide spectrum match (PSM) score calculated in pFind are shown.

FIG. 21 is a table showing in vivo adaptation of recSeV to GHP-88309. Serial passaging of recSeV in mice in the presence of 50 mg/kg body weight GHP-88309.

FIG. 22 shows paramyxovirus L sequence alignments. Covered are confirmed GHP-88309 resistance sites and positions of GHP-88309 resistance mutations are highlighted (boxes). A single residue critical for susceptibility to GHP-88309 is not conserved in NiV L at position 1156. Genera and subfamilies are color-coded (respirovirus, green; morbillivirus, blue; henipavirus, orange; Ferlavirus, yellow; avulavirinae, pink; aquapramyxovirus, light green; rubulavirinae, light blue).

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. For example, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps.

Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited.

“Exemplary” means “an example of” and is not intended to convey an indication of a preferred or ideal embodiment. “Such as” is not used in a restrictive sense, but for explanatory purposes.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

Administration of the therapeutic agents can be carried out at dosages and for periods of time effective for treatment of a subject. In some embodiments, the subject is a human.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cycloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. A cycloalkyl group which contains no double or triple carbon-carbon bonds is designated a saturated cycloalkyl group, whereas a cycloalkyl group having one or more such bonds (yet is still not aromatic) is designated an unsaturated cycloalkyl group. Unless specified to the contrary, the term cycloalkyl embraces both saturated and unsaturated, non-aromatic, ring systems.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H˜ 1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. In a specific example, groups that are said to be substituted are substituted with a protic group, which is a group that can be protonated or deprotonated, depending on the pH.

Unless specified otherwise, the term “patient” refers to any mammalian animal, including but not limited to, humans.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made. Lists of additional suitable salts may be found, e.g., in Remington's Pharmaceutical Sciences, 17th ed., Mack Publishing Company, Easton, Pa., p. 1418 (1985).

In some embodiments, the RNA polymerase inhibitor can be a compound of Formula (1):

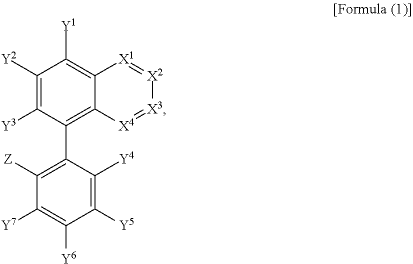

or a pharmaceutically acceptable salt thereof, wherein:

X¹ is selected from CR¹ or N;

X² is selected from CR² or N;

X³ is selected from CR³ or N;

X⁴ is selected from CR⁴ or N;

Z is selected from:

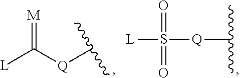

—(CH₂)ₙL, C₁₋₁₀heterocycle, or C₁₋₁₀heteroaryl;

n is from 0-10;

L is selected from R⁵; OR⁵, or NR⁵R⁶;

Y¹ is selected from —F, —Cl, —Br, —I, —NO₂, —CN, -Q¹R^{a}, —N(R^{a})₂, -Q¹C(M)Q¹R^{a}, -Q¹C(O)N(R^{a})₂, -Q¹SO₂Q¹R^{a}, or -Q¹SO₂)N(R^{a})₂,

Y² is selected from —F, —Cl, —Br, —I, —NO₂, —CN, -Q¹R^{b}, —N(R^{b})₂, -Q¹C(M)Q¹R^{b}, -Q¹C(O)N(R^{b})₂, -Q¹SO₂Q¹R^{b}, or -Q¹SO₂N(R^{b})₂,

Y³ is selected from —F, —Cl, —Br, —I, —NO₂, —CN, -Q¹R^{c}, N(R^{c})₂, -Q¹C(M)Q¹R^{c}, -Q¹C(M)N(R^{c})₂, -Q¹SO₂Q¹R^{c}, or -Q¹SO₂N(R^{c})₂,

Y⁴ is selected from —F, —Cl, —Br, —I, —NO₂, —CN, -Q¹R^{d}, —N(R^{d})₂, -Q¹C(M)Q¹R^{d}, -Q¹C(M)N(R^{d})₂, -Q¹SO₂Q¹R^{d}, or -Q¹SO₂N(R^{d})₂,

Y⁵ is selected from —F, —Cl, —Br, —I, —NO₂, —CN, -Q¹R^{e}, —N(R^{e})₂, -Q¹C(M)Q¹R^{e}, -Q¹C(M)N(R^{e})₂, -Q¹SO₂Q¹R^{e}, or -Q¹SO₂N(R^{e})₂,

Y⁶ is selected from —F, —Cl, —Br, —I, —NO₂, —CN, -Q¹R^{f}, —N(R^{f})₂, -Q¹C(M)Q¹R^{f}, -Q¹C(M)N(R^{f})₂, -Q¹SO₂Q¹R^{f}, or -Q¹SO₂N(R^{f})₂,

Y⁷ is selected from —F, —Cl, —Br, —I, —NO₂, —CN, -Q¹R^{g}, —N(R^{g})₂, -Q¹C(M)Q¹R^{g}, -Q¹C(M)N(R^{g})₂, -Q¹SO₂Q¹R^{g}, or -Q¹SO₂N(R^{g})₂,

M is in each case independently selected from O, NH, S, NOH, or CH;

Q is in each case independently selected from null, O, NH, or S;

Q¹ is in each case independently selected from null, O, NH, or S;

R¹, R², R³, and R⁴, are independently selected from R^{p}, OR^{p}, N(R^{p})₂, CN, NO₂, COR^{p}, C(O)OR^{p}, Fl, Cl, Br, or I, wherein R^{p} is in each case independently selected from H, C₁₋₁₀alkyl, C₁₋₁₀haloalkyl, aryl, C₁₋₁₀heterocycle, or C₁₋₁₀heteroaryl;

R⁵, R⁶, R^{a}, R^{b}, R^{c}, R^{d}, R^{e}, R^{f}, and R^{g} are in each case independently selected from H, C₁₋₁₀alkyl, C₁₋₁₀haloalkyl, aryl, C₁₋₁₀heterocycle, or C₁₋₁₀heteroaryl.

wherein any two or more of L, R¹, R², R³, R⁴, R⁵, R⁶, Y¹, Y², Y³, Y⁴, Y⁵, Y⁶, Y⁷, R^{a}, R^{b}, R^{c}, R^{d}, R^{e}, R^{f}, and R^{g} can together form a ring.

In certain embodiments, Y⁴ is H, while in other embodiments, R⁴ is selected from OH, F, Cl, Br, I, NO₂, CN, CO₂H, or C₁₋₈ alkyl, such as methyl, ethyl, propyl, isopropyl, methoxy, ethoxy, or isopropoxy. In some embodiments, Y⁴ and Y⁵ together form a ring, for instance an aryl, heteroaryl, cycloalkyl or heterocyclyl ring. Exemplary systems include:

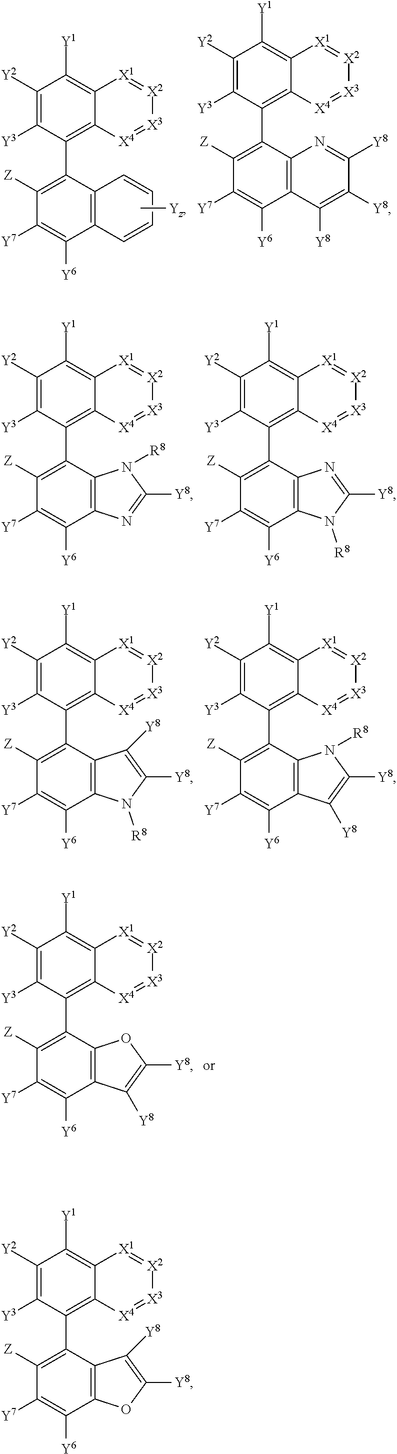

wherein R⁸ is selected from H or C₁₋₃alkyl, and Y⁸ is in each case independently selected from R^{p}, OR^{p}, N(R^{p})₂, CN, NO₂, COR^{p}, C(O)OR^{p}, Fl, Cl, Br, or I, wherein R^{p} is in each case independently selected from H, C₁₋₁₀alkyl, C₁₋₁₀haloalkyl, aryl, C₁₋₁₀heterocycle, or C₁₋₁₀heteroaryl. Structures depicted in which the Y⁸ group is not directly connected to a specific atom should be understood to include compounds in which Y⁸ is connected to any, or multiple possible atoms. Additional heteroaryl groups include oxazoles, thiazoles, triazoles, diazines, dihydrofurans, dihydropyrans and the like.

Exemplary Z heterocycles and heteroaryls include tetrazoles, triazoles, oxazoles, imidazoles, pyrrolidines, piperidines, furans, pyrans, and the like. In some embodiments, the RNA polymerase inhibitor can have the formula:

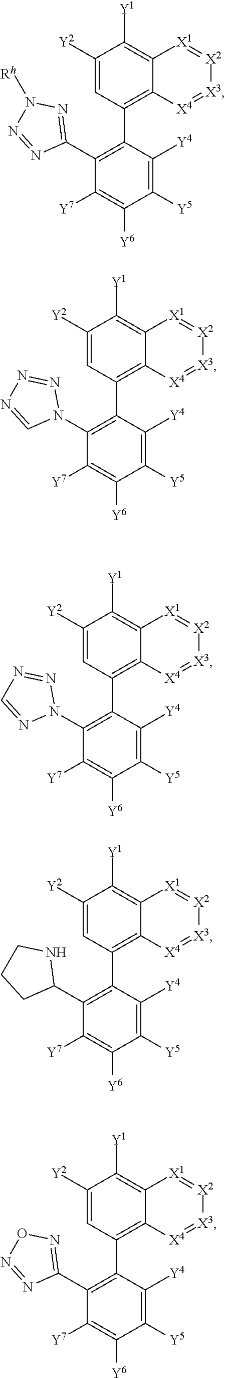

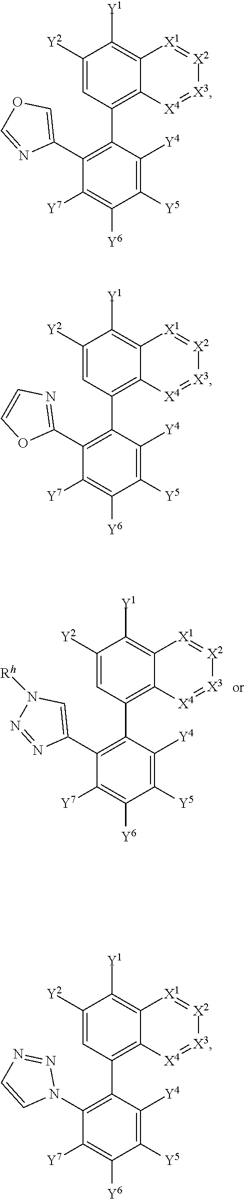

wherein R^{h} can be hydrogen or C₁₋₃alkyl. Although each of the carbon atoms in the depicted heterocycle and heteroaryl groups above are unsubstituted, the heterocycle and heteroaryl groups can be further substituted.

Exemplary C₁₋₁₀haloalkyl groups include CX₃, CX₂H, CXH₂, CH₂CX₃, CH₂CX₂H, CH₂CXH₂, CX₂CX₃, etc, wherein X is F, Cl, Br, I, or combinations thereof. In some embodiments, X is F.

In certain instances, the compound of Formula (1) can be a benzopyridine derivative, i.e., only one of X¹, X², X³, and X⁴ is N, while in other embodiments the compound of Formula (1) is a benzopyrimdine, i.e., two of X¹, X², X³, and X⁴ are N. In yet further embodiment, the compound of Formula (1) can be a benzotriazine, i.e., three of X¹, X², X³, and X⁴ are N, and in other embodiments, the compound of Formula (1) is a benzotetrazine.

In some embodiments, the compound of Formula (1) is a benzopyridine wherein X¹ is N, X² is CR², X³ is CR³, and X⁴ is CR⁴. In other embodiments, the compound of Formula (1) is a benzopyridine, wherein X² is N, X¹ is CR¹, X³ is CR³, and X⁴ is CR⁴. In other instances, X³ is N, X¹ is CR¹, X² is CR², and X⁴ is CR⁴; or X⁴ is N, X¹ is CR¹, X² is CR², and X³ is CR³.

In certain instances, Z can be a group having the formula:

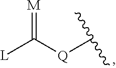

wherein M is O and Q is null. In such cases, L can be NR⁵R⁶ wherein R⁵ and R⁶ are each selected from hydrogen or C₁₋₃alkyl. In certain embodiments, R⁵ can be H, and R⁶ can be selected from hydrogen or C₁₋₃alkyl, preferably methyl or H.

In some embodiments, Y⁷ can be selected from F, Cl, Br, NO₂, CN, or —C(M)Q¹R^{c}, wherein M is O, Q¹ is null or O, and R^{c} is H, C₁₋₁₀alkyl, aryl, C₁₋₁₀heterocycle, or C₁₋₁₀heteroaryl.

Examples of Formula I compounds are shown below:

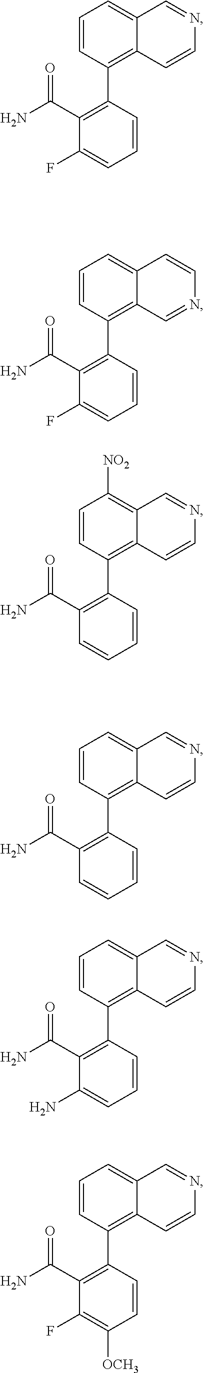

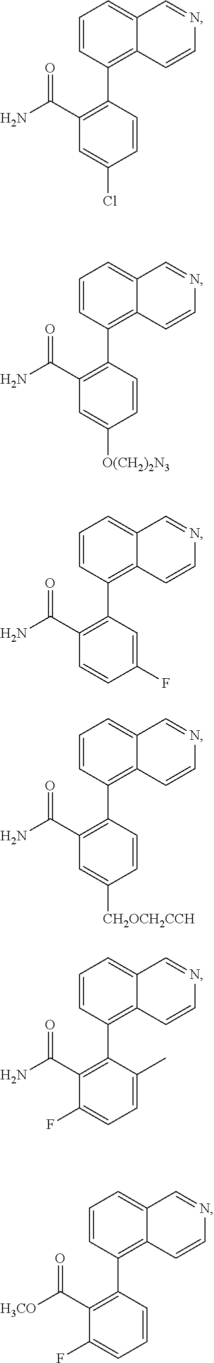

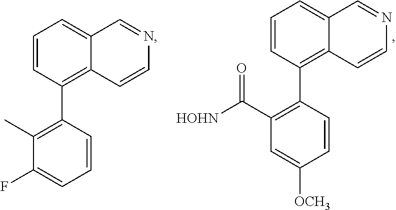

and pharmaceutically acceptable salts

In certain embodiments, the RNA polymerase inhibitor can be a compound of Formula (2):

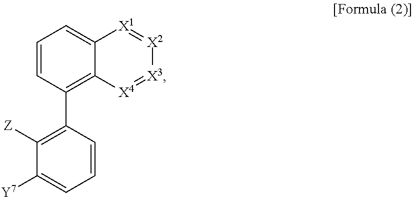

or a pharmaceutically acceptable salt thereof, wherein Z, Y⁷, X¹, X², X³, and X⁴ have the meanings given above. In certain embodiments, the compound of Formula (2) can be further characterized by X⁴=CR⁴, wherein R⁴ is H, methyl, ethyl, propyl, isopropyl, methoxy, ethoxy, isopropoxy, F, Cl, Br, I, NO₂, or CN.

In certain embodiments, the RNA polymerase inhibitor can be a compound of Formula (3):

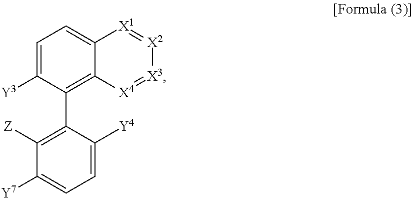

or a pharmaceutically acceptable salt thereof, wherein Z, X³, and X⁴ have the meanings given above. In certain embodiments, the compound of Formula (2) can be further characterized by X⁴=CR⁴, wherein R⁴ is H, methyl, ethyl, propyl, isopropyl, methoxy, ethoxy, isopropoxy, F, Cl, Br, I, NO₂, or CN. In some embodiments, Y⁴ is selected from H, methyl, ethyl, propyl, isopropyl, methoxy, ethoxy, or isopropoxy.

In some embodiments, the RNA polymerase inhibitor can be a compound of Formula (4a) or (4b):

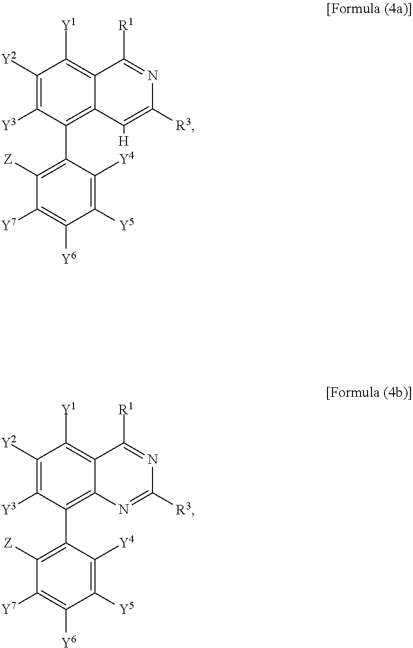

wherein R¹, R³, Y¹, Y², Y³, Y⁴, Y⁵, Y⁶, Y⁷ and Z are as defined above, and in some cases each of each of Y¹, Y², Y³, Y⁴, and Y⁶ are hydrogen. In certain embodiments, one or both of R¹ and R³ can be OH, NH₂ or H, for instance R³ can be OH or NH₂, and R¹ is H, R¹ can be OH or NH₂ and R³ is H, R¹ and R³ are each H, or R¹ and R³ are each OH or NH₂.

In certain embodiments, the compounds of Formula (4a) and (4b) can be characterized by each of groups Y¹, Y², Y³, Y⁴, Y⁵, and Y⁶ being hydrogen. In certain instances of Formula (4a) and (4b), Z can be a C₁₋₁₀heteroaryl group, or a group having the formula:

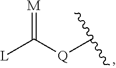

wherein M, L, and Q are as defined above. In certain embodiments of the compounds of Formula (4a) and (4b), Q can be O or null, preferably null, M can be O, and L can be OH or NH₂. Alternatively, L can be OR⁵ or NR⁵R⁶, wherein R⁶ is hydrogen, and R⁵ is C₁₋₃alkyl group, or R⁵ together with Y⁷ or Y³ together form a ring. In such cases, L can be NR⁵R⁶ wherein R⁵ and R⁶ are each selected from hydrogen or C₁₋₃alkyl. In other cases, Y⁷ is selected from H, F, Cl, Br, I, NO₂, or CN.

In some embodiments, the compound of Formula (4a) can be characterized in which Y², Y³, Y⁴, Y⁵, and Y⁶, are each hydrogen, and Y⁷ is selected from —H —F, —Cl, —Br, —I, or —N(R^{g})₂, wherein R^{g} is as defined above. In particular embodiments, R^{g} is in each case independently selected from H, or C₁₋₁₀ alkyl, and in further embodiments R^{g} is in both instances H. In other embodiments, Y², Y³, Y⁴, Y⁵, Y⁷ are each hydrogen, and Y⁶ is selected —F, —Cl, —Br, —I, or Q¹R^{f}, wherein Q¹ and R^{f} are as defined above. In certain embodiments, Q¹ is null or O, and R^{f} is H or C₁₋₁₀alkyl. In other embodiments, Y², Y³, Y⁵, Y⁶, are each hydrogen, and Y⁷ is selected from —F, —Cl, —Br, —I, or —N(R^{g})₂, and Y⁴ is Q¹R^{d}, wherein each of Q¹, R^{d}, and R^{g} is as defined above. In certain embodiments, Q¹ is null or O, R^{d} is H or C₁₋₁₀alkyl, and R^{g} is in each case independently selected from H, or C₁₋₁₀alkyl, and in further embodiments R^{g} is in both instances H.

In certain embodiments for compounds of Formula (4a), Z is selected from:

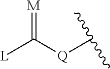

wherein L, M, and Q are as defined above. In particular embodiments, L is OR⁵ or NR⁵R⁶, M is O, NH, or NOH, and R⁵ and R⁶ are in each case independently selected from hydrogen or C₁₋₁₀ alkyl. In certain embodiments, Q can be null.

In other embodiments for compounds of Formula (4a), Z is selected from:

—(CH₂)ₙL,

wherein L and n are as defined above. In certain embodiments, L can be R⁵ which can be H.

In some embodiments for the compound of Formula (4a), Y¹ can be Q¹R^{a} or NO₂, wherein Q¹ and R^{a} are as defined above. In some embodiments where Y¹ is Q¹R^{a}, Q¹ can be null or O, and R^{a} can be H or C₁₋₁₀alkyl.

In other embodiments, the RNA polymerase inhibitor can be a compound of Formula (4c) or (4d):

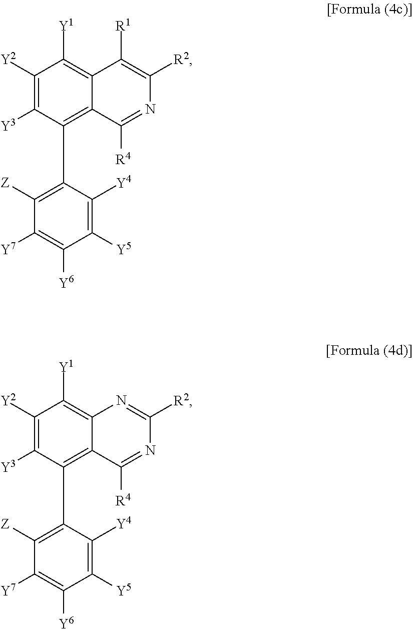

wherein R¹, R², and R⁴ are as defined above, and each of Y¹, Y², Y³, Y⁴, Y⁶, Y⁷, and Z are as defined above, and in some cases each of each of Y¹, Y², Y³, Y⁴, and Y⁶ are hydrogen. In certain embodiments, one or both of R² and R⁴ can be OH, NH₂ or H, for instance R² can be OH or NH₂ and R⁴ is H, R⁴ can be OH or NH₂ and R² is H, R⁴ and R² are each H, or R⁴ and R² are each OH or NH₂. In certain instances of Formula (4c) and (4d), Z can be a C₁₋₁₀heteroaryl group, or a group having the formula:

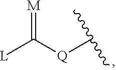

wherein M, L, and Q are as defined above. In certain embodiments of the compounds of Formula (4c) and (4d), Q can be O or null, preferably null, M can be O, and L can be OH or NH₂. Alternatively, L can be OR⁵ or NR⁵R⁶, wherein R⁶ is hydrogen, and R⁵ is C₁₋₃alkyl group, or R⁵ together with Y³ together form a ring. In such cases, L can be NR⁵R⁶, wherein R⁵ and R⁶ are each selected from hydrogen or C₁₋₃alkyl. In some embodiments of the compounds of Formula (4c) and (4d), Y⁵ is selected from H, F, Cl, Br, I, NO₂, or CN.

In other embodiments, the RNA polymerase inhibitor can be a compound of Formula (4e), (4f) or (4g):

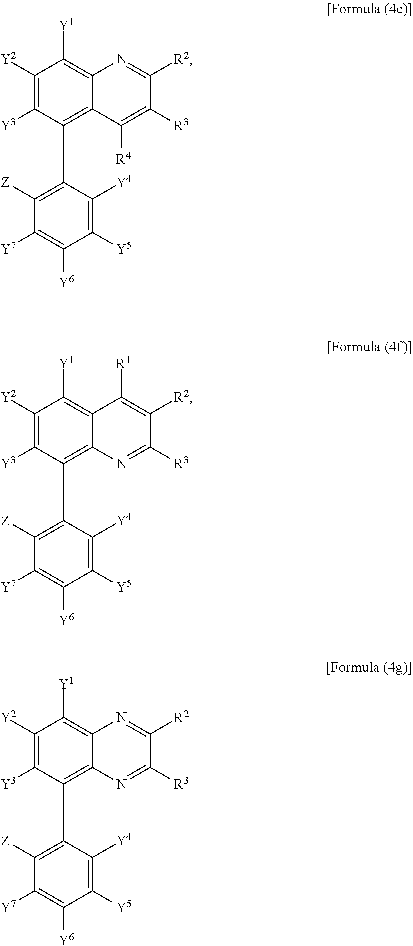

wherein R¹, R², R³, and R⁴ are as defined above, and each of Y¹, Y², Y³, Y⁴, Y⁶, Y⁷, and Z are as defined above, and in some cases each of each of Y¹, Y², Y³, Y⁴, and Y⁶ are hydrogen. In certain embodiments, one or both of R² and R³ can be OH, NH₂ or H, for instance R² can be OH or NH₂ and R³ is H, R³ can be OH or NH₂ and R² is H, R³ and R² are each H, or R³ and R² are each OH or NH₂. In certain embodiments of the compound of Formula (4e), R⁴ can NH₂, while in certain embodiments of Formula (4f), R¹ can be NH₂. In certain instances of Formula (4e), (4f), and (4g), Z can be a C₁₋₁₀heteroaryl group, or a group having the formula:

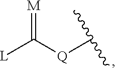

wherein M, L, and Q are as defined above. In certain embodiments of the compounds of Formula (4e), (4f), and (4g), Q can be O or null, preferably null, M can be O, and L can be OH or NH₂. Alternatively, L can be OR⁵ or NR⁵R⁶, wherein R⁶ is hydrogen, and R⁵ is C₁₋₃alkyl group, or R⁵ together with Y³ together form a ring. In such cases, L can be NR⁵R⁶ wherein R⁵ and R⁶ are each selected from hydrogen or C₁₋₃alkyl. In some embodiments of the compounds of Formula (4e), (4f), and (4g), Y⁵ is selected from H, F, Cl, Br, I, NO₂, or CN.

In certain embodiments, Y¹ and Y⁴ can together form a ring, for instance Y¹ and Y² together from a group

In some embodiments, Z and Y³ can together form a ring:

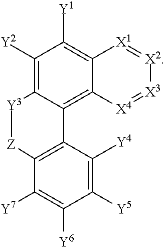

Exemplary ring systems include those having the formula:

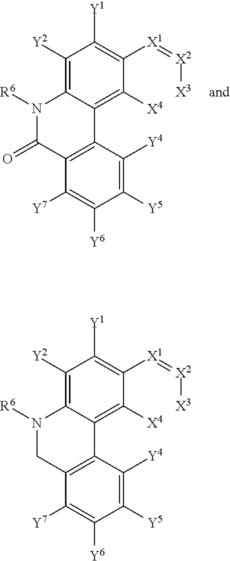

wherein Y¹, Y², Y⁴, Y⁵, Y⁶, R⁶, Y⁷, X¹, X², X³, and X⁴ have the meanings given above. In other embodiments, Z and Y⁷ can together form a ring:

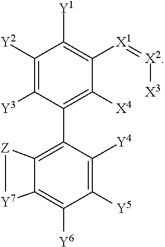

Exemplary ring systems include

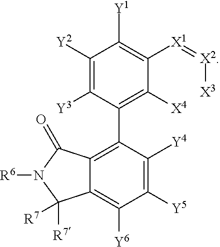

The compounds disclosed herein may be formulated in a wide variety of pharmaceutical compositions for administration to a patient. Such compositions include, but are not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

The compounds disclosed herein may be administered by a number of different routes. For instance, the compounds may be administered orally, topically, transdermally, intravenously, subcutaneously, by inhalation, or by intracerebroventricular delivery.

In some embodiments, the compounds disclosed herein may be formulated as nanoparticles. The nanoparticles may have an average particle size from 1-1,000 nm, preferably 10-500 nm, and even more preferably from 10-200 nm.

The compounds disclosed herein possess RNA polymerase inhibitory activity, and as such are useful for the treatment of viral infections caused by susceptible viruses. In some instances, the compounds can be used to treat one or more of the Paramyxoviridae family of negative-sense RNA viruses, for instance, human parainfluenza viruses (HPIV, types 1-4) or measles virus (MeV). In other embodiments, the compounds disclosed herein may be used to treat other viral infections, including Respiratory syncytial virus (RSV), influenza A virus including subtype H1N1, influenza B virus, influenza C virus, rotavirus A, rotavirus B, rotavirus C, rotavirus D, rotavirus E, SARS coronavirus, human adenovirus types (HAdV-1 to 55), human papillomavirus (HPV) Types 16, 18, 31, 33, 35, 39, 45, 51, 52, 56, 58, 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, mumps virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, Kaposi's sarcoma-associated herpesvirus, hepatitis A (HAV), hepatitis B (HBV), hepatitis C (HCV), hepatitis D (HDV), hepatitis E (HEV), human immunodeficiency virus (HIV), The Human T-lymphotropic virus Type I (HTLV-1), Friend spleen focus-forming virus (SFFV) or Xenotropic MuLV-Related Virus (XMRV).

The compounds disclosed herein may be administered in combination with one or more additional anti-viral agents. In certain embodiments, methods disclosed herein are contemplated to be administered in combination with other the antiviral agent(s) such as abacavir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate, tipranavir, trifluridine, trizivir, tromantadine, truvada, valaciclovir, valganciclovir, vicriviroc, vidarabine, viramidine, zalcitabine, zanamivir, or zidovudine, and combinations thereof.

### EXAMPLES

The following examples are for the purpose of illustration of the invention only and are not intended to limit the scope of the present invention in any manner whatsoever.

Driven by the rationale that a sizeable adult patient population and viable treatment window will be paramount for advance to clinical testing, HPIVs represent a promising primary target for an anti-paramyxovirus drug screen. In addition to children, HPIVs pose a major threat to immune-compromised adults such as hematopoietic stem-cell transplant patients, among whom case-fatality rates can reach a staggering 75%. HPIV disease progression in some adult at-risk groups appears to be relatively slow, reflected by a median 3 days for progression to severe lower respiratory tract infection after appearance of initial upper respiratory symptoms. In this study, we therefore selected HPIV3, the predominant etiological agent of HPIV disease with an estimated 3 million medically-attended cases in the US annually, as the screening agent for a high-throughput antiviral drug discovery campaign. This screen identified GHP-88309, an orally efficacious broad-spectrum inhibitor of the paramyxovirus polymerase.

### Example 1: Synthesis of GHP-88309

### Chemicals

All materials were obtained from commercial suppliers and used without further purification, unless otherwise noted. Dry organic solvents, packaged under nitrogen in septum sealed bottles, were purchased from EMD Millipore and Sigma-Aldrich Co. Reactions were monitored using EMD silica gel 60 F254 TLC plates or using an Agilent 1200 series LCMS system with a diode array detector and an Agilent 6120 quadrupole MS detector. Compound purification was accomplished by liquid chromatography on a Teledyne Isco CombiFlash RF+ flash chromatography system. NMR spectra were recorded on an Agilent NMR spectrometer (400 MHz) at room temperature. Chemical shifts are reported in ppm relative to residual DMSO-d6 signal. The residual shifts were taken as internal references and reported in parts per million (ppm). The screening collection was assembled from commercial libraries (ChemBridge and ChemDiv), both curated against chemical structures with undesirable reactivity, and a proprietary compound collection derived from previous medicinal chemistry optimization campaigns. All compounds were dissolved in DMSO to a concentration of 10 μM and stored at −80° C. To generate a screening set, all compounds were inventoried in MScreen and reformatted into barcoded 384-well format daughter plates using a Nimbus96 liquid handler (Hamilton Robotics). Thirty-two wells on each 384-well plate were reserved for positive and negative (vehicle) controls, arranged in a checkerboard pattern in the two lateral columns of either side.

### Chemical Synthesis

Synthesis of 2-fluoro-6-(5-isoquinolyl)benzonitrile

2-bromo-6-fluoro-benzonitrile (5.0 gm, 25 mmol), 5-isoquinolylboronic acid (6.0 gm, 35 mmol) and Na₂CO₃ (10.6 gm, 100 mmol) were placed in a 250 ml sealed flask and treated with (2:1)1,4-dioxane:water (90 ml). The mixture was purged with argon for 5 min. Pd(PPh₃)₄ (2.25 gm, 1.94 mmol) was added and purged for another 5 minutes. The reaction mixture was sealed and stirred at 100° C. for 3-5 hours. After completion, the reaction mixture was cooled to room temperature, diluted with excess dichloromethane and extracted with water. The aqueous layer was extracted with dichloromethane, the combined organic layers dried over anhydrous Na₂SO₄, filtered, and concentrated under reduced pressure. The crude product was purified by flash column chromatography using dichloromethane and methanol as eluent. Pure product was obtained as colorless solid, yield 77% (4.8 gm).

¹H NMR 400 MHz, DMSO-d6, δ 9.44 (1H, s), 8.51 (1H, d, J=8 Hz), 8.30 (1H, d, J=8 Hz), 7.96-7.66 (3H, m), 7.68 (1H, t, 8 Hz), 7.50 (1H, d, J=8 Hz), 7.42 (1H, d, J=4 Hz); ¹⁹F NMR 376 MHz, DMSO-d6 δ −107.10 to −107.14, (1F, m); MS (ES-API) [M+1]+: 249.0.

Synthesis of 2-fluoro-6-(5-isoquinolyl)benzamide (GHP-88309)

2-fluoro-6-(5-isoquinolyl)benzonitrile (4 gm, 16.1 mmol) was placed in a 100 ml round bottom flask and treated with acetic acid (12 ml, 209 mmol) and conc. H₂SO₄ (6 ml, 112 mmol). The reaction mixture was stirred at 120° C. for 2-3 hours. After completion, the reaction mixture was cooled to room temperature and poured on crushed ice and carefully neutralized with aq. NaOH. Aqueous mixture was extracted thrice with ethyl acetate, organic layers were combined, dried over anhydrous Na₂SO₄, filtered and concentrated under reduced pressure. The crude product was purified by flash column chromatography using dichloromethane and methanol as eluent. Pure product was obtained as colorless solid, yield 46% (2 gm).

¹H NMR 400 MHz, DMSO-d6, δ 9.34 (1H, s), 8.43 (1H, d, J=4 Hz), 8.14 (1H, ddd, J=8 Hz, 2 Hz, 0.8 Hz), 7.87 (1H, bs), 7.70 (2H, m), 7.54 (1H, m), 7.39 (³H, m), 7.16 (1H, dd, J=8 Hz, 0.8 Hz); ¹⁹F NMR 376 MHz, DMSO-d6 δ −116.27 to −116.31, (1F, m); ¹³C NMR 100 MHz, DMSO-d6, δ 165.81, 158.75 (d, J=250 Hz), 152.93, 143.54, 138.39, 136.05, 134.18, 131.66, 130.40 (d, J=9 Hz), 128.68, 128.10, 127.81, 127.61, 127.10, 119.08, 115.55 (d, J=22 Hz); MS (ES-API) [M+1]+: 267.0.

Synthesis of Chemical Analogs

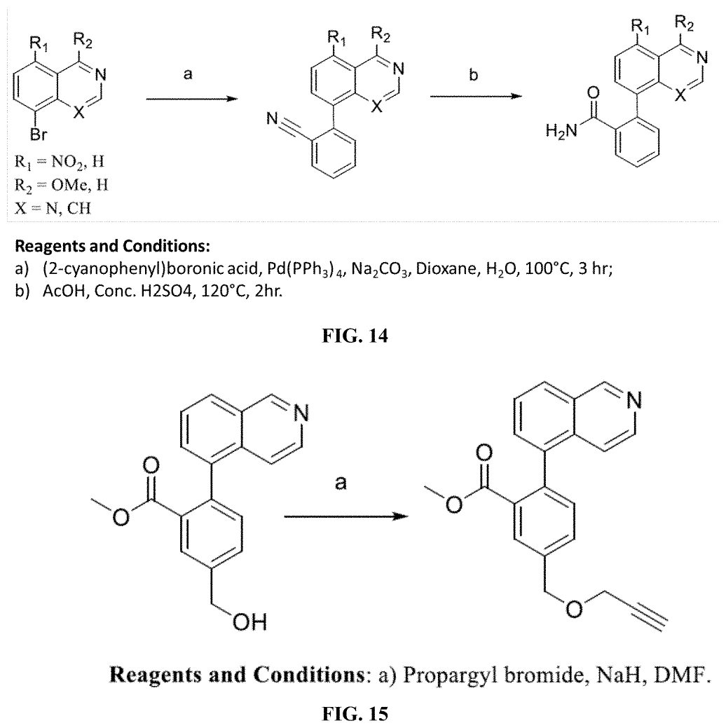

Analogs were generated using the same procedure described for the synthesis of GHP-88309, except GHP-88309-003, GHP-88309-004, GHP-88309-009, GHP-88309-010 and GHP-88309-015. Synthesis of GHP-88309-003 and GHP-88309-004 was achieved by the reaction of 5-bromo-8-nitroquinolone and 8-bromo-4-methoxyquinazoline respectively with (2-cyanophenyl) boronic acid as shown in FIG. 14.

In the case of GHP-88309-009 containing acid labile alkyl ether, nitrile to amide conversion was achieved through alternate basic condition¹ as shown in FIG. 14. In GHP-88309-010, propargyl group was introduced after boronic acid coupling reaction to avoid potential interference from the alkyne function as shown in FIG. 14.

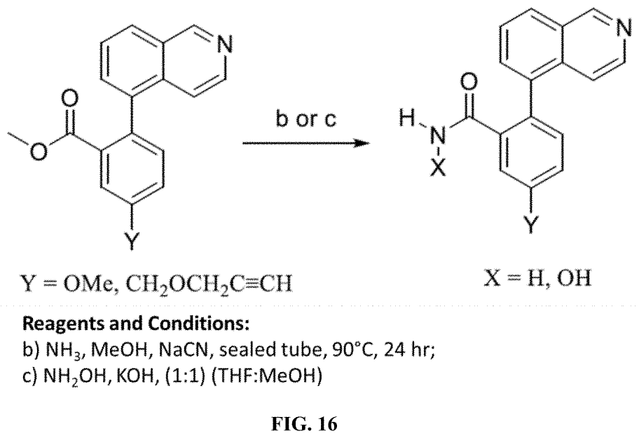

Analogs GHP-88309-010 and GHP-88309-015 were obtained by the conversion of esters to the corresponding amides using condition (b) and (c) respectively as shown in FIG. 16.

Analytical Data

GHP-88309-001: ¹H NMR (400 MHz, Methanol-d₄) δ 8.82 (d, J=4 Hz, 1H), 8.09-8.04 (m, 2H), 7.77 (dd, J=8 Hz, 4 Hz, 1H), 7.58-7.51 (m, 2H), 7.44 (dd, J=4 Hz, 1H), 7.31-7.26 (m, 1H), 7.18 (dd, J=8 Hz, 1 Hz, 1H); ¹⁹F NMR (376 MHz, Methanol-d₄) δ −115.81 (dd, J=5.6 Hz, J=9.4 Hz); MS (ES-API) [M+1]⁺: 267.1.

GHP-88309-002: ¹H NMR (400 MHz, Methanol-d₄) δ 8.95 (s, 1H), 8.42 (d, J=5.8 Hz, 1H), 7.95 (d, J=9.0 Hz, 1H), 7.84 (d, J=5.8 Hz, 1H), 7.81-7.77 (m, 1H), 7.63-7.54 (m, 1H), 7.59-7.52 (m, 1H), 7.35-7.30 (m, 1H), 7.23 (d, J=7.6 Hz, 1H); ¹⁹F NMR (376 MHz, Methanol-d₄) δ −116.34 (dd, J=9.3, 5.7 Hz); MS (ES-API) [M+1]⁺: 267.1.

GHP-88309-003: ¹H NMR (400 MHz, Methanol-d₄) δ 9.90 (s, 1H), 8.54 (d, J=6.0 Hz, 1H), 8.47 (d, J=7.9 Hz, 1H), 7.84-7.76 (m, 2H), 7.71-7.62 (m, 2H), 7.60 (dd, J=6.0 Hz, 1.0 Hz, 1H), 7.46-7.42 (m, 1H). MS (ES-API) [M+1]⁺: 293.9.

GHP-88309-004: ¹H NMR (400 MHz, Methanol-d₄) δ 8.63 (s, 1H), 8.23 (dd, J=8.3 Hz, 1.5 Hz, 1H), 7.86 (dd, J=7.3 Hz, 1.5 Hz, 1H), 7.73-7.65 (m, 2H), 7.59 (td, J=7.5, 1.5 Hz, 1H), 7.52 (td, J=7.5, 1.4 Hz, 1H), 7.43 (dd, J=7.5, 1.4 Hz, 1H), 4.21 (s, 3H). MS (ES-API) [M+1]⁺: 280.0.

GHP-88309-005: ¹H NMR (400 MHz, Chloroform-d) δ 9.43 (broad s, 1H), 8.40 (broad s, 1H), 8.13 (d, J=8 Hz, 1H), 7.88-7.80 (m, 3H), 7.70 (broad s, 1H), 7.65-7.57 (m, 2H). 7.37 (dd, J=8 Hz, 1 Hz, 1H), 5.66 (broad s, 2H); MS (ES-API) [M+1]⁺: 249.1.

GHP-88309-006: ¹H NMR (400 MHz, Methanol-d₄) δ 9.26 (s, 1H), 8.35 (d, J=4 Hz, 1H), 8.05 (d, J=8 Hz, 1H), 7.76-7.68 (m, 3H), 7.25 (dd, J=8 Hz, 1H), 6.84 (dd, J=8 Hz, 1 Hz, 1H), 6.63 (dd, J=8 Hz, 1 Hz, 1H). MS (ES-API) [M+1]⁺: 264.1.

GHP-88309-007: ¹H NMR (400 MHz, DMSO-d₆) δ 9.38 (s, 1H), 8.44 (d, J=5.9 Hz, 1H), 8.16 (d, J=7.8 Hz, 1H), 7.89 (s, 1H), 7.77-7.66 (m, 2H), 7.48 (d, J=5.9 Hz, 1H), 7.39 (s, 1H), 7.32 (pseudo t, J=8.6 Hz, 1H), 7.11 (d, J=8.4 Hz, 1H), 3.94 (s, 3H). ¹⁹F NMR (376 MHz, DMSO-d₆) δ −138.50 (d, J=8.8 Hz). MS (ES-API) [M+1]⁺: 297.1.

GHP-88309-008: ¹H NMR (400 MHz, Methanol-d₄) δ 9.30 (s, 1H), 8.25 (s, 1H), 8.09 (dd, J=7 Hz, 2 Hz, 1H), 7.73-7.68 (m, 2H), 7.60-7.56 (m, 2H), 7.42-7.40 (m, 1H), 7.22 (d, J=2 Hz, 1H). MS (ES-API) [M+1]⁺: 283.1.

GHP-88309-009: ¹H NMR (400 MHz, Methanol-d₄) δ 9.25 (s, 1H), 8.35 (d, J=6.1 Hz, 1H), 8.10 (dd, J=6.4 Hz, 3.1 Hz, 1H), 7.74-7.69 (m, 2H), 7.55 (d, J=6.1 Hz, 1H), 7.34 (d, J=8.4 Hz, 1H), 7.30 (d, J=2.7 Hz, 1H), 7.22 (dd, J=8.4 Hz, 2.7 Hz, 1H), 4.31 (t, J=4.8 Hz, 2H), 3.6 (t, J=4.8 Hz, 2H); MS (ES-API) [M+1]⁺: 333.9.

GHP-88309-010: ¹H NMR (400 MHz, Methanol-d₄) δ 9.26 (s, 1H), 8.34 (d, J=6.0 Hz, 1H), 8.12 (dd, J=6.8 Hz, 1H), 7.77-7.68 (m, 3H), 7.64-7.58 (m, 1H), 7.53 (d, J=6.0 Hz, 1H), 7.40 (d, J=7.8 Hz, 1H), 4.75 (s, 2H), 4.30 (d, J=2.4 Hz, 2H), 2.96 (t, J=2.4 Hz, 1H); MS (ES-API) [M+1]⁺: 317.1.

GHP-88309-011: 1H NMR (400 MHz, Chloroform-d) δ 9.34 (broad s, 1H), 8.49 (broad s, 1H), 8.09-8.05 (m, 1H), 7.97 (dd, J=8 Hz, 4 Hz, 1H), 7.73-7.69 (m, 2H), 7.43 (d, J=8 Hz, 1H), 7.29-7.24 (m, 1H). 7.07 (dd, J=8 Hz, 1 Hz, 1H); ¹⁹F NMR (376 MHz, Chloroform-d) δ −108.31 to −108.36, (m). MS (ES-API) [M+1]⁺: 267.1.

GHP-88309-012: ¹H NMR (400 MHz, Methanol-d₄) δ 9.29 (s, 1H), 8.37 (s, 1H), 8.17 (d, J=8.1 Hz, 1H), 7.78-7.74 (m, 1H), 7.69 (d, J=6.8 Hz, 1H), 7.47-7.44 (m, 1H), 7.33 (d, J=4.5 Hz, 1H), 7.27-7.23 (m, 1H), 1.87 (s, 3H); ¹⁹F NMR (376 MHz, Methanol-d₄) δ −122.16 to −122.20, (m). MS (ES-API) [M+1]⁺: 281.1.

GHP-88309-013: ¹H NMR (400 MHz, Chloroform-d) δ 9.39 (s, 1H), 8.49 (s, 1H), 8.09 (d, J=8.0 Hz, 1H), 7.75-7.63 (m, 2H), 7.60-7.50 (m, 2H), 7.29 (dd, J=7.6 Hz, 0.8 Hz, 1H), 7.19 (dd, J=7.6 Hz, 0.8 Hz, 1H), 3.42 (s, 3H). ¹⁹F NMR (376 MHz, Chloroform-d) δ −113.33 (dd, J=9.4, 5.2 Hz); MS (ES-API) [M+1]⁺: 282.1.

GHP-88309-014: ¹H NMR (400 MHz, Chloroform-d) δ 9.32 (s, 1H), 8.46 (d, J=5.6 Hz, 1H), 8.01 (d, J=8 Hz, 1H), 7.67 (dd, J=8 Hz, 7.2 Hz, 1H), 7.56 (dd, J=7.2 Hz, 1.2 Hz, 1H), 7.32-7.21 (m, 2H), 7.14 (pseudo t, J=8.4 Hz, 1H), 7.02 (d, J=7.6 Hz, 1H), 1.93 (s, 3H); ¹⁹F NMR (376 MHz, Chloroform-d) δ −115.68 to −115.77 (m). MS (ES-API) [M+1]⁺: 238.1.

GHP-88309-015: ¹H NMR (400 MHz, Methanol-d₄) δ 9.24 (d, J=0.8 Hz, 1H), 8.34 (d, J=6 Hz, 1H), 8.10 (td, J=4.4 Hz, 0.8 Hz, 1H), 7.71 (dd, J=4.8 Hz, 0.8 Hz, 2H), 7.52 (dt, J=6 Hz, 0.8 Hz, 1H), 7.33 (dd, J=8.4 Hz, 0.8 Hz, 1H), 7.25-7.13 (m, 2H), 3.92 (s, 3H). MS (ES-API) [M+1]⁺: 295.0.

GHP-88309-016: ¹H NMR (400 MHz, Methanol-d₄) δ 9.26 (s, 1H), 8.35 (d, J=6.0 Hz, 1H), 8.13 (m, 1H), 7.73 (d, J=4.9 Hz, 2H), 7.52 (dd, J=6 Hz, 1 Hz, 1H), 7.46-7.41 (m, 1H), 7.40-7.37 (m, 1H), 7.35-7.32 (m, 1H); MS (ES-API) [M+1]⁺: 290.0.

Cells

Human carcinoma (HEp-2, ATCC CCL-23), human embryonic kidney (293T, ATCC CRL-3216), human bronchial epithelial (BEAS-2B, ATCC CCL-9609), human Madin Darby canine kidney (MDCK, ATCC CCL-34), African green monkey kidney epithelial cells (CCK-81; ATCC) stably expressing human signaling lymphocytic activation molecule (Vero-hSLAM) or canine signaling lymphocytic activation molecule (Vero-cSLAM) were maintained at 37° C. and 5% CO₂ in Dulbecco's modified Eagle's medium (DMEM) supplemented with 7.5% fetal bovine serum. HEp-2 cells are indicated in the ICLAC database of commonly misidentified cell lines, use of these cells is necessary since they are highly permissive for respiratory syncytial virus. Normal primary Human Bronchial Tracheal Epithelial Cells (HBTECs; Lifeline Cell Technology) were propagated and maintained in BronchiaLife Cell Culture Medium at 37° C. and 5% CO₂. HBTECs were analyzed for microbial contamination by LifeLine Cell Technology. Insect cells (SF9) were propagated in suspension using Sf-900 II SFM media (Thermo Scientific). Human peripheral blood mononuclear cells (Fisher) were cultured in RPMI-1640 and stimulated with 0.2 μg of phytohemagglutinin (PHA; Sigma) for 24 h prior to use. All immortalized cell lines used in this study are routinely checked for mycoplasma and microbial contamination. GeneJuice transfection reagent (Invitrogen) was used for transfections unless otherwise stated.

Molecular Biology

All modifications to Respirovirus reverse genetics plasmids (recSeV and recHPIV3) were performed with PCR amplification of insert fragments using CloneAmp 2×PCR Master mix (Takeda). recSeV-Fushimi-Gluc-P2A-eGFP was derived from recSeV-Fushimi-eGFP (recSeV-eGFP), replacing the eGFP reporter with the previously-reported Gluc-P2A-eGFP reporter cassette from recHPIV3-JS-GlucP2AeGFP. recHPIV3-JS-NanoLuc was derived from recHPIV3-JS-GlucP2AeGFP by replacing the Gluc-P2A-eGFP reporter cassette with the NanoLuc open reading frame from pNL1.1.CMV [Nluc/CMV] (Promega). For minigenome studies, potential resistance mutations were cloned into MeV-Edm and HPIV3-JS L plasmids under T7 control using PCR mutagenesis. All plasmids were sequenced to confirm resistance mutations and sequence integrity. For generation of the baculovirus protein expression system used for RdRP assays, MeV (strain IC-B) L and P genes were codon optimized and cloned into the Fastbac dual vector (ThermoFisher Scientific). MeV L was expressed under the polyhedron promotor and MeV P with a C-terminal His-tag was expressed under the P10 promoter. To generate the baculovirus expression system used for purification of the C-terminal MeV L fragment L₁₇₀₈, MeV L₁₇₀₈ (encoding for aa1-1708 of MeV L, with C-terminal FLAG and His tags) was codon optimized and cloned into the Fastbac dual expression system along with MeV P.

Viruses

recHPIV3-JS NanoLuc was amplified on HeLa cells at 32° C., purified on a 60% sucrose cushion, and further purified on a 25%-65% sucrose gradient. MVi/Alaska.USA/16.00 (MeV-Anc), recCDV-5804p, and recRSV stocks were propagated on Vero-hSLAM, Vero-cSLAM, and HEp-2 cells respectively (MOI)=0.01 pfu/cell). A single freeze-thaw cycle was used to release cell-associated progeny virions. The identity of rescued viruses was confirmed through Sanger sequencing after RT-PCR, using appropriate primers. Progeny titers of MeV, CDV, recHPIV3-JS-NanoLuc, RSV, or SeV virus titers were determined by TCID₅₀ titration on Vero-hSLAM, Vero-cSLAM, Vero-E6, HEp-2 cells, Vero-E6 cells respectively. Titers of clinical of HPIV3 and HPIV1 isolates were determined by TCID₅₀-HA titration on Vero-E6 cells followed by HA assay using guinea pig red blood cells. To generate multi-step virus growth curves, Vero-E6 cells (˜1×10⁵ per well in a 24-well plate format) were infected with recHPIV3 or recSeV as specified (MOI 0.01). Inocula were replace with fresh growth media after 1 hour, infected cell aliquots harvested every 12 hours, and released viral titers determined through TCID₅₀ titration.

HTS and Hit Identification

A total of 141,936 compounds were screened for inhibitory activity against recHPIV3-JS NanoLuc (MOI=0.2). HTS was carried out with automated reading of plates 30 hours after infection. Raw data were imported into the MScreen IT environment and analyzed using data mining tools developed in-house or build into the MScreen package. Hit candidates were defined as compounds with anti-HPIV3 active >2.66×SD in control-dependent mean percent inhibition calculation and >2.1×SD in control-independent mean robust z-score calculation.

Counterscreens

For counterscreens, compound was added in 3-fold dilutions (0.009-20 μM) to 96 well plates seeded with Vero-E6 cells (1.1×10⁴/well), followed by infection with recHPIV3-JS-NanoLuc, recMeV-NanoPEST, recSeV-Fushimi-Gluc-P2A-eGFP, recVSV-NanoLuc, or RSV A2-L19F_{D489E}fireSMASh reporter viruses (MOI=0.2) and automated plate reading 30 hours after infection. To triage reporter-interfering candidates, duplicate plates were infected with recVSV-NanoLuc (MOI=0.2). Cytotoxic concentrations were determined in equivalent serial dilution plates after exposure of uninfected cells for 30 hours to the test article, followed by addition of PrestoBlue substrate (Invitrogen) to quantify cell metabolic activity. Four-parameter variable slope regression was used to determine EC₅₀, EC₉₀, and CC₅₀ concentrations. To determine potential chemical liabilities and compound promiscuity, hit candidates were processed in silico through PAINS filters (www.swissadme.ch) and aggregation predictions using Badapple (http://pasilla.health.unm.edu/tomcat/badapple/badapple). To measure potential promiscuous activities or chemical liabilities of GHP-88309, dose-response assays were carried out under altered conditions: i) cells in dose-response plates were infected with high amounts of recHPIV3-JS-NanoLuc (MOI=3); ii) to measure non-specific off-target aggregation effects, GHP-88309 was diluted in DMEM containing 10% FBS and 10 mg/mL bovine serum albumin, and the dilutions added to cells after 30-minute preincubation followed by infection; and iii) 3-fold dilutions of GHP-88309 were generated at different media pH values (pH 5.5, 6.5, 8.3). In all cases, luciferase activity was measured after incubation of cells for 30 hours at 37° C. in 5% CO₂ environment. For virus yield-based dose response assays, cells were infected (MOI=0.1) in a 24-well plate format with recHPIV3-JS NanoLuc, recMeV-Anc, recCDV-5804, recSeV-Fushimi-eGFP, HPIV1, HPIV2, or HPIV3 in the presence of serial compound dilutions. Titers of progeny cell-associated (MeV and CDV) or released virions (recSeV-Fushimi, HPIV1, HPIV3) were determined 48 hours after infection through TCID₅₀ titration. The MitoBiogenesis in-cell ELISA (Abcam) was used according to the manufacturer's instructions to measure mitochondrial and cellular toxicity after incubation of HBTECs for 72-hour with GHP-88309. To assess whether mitotoxicity in cultured cells was masked by the Crabtree effect, VeroE6 were grown in galactose media as alternative carbohydrate sources, followed by PrestoBlue-based assessment of metabolic activity as outlined.

Minigenome Reporter Assays.

HPIV3 and NiV derived minigenomes were NanoLuciferase, MeV and CDV minigenomes firefly luciferase. The NiV minigenome was modified to encode a NiV specific P-mRNA editing site inserted at the beginning of the Nanoluciferase open reading frame, ensuring that a functional Nanoluciferase mRNA was only produced after mRNA editing by NiV L. Reporter activities were determined in the presence of three-fold serial dilutions of GHP-88309 starting from 300 μM for NiV, 100 μM for HPIV3 and MeV, and 20 μM for CDV. Inhibitory concentrations were calculated as above.

### Time-of-Addition Variation Studies

Vero-E6 or Vero-hSLAM cells were infected (MOI=2) with recHPIV3-JS-NanoLuc or recMeV-NanolucPEST, respectively. At the specified time points relative to infection, compounds (GHP-88309, JMN3-003, AS-48, or ERDRP-0519) were added to the culture media at a final concentration of 20 μM. Reporter gene expression was measured at 24 hours after infection.

### HPIV3 Metagenomic NGS Library Generation and Antiviral Resistance Analysis

Viral RNA was treated with Turbo DNase I (Thermo Fisher). cDNA was generated from random hexamers using SuperScript III reverse transcriptase, and second strand was generated using Sequenase 2.0. Double-stranded cDNA was purified using Zymo DNA Clean and Concentrator. Sequencing libraries were generated using two-fifths volumes of Nextera XT on ds-cDNA with 20 cycles of PCR amplification. Libraries were cleaned using 0.8× Ampure XP beads and visualized on a 1.2% agarose FlashGel and pooled equimolarly before sequencing on an Illumina MiSeq (1×192 bp run). Raw fastq reads were trimmed using cutadapt (−q 20) (https://doi.org/10.14806/ej.17.1.200). To interrogate potential resistance alleles, a Longitudinal Analysis of Viral Alleles (LAVA—https://github.com/michellejlin/lava) was carried out in a custom analysis pipeline. LAVA constructs a candidate reference genome from early passage virus using bwa, removes PCR duplicates with Picard, calls variants with VarScan, and converts these changes into amino acid changes with Annovar. Sequencing reads for the HPIV3 NGS campaign are available in NCBI BioProject PRJNA561835.

### SeV L Gene NGS

RNA extracts from infected cells were reverse transcribed and amplified by SuperScript III One-step RT-PCR kit (Invitrogen) by primer set of ‘L-amplicon f1’ and ‘L amplicon r8’, amplifying 1155-4266 (counted from L gene initiation codon) nucleotides of the L gene. Using this as a template, nested PCR was done to create 8 amplicons (400-450 bp) for NGS Amplified 8 amplicons were mixed together and submitted to NGS. DNA library preparations, sequencing reactions, and initial bioinformatics analysis were conducted at GENEWIZ, using throughout NEBNext Ultra DNA Library Prep kit reagents following the manufacturer's recommendations (Illumina). End repaired adapters were ligated after adenylation of the 3′ ends. Pooled DNA libraries were loaded on the Illumina instrument according to manufacturer's instructions and sequenced by MiSeq on a 2×250 paired-end (PE) configuration. Base calling was conducted by the Illumina Control Software (HCS) on the Illumina instrument. Paired-end fastq files were merged by BBTools and aligned to the reference sequence (8 amplicons tandemly connected) using bowtie2. BAM files created by bowtie2 were processed by IGVtools for variant detection.

qRT-PCR Analyses of Viral and Cellular Transcripts

For MeV transcripts, Vero-hSLAM cells were infected with recMeV-Anc (MOI=3) and incubated in the presence of the GHP-88309, ERDRP-0519, or vehicle (at 37° C. for 3-6 hours, or infected with recMeV-Anc (MOI=0.2) and incubated in the presence of control for 12 hours. For HPIV3 transcripts, VeroE6 cells were infected with recHPIV3-JS NanoLuc (MOI=3) and incubated in the presence of GHP-88309 or vehicle at 37° C. for 2-4 hours. Total RNA was extracted at specified time points using Trizol, and subjected to reverse transcription using either oligo-dT, MeV leader specific, or MeV or HPIV3 antigenome-specific primers as specified. Subsequent qPCR used primer pairs specific for a fragment in the MeV leader RNA, MeV antigenomic RNA, MeV P mRNA, MeV H mRNA, MeV L mRNA, HPIV3 N mRNA, HPIV3 antigenomic mRNA or human GAPDH mRNA, respectively, and samples were normalized for GAPDH. To determine induction of type-1 IFN pathways in HBTECs after HPIV3 infection, cells were infected with recHPIV3-JS NanoLuc (MOI=1) and incubated in the presence of GHP-88309 (6 μM) or vehicle at 37° C. RNA was isolated 16 hours after infection, and cDNA generated as above. qPCR used primer pairs specific for a fragment in human ifit1, ifn-α isg15, mx-a, or human gapdh. Mock-infected and compound treated cells were used to determine whether GHP-88309 alone stimulates type-1 IFN pathways. For cytokine profiling of mouse lung tissues, relative ifn-β, ifn-2; acod1, il6, tnf, cxcl15, il12b, ddx60, gbp2, ifit3, isg15, slfn4, and sp100 mRNA concentrations were determined from total lung RNA extracts, generated 3, 6, and 9 days after infection of mice with recSeV-Fushimi-eGFP. Murine GAPDH mRNA as internal standard, and relative mRNA induction was calculated compared to lung tissues extracted from mock-infected animals. Heat maps were generated based on significant differences between vehicle and GHP-88309-treated animals.

In Vitro MeV Polymerase Assay

MeV P and L proteins expressed in a baculovirus/insect cell system (Fastbac dual expression system and SF9 cells) were purified by Ni-NTA affinity chromatography after lysis of cells in 20 mM imidazole, 50 mM NaH₂PO₄, pH 7.5, 150 mM NaCl, 0.5% NP-40 buffer at 76 hours after infection. Proteins were eluted in 250 mM imidazole, 50 mM NaH₂PO₄, pH 7.5, 150 mM NaCl, 0.5% NP-40, followed by buffer exchange to 150 mM NaCl, 20 mM Tris-HCl, pH 7.4, 1 mM DTT and 10% glycerol with Zeba spin columns. Aliquots of P-L hetero-oligomers were mixed in Mg²⁺ or Mn²⁺ buffer with synthetic RNA oligonucleotide templates as specified and rNTPs (lacking GTP) including 0.07 μM of a ³²P-GTP tracer. GHP-88309 was added to reaction mixes as indicated Amplified material was fractionated through SDS-PAGE, and gels developed through autoradiography.

Biolayer Interferometry

Purified MeV L1708 was biotinylated using EZ-Link NHS-Biotin (ThermoFisher). After biotinylation, excess biotin was removed and buffer exchanged to Octet Kinetics buffer (Fortebio) using a Zeba desalting spin column. Purified, biotinylated MeV L1708 preparation were bound to Super Streptavidin (SSA) high-binding biosensors (Fortebio) and dipped into increasing concentrations of GHP-88309 (1.2 μM to 300 μM) followed by dipping into buffer to generate small-molecule binding and dissociation curves.

Photoaffinity Labeling of GHP-88309-016 to MeV L₁₇₀₈

Purified MeV L₁₇₀₈ was incubated with the photoreactive analog (GHP-88309-016 [40 μM]) for 15 minutes prior to activating the crosslinker. The MeV L₁₇₀₈-GHP-88309-016 mixture was placed on ice and exposed to UV light (365 nm) for 30 min, followed by an additional exposure to UV light (254 nm) for 15 minutes. Protein was then harvested using FLAG resin and subsequently incubated with Laemmli buffer at 56° C. for 15 minutes. SDS-PAGE electrophoreses was then performed using Laemmli buffer on 4-15% acrylamide gels. Bands of interest were excised and analyzed by mass spectrometry. Crosslinked peptides were identified by the Proteomics & Metabolomics Facility at the Wistar Institute.

LC-MS/MS Analyses and Data Processing

Liquid chromatography tandem mass spectrometry (LC-MS/MS) analysis was performed by the Proteomics and Metabolomics Facility at the Wistar Institute using a Q Exactive Plus mass spectrometer (ThermoFisher Scientific) coupled with a Nano-ACQUITY UPLC system (Waters). Gel bands containing MeV L₁₇₀₈ were excised, digested in-gel with trypsin and injected onto a UPLC Symmetry trap column (Waters). Tryptic peptides were separated by reversed phase HPLC on a BEH C18 nanocapillary analytical column. Eluted peptides were analyzed by the mass spectrometer set to repetitively scan m/z from 300 to 2000 in positive ion mode. The full MS scan was collected at 70,000 resolution followed by data-dependent MS/MS scans at 17,500 resolution on the 20 most abundant ions exceeding a minimum threshold of 20,000. Peptide match was set as preferred, exclude isotopes option and charge-state screening were enabled to reject unassigned charged ions. Peptide sequences were identified using pFind 3.1.5. MS/MS spectra were searched against a custom database containing MeV L, Sf9, and Baculovirus, and protein sequences. Search parameters include full tryptic specificity with up to three missed cleavages, peptide mass tolerance of 10 ppm, fragment ion mass tolerance of 15 ppm, static carboxamide-methylation of cysteines, and variable oxidation of methionines. To identify GHP-88309-016 crosslinked peptides, mass addition of 261.0902 (MW of GHP-88309-016) was considered for all amino acid residues.

In Silico Docking

Docking studies were performed with MOE 2018.1001, using the Amber10 force field. Homology models of HPIV3, MeV, NiV, and SeV based on the coordinates reported for RSV (PDB 6pzk) were used for docking studies. After protonation and energy minimization, an induced-fit protocol was used to dock the GHP-88309 in the L structures based on resistance data information. For HPIV3, MeV, and SeV, residues E858, E863, S/A866, 11009, T1010, and Y1106 were selected to identify the target site for docking, and resulting docking poses from each polymerase target compared. Homologous residues in RSV and NiV were selected to perform in silico docking into their respective L proteins.

Ex Vivo Metabolic Stability of GHP-88309

To determine stability in plasma, GHP-88309 was incubated in triplicate in pooled human or CD-1 mouse plasma (BioIVT) in glass tubes in a 37° C. shaker-incubator (150 rpm). Procaine and benfluorex served as positive controls and were analyzed in parallel. Aliquots were taken after 0, 5, 15, 30, 60, and 120 minutes incubation time, mixed with 400 μL of internal standard in acidified (0.1% (v/v) formic acid) acetonitrile solution, clarified by centrifugation and supernatants analyzed by LC-MS/MS. To determine stability in liver microsomes, GHP-88309 was incubated in triplicate in glass tubes in 50 mM Tris-HCl (pH 7.5) buffer with 8 mM MgCl₂, 25 μg/mL alamethicin, Phase I and Phase II cofactors (NADPH regenerating system (NRS) and 2 mM UDPGA, respectively) and 0.5 mg total protein pooled mixed gender human liver microsomes (BioIVT) or pooled CD-1 mouse liver microsomes (Xenotech) in a 37° C. shaker incubator (150 rpm). Verapamil served as Phase I positive control, negative controls lacked cofactors and contained 2% (w/v) NaHCO₃ in water instead of NRS. Aliquots were taken after 0, 15, 15, 30, 60 and 120 minutes and processed as outlined above.

All Animal Studies

Standard group sizes were 5 animals each, unless otherwise stated. Power calculation (P<0.05; 80% power) revealed that this sample size will detect differences in mean virus titer >0.71 logarithmical units Animals were assigned to the different study groups randomly; no blinding of investigators was performed other than for histopathology scoring of lung sections.

Pharmacokinetics Analysis in Mice

All in vivo experiments were carried out in eight-week-old female 129x1/SvJ mice (Jackson Laboratory); as a minimum, animals were monitored once daily. Single-dose PK studies were carried out after i.v. and oral substance administration. For i.v. dosing, GHP-88309 was formulated in 28% PEG200, 5% dimethylacetamide, 67% 30% HPB cyclodextrin in sterile PBS at 1.25 mg/ml. For oral dosing, compound was formulated in 1% methylcellulose in water for a homogeneous suspension of up to 18.76 mg/ml. Mice were administered either 5 mg/kg via tail vein injection, or 50 mg/kg or 150 mg/kg via oral gavage, followed by blood collection after 0, 0.083, 0.25, 0.5, 1, 2, 4, 8 and 24 hours (i.v. group) or 0, 0.25, 0.5, 1, 2, 4, 6, 8 and 24 hours (per oral group). Plasma was prepared from whole blood (2000×g, 5 minutes, 4° C.) within 15 minutes of blood collection and samples stored at −80° C. until analysis by LC-MS/MS. Calibration curve range was 10-10,000 ng/ml, samples above quantitation limit were diluted with commercial CD-1 mouse plasma and retested. Quality control samples were interspersed throughout the run, and no chromatographic interferences were detected in blank CD-1 or 129x1/SvJ mouse plasma. For multi-dose PK studies, mice received GHP-88309 orally at 150 mg/kg dose concentration in a b.i.d. regimen for five days. In the first 4 days of dosing, blood samples were collected 0.5 hours (Cₘₐₓ) and 11.5 hours (trough) after the morning dose. On day 5, a full PK study (lacking the 6-hour time point) was performed after the morning dose as outlined. Blood samples were processed and analyzed as above. To determine GHP-88309 organ distribution, mice were administered a single oral dose of GHP-88309 at 150 mg/kg. Selected organs (brain, lung, spleen, kidney, liver, and heart) were extracted 90 minutes after dosing, flash frozen in liquid nitrogen, and stored at −80° C. until further analysis by LC-MS/MS as described.

Determination of Neutralizing Antibody Titers

Plasma was prepared as above, heat inactivated, serially diluted (2-fold steps) in serum free DMEM, mixed with 100 TCID₅₀ units of recSeV-Fushimi-eGFP, and incubated for 1 h at 25° C. Mixtures were transferred to Vero-E6 cell monolayers and virus neutralization measured after three days by visualization of GFP positive infected cells. Equal amounts of recSeV-Fushimi-eGFP in FBS, DMEM containing 7.5% FBS, and serum-free DMEM served as controls. Each blood sample was tested in two technical repeats.

Efficacy Studies in the SeV Mouse Model

Unless otherwise stated, group sizes were 5 animals each, challenge virus was recSeV-Fushimi eGFP, administered intranasally at 1.5×10⁵ TCID₅₀ units in 50 pI of PBS. Prophylactic treatment with GHP-88309 commenced 2 hours before infection, therapeutic treatment was started 48 hours after infection. For all treatment studies, drug was administered per oral gavage in a b.i.d. regiment at 150 mg/kg body weight. Infected mice were monitored twice daily for clinical signs (body weight loss, body temperature change, overall composure). Lung and trachea viral titers were determined in groups of mice on days 3, 6, and 9 after infection through TCID₅₀ titration of tissue homogenates on Vero-E6 cells. Tissue samples for qRT-PCR were preserved in RNAlater until analysis. For rechallenge studies, GHP-88309 treatment after the primary infection was discontinued after 9 days, followed by resting of animals until 28 days after the primary infection and intranasal reinfection with an 1.5×10⁵ TCID₅₀ of recSeV-Fushimi eGFP. A control group of SeV-naïve mice was infected equally at the time of rechallenge. Mice received no treatment after the second infection, blood was collected on study day 21 and 32 to determine neutralizing antibody titers. For infection with drug-resistant recSeV, animals were infected intranasally as above with the engineered, resistant recombinants or the genetic parent virus, and clinical signs monitored as above. Studies were terminated on day 14 after infection, when no clinical signs were detected and animals infected with the parental recSeV had succumbed to the disease.

Histopathology Scoring

Intact lungs were fixed in formalin, embedded in paraffin, sectioned and stained with hematoxylin and eosin. Blinded samples were scored according to the matrix: 0, no change compared to lungs of uninfected mice; 1, minimal signs of immune cell infiltration; 2, mild inflammation and/or immune cell infiltration; 3, moderate inflammation and/or immune cell infiltration, slight thickening of bronchioles; 4, pronounced inflammation and/or immune cell infiltration, moderate thickening of bronchioles and immune infiltration around airways and blood vessels; 5, severe inflammation and/or immune cell infiltration engulfing up to 50% of lung lobe, severe cuffing/thickening of bronchioles and immune infiltration around airways and blood vessels; 6, severe inflammation and/or immune cell infiltration occurring engulfing >50% of lung lobe, severe cuffing/thickening of bronchioles and immune infiltration around airways and blood vessels.

In Vivo Adaptation of recSeV-eGFP in the Presence of GHP-88309

For in vivo adaptation, mice were administered recSeV-Fushimi eGFP or resistant recombinant intranasally at 1.5×10⁵ TCID₅₀ units in 50 pI of PBS. Prophylactic treatment with GHP-88309 commenced 2 hours before infection administered per oral gavage in a b.i.d. regiment at 50 mg/kg body weight. Infected mice were monitored twice daily for clinical signs (body weight loss, body temperature change, overall composure). Mice were harvested 6.5 days after infection and lung viral titers were determined through TCID₅₀ titration of tissue homogenates on Vero-E6 cells. For each recSeV-Fushimi eGFP passaged, a second mouse was infected with 50 μL of passage 1 lung homogenate. Passage 2 mice were harvested 6.5 days after infection and lung viral titers were determined through TCID₅₀ titration of tissue homogenates on Vero-E6 cells. RT-PCR was performed on SeV RNA from tissue homogenates. A PCR fragment (L residues 815-1181) was sequenced to identify any potential resistance mutations arising from in vivo passaging of recSeV-eGFP in the presence of GHP-88309.

To determine susceptibility of in vivo passaged virus, Vero-E6 cells were infected with harvested SeV-eGFP in 96-well format (5×10³ cells/well). Infected cells were incubated with increasing concentrations of GHP-88309 (0.13-100 μM). At 80 hours post infection, GFP signal was quantified using a Cytation5 imaging reader (Biotek). Average fluorescent intensity was determined using an area scan function (49 reads/well; excitation/emission=479/520 nm) using the Gen5 software package (Biotek; v3.05). Infected cells treated with DMSO and cycloheximide were used as negative and positive controls, respectively. Effective concentrations were calculated from dose-response data sets through 4-parameter variable slope regression modeling.

Human 3D-ALI-HBTEC Models

3.3×10⁴ HBTECs were seeded onto 6.5 mm Costar Transwell Cell Culture Inserts (pore size 0.4 μm) and grown submerged in BronchiaLife cell culture medium to 100% confluency. HBTEC Air-Liquid Interface Differentiation Medium (LifeLine Cell Technology) was then added to the basolateral chamber, and media removed from the apical chamber. Cells were grown at ALI for 21 days, emerging 3D cultures washed every 48 hours to remove excess mucus, and transepithelial/transendothelial resistance (TEER) measured using an EVOM volt/ohm meter coupled with an STX2 electrode (World Precision Instruments). Cultures with resistance ≥700 Ω/cm² were used for experimentation. For infection studies, 3D-ALI-HBTECs were apically inoculated with recHPIV3-JS-NanoLuc, HPIV-1-5F6, HPIV3-9R4, or HPIV3-10L3, (5,000 TCID₅₀/well) for 2 hours and washed thrice with media. Treatment with GHP-88309 or volume-equivalent DMSO was administered from the basal chamber. Released virus was collected from the apical chamber every 24 hours. Treatment efficacy was evaluated by TCID₅₀ (recHPIV3-JS-NanoLuc) or TCID₅₀-HA (HPIV-1 and HPIV3 clinical isolates) titration of shed virus.

Confocal Microscopy

3D cultures were fixed with 4% paraformaldehyde-PBS for 20 minutes followed by permeabilization with 0.5% TritonX-100-PBS for 2 hours. Nonspecific protein binding sites were blocked with 5% BSA-PBS for 1 hour. HPIV3 was detected using goat anti-HPIV3 (Abcam) followed by anti-goat Alex-568 (Thermo Scientific). Muc5AC was detected using mouse anti-Muc5AC (Thermo Scientific). ZO-1 (tight junctions) was detected using mouse anti-ZO1 (BD Biosciences; 610966). Anti-mouse-FITC secondary antibody (SantaCruz) was used for both Muc5AC and ZO-1 staining. Anti-β-tubulin-647 antibody (Novus Biologicals) was used for detection of β-tubulin. Membranes were placed on glass slides using a mounting medium supplemented with 0.1 mM 4,6-diamidino-2-phenylindole (DAPI; Molecular Probes), covered with a coverslip, and edges sealed with nail polish. A Zeiss LSM 800 confocal microscope coupled with AiryScan module was used for detection, Zeiss Zen Blue software was employed for image analysis.

Results

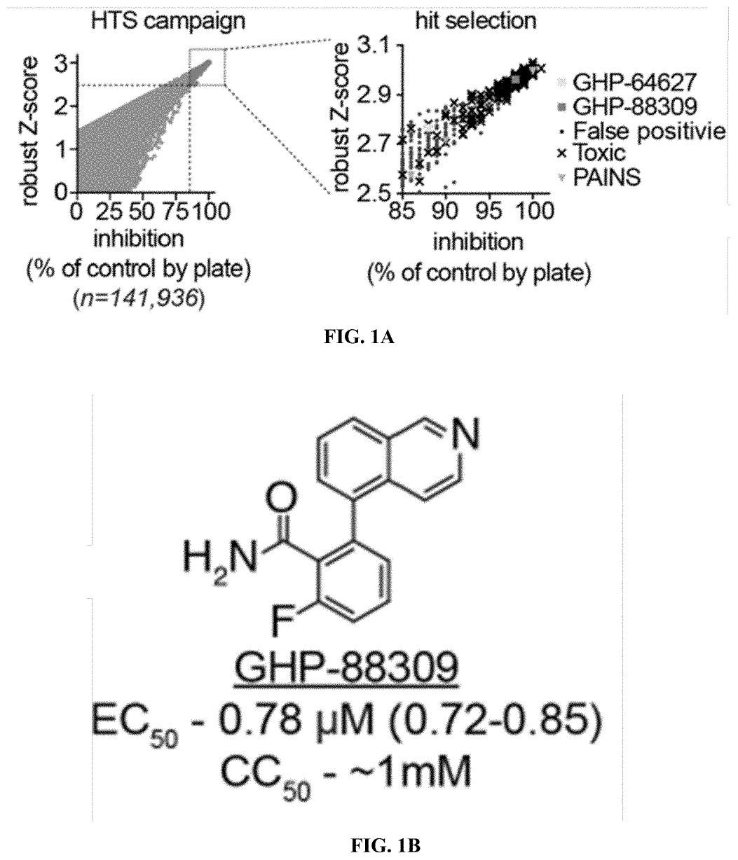

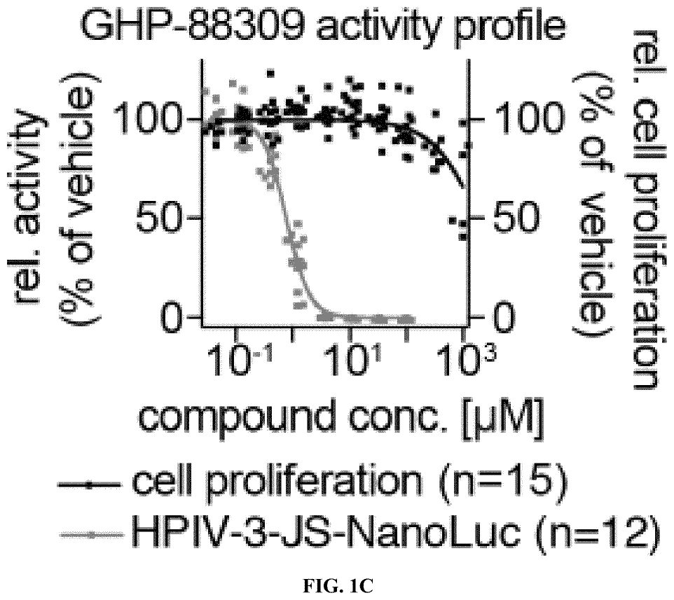

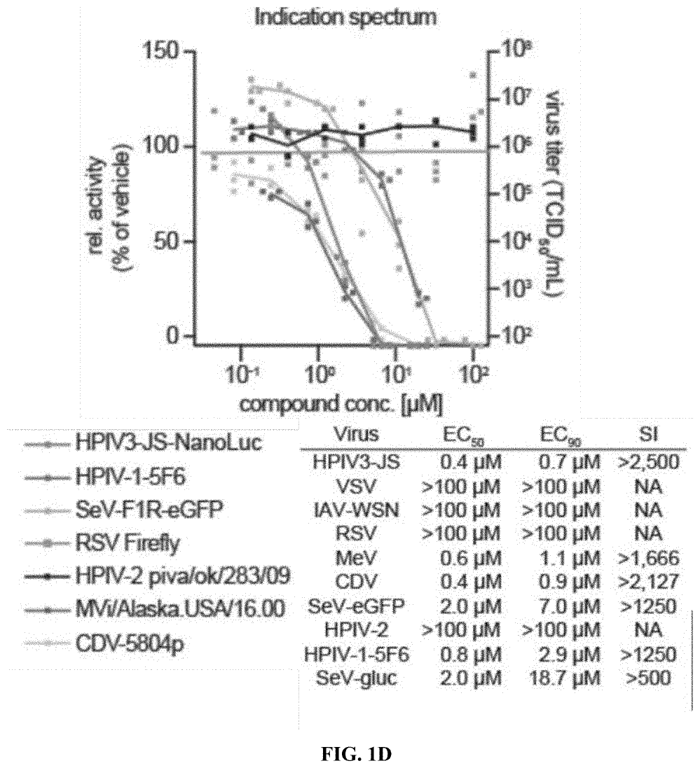

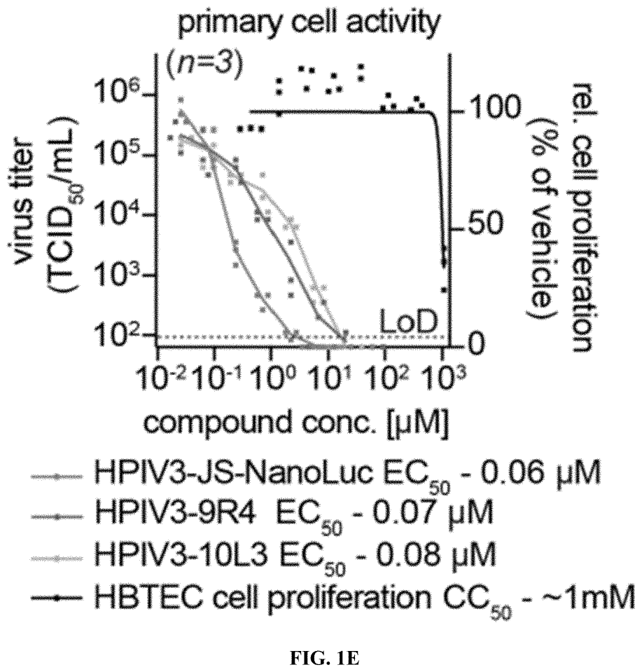

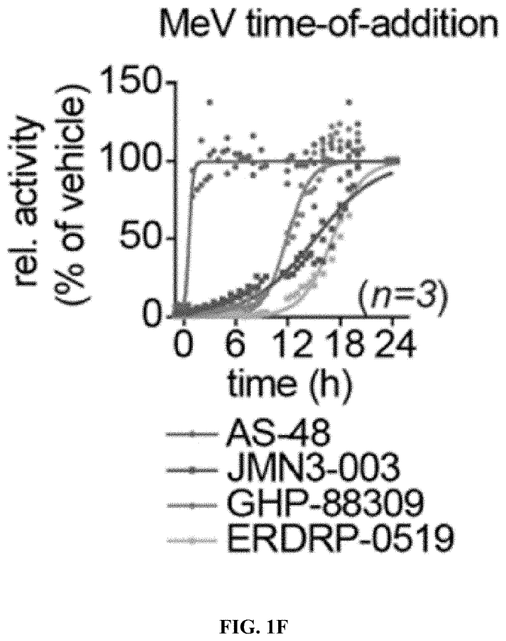

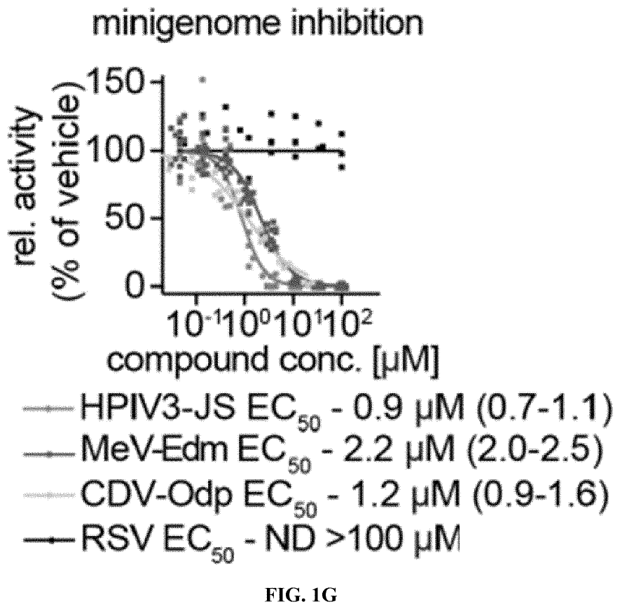

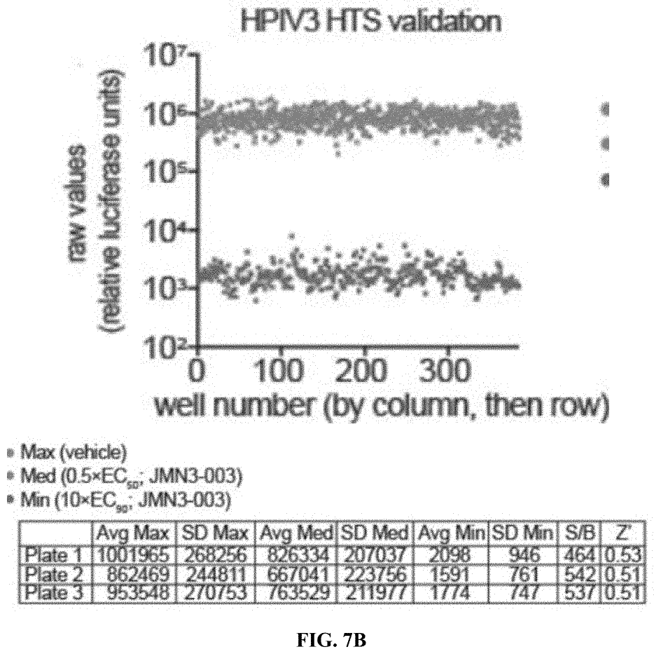

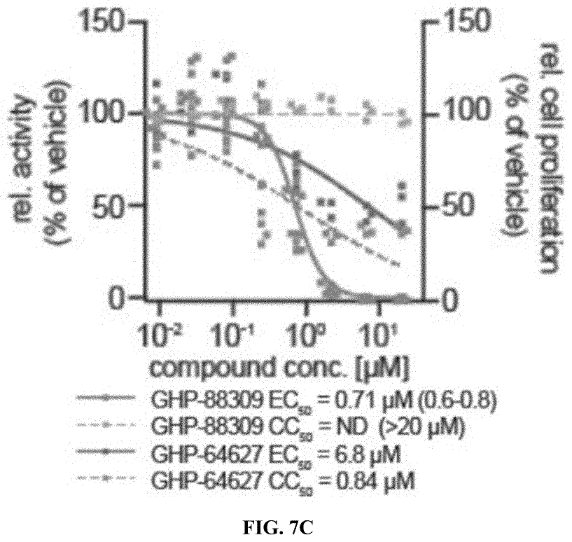

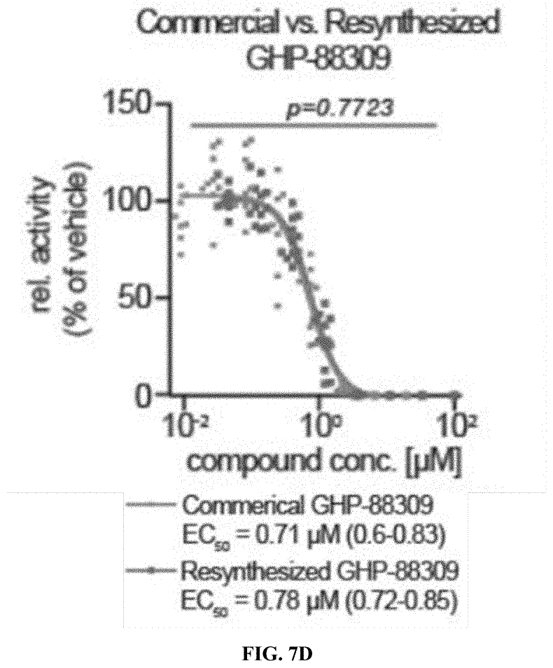

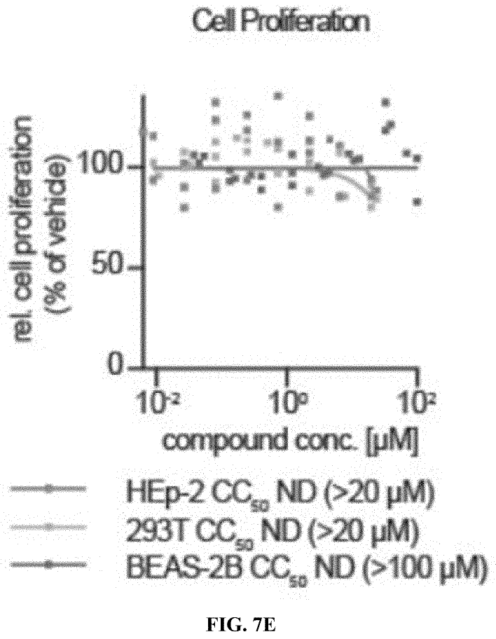

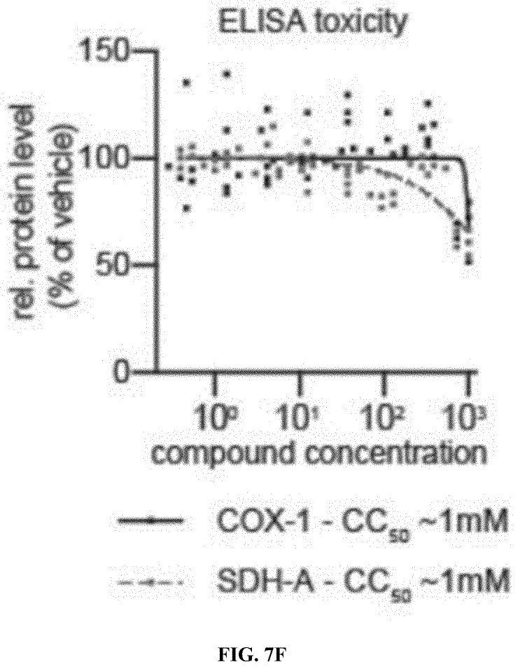

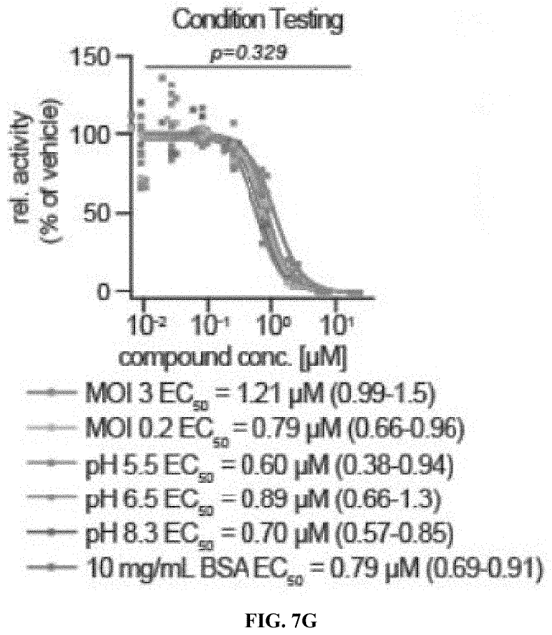

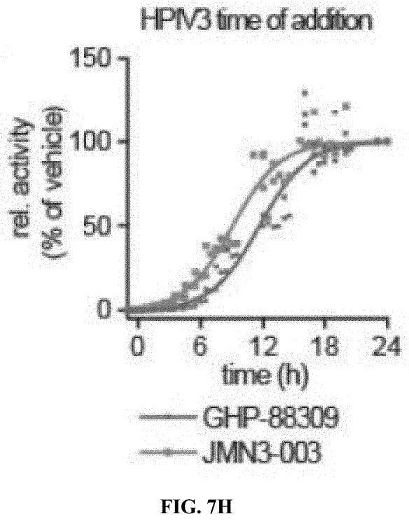

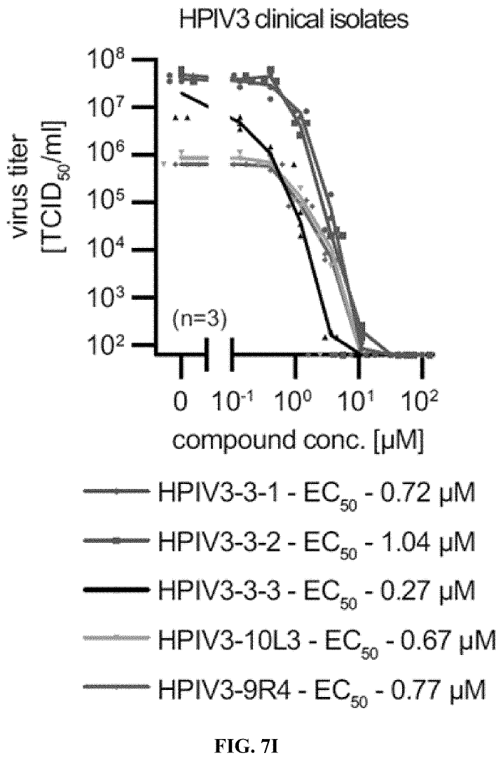

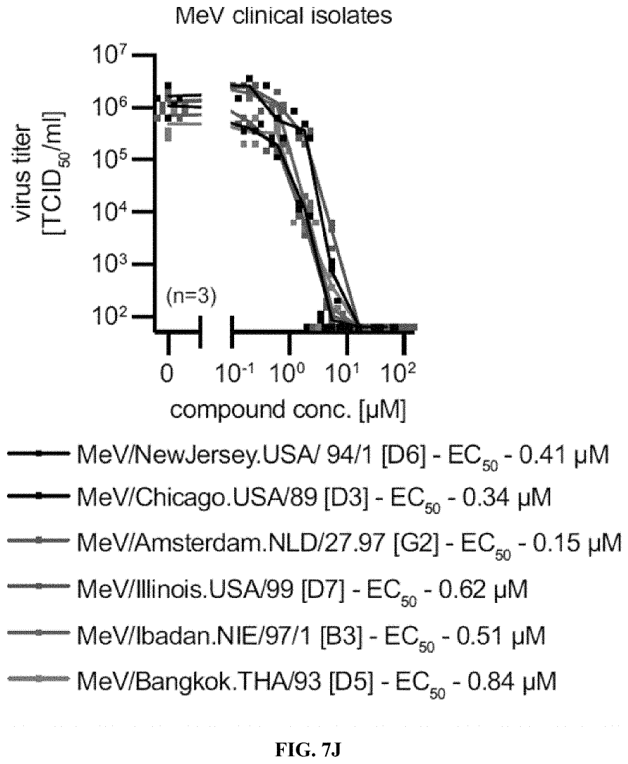

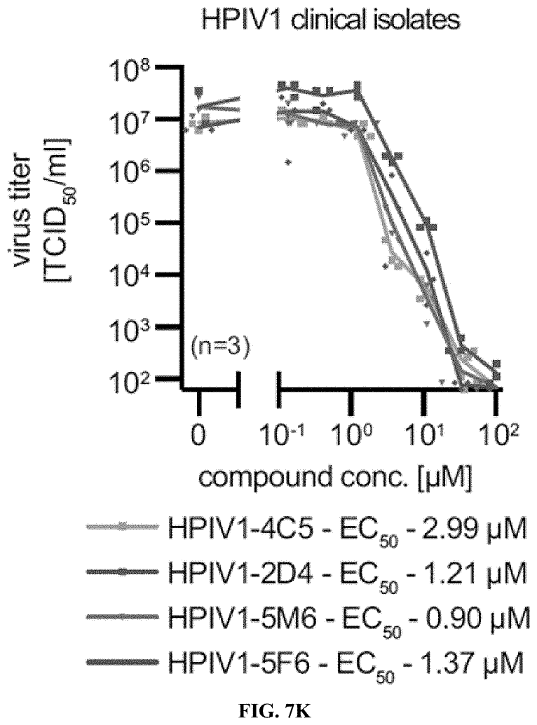

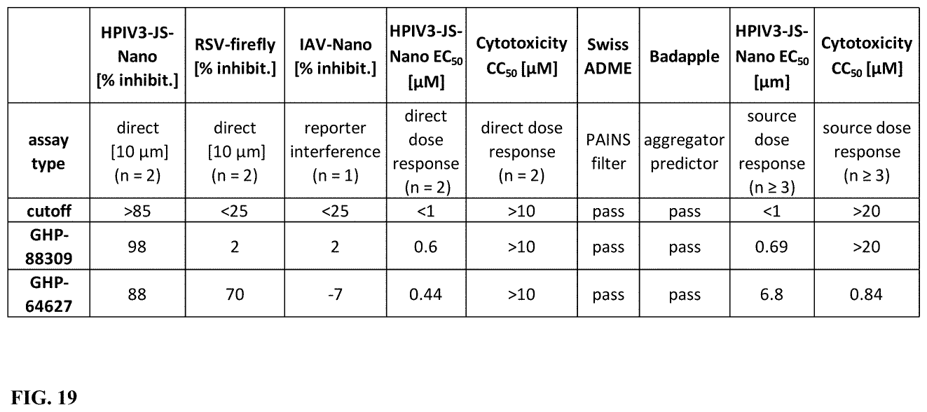

The HTS protocol centered on a recombinant HPIV3 expressing nanoLuciferase (HPIV3-JS-NanoLuc) (FIG. 7A). Fully validated (FIG. 7B), we applied the protocol to screen 141,936 compounds. Combining control-dependent and independent statistical methods for hit identification (FIG. 1A), we admitted 451 compounds to direct and orthogonal counterscreens (FIG. 7C; FIG. 19). Performance requests were EC₅₀<1 μM, CC₅₀>20 μM, lack of reporter interference, and absence of chemotype promiscuity and chemical reactivity. Two compounds, GHP-88309 and GHP-64627, met all criteria and were sourced. GHP-88309 (FIG. 1B) passed subsequent filters, returning active concentrations of 0.4-0.78 μM against recHPIV3-JS-NanoLuc after chemical re-synthesis (FIG. 1C-D), which was statistically indistinguishable (P=0.7233) from the sourced material (FIG. 7D). Multi-day incubation of cells in the presence of up to 1 mM GHP-88309 revealed no cytotoxicity, corresponding to SIs >2,500 in uninfected (FIG. 1E; FIG. 7E-7F) and infected (highest concentration tested 300 μM; FIG. 7G) cultured cells.

Chemotype with Broadened Anti-Paramyxovirus Activity Spectrum

Inhibitory activity of GHP-88309 was not restricted to HPIV3 but included other members of the respirovirus genus tested (HPIV1, SeV) and extended to morbilliviruses (MeV, canine distemper virus (CDV)) (FIG. 1D). Paramyxoviruses of the more distantly related orthorubulavirus genus (HPIV2), pneumoviruses (RSV), and orthomyxoviruses (influenza A viruses) were not inhibited.

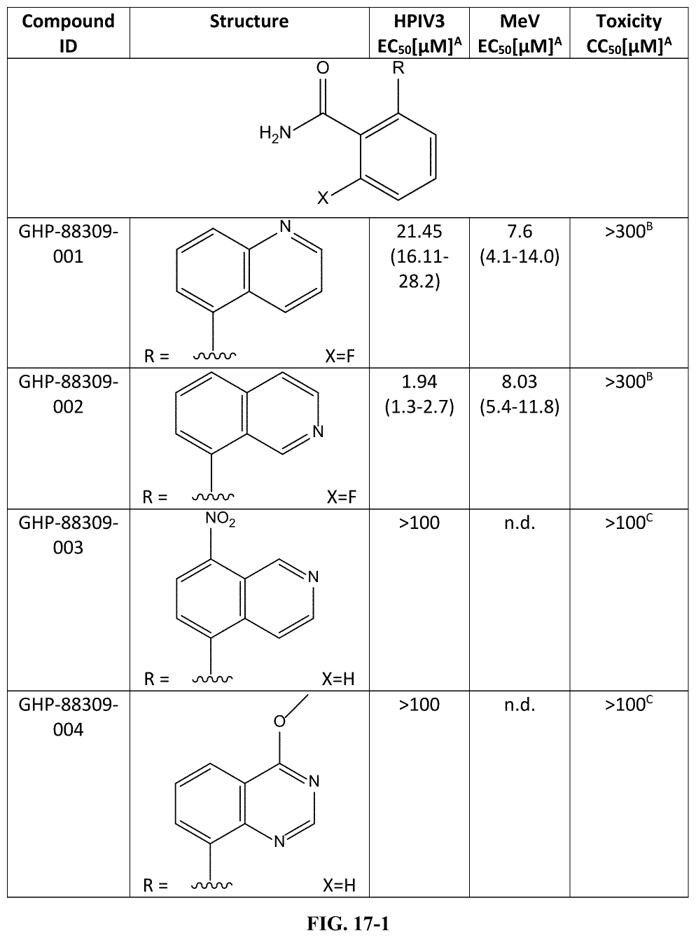

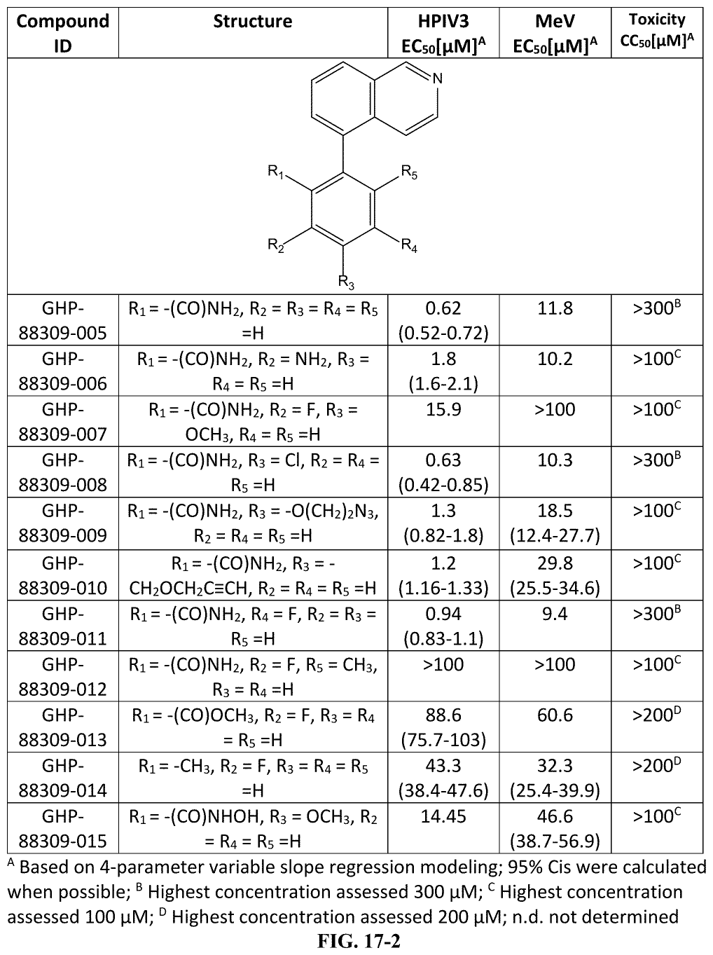

To test whether broad-spectrum activity of GHP-88309 was due to undesirable poly-pharmacologic behavior, the compound aggregation or chemical reactivity was assessed (Auld, D. S., et al., Assay Guidance Manual (2004), Coan, K. E., et al., Mol Biosyst 3, 208-213 (2007), and Habig, M. et al. J Biomol Screen 14, 679-689, (2009)). (FIG. 7G) Only statistically insignificant (P=0.329) changes in antiviral potency (EC₅₀=0.59-1.16 μM) were noted in response to altered pH, varied amounts of virus inoculum, or bovine serum albumin added in high-concentration. Synthetic elaboration of the scaffold revealed a structure-activity relationship (SAR) that was consistent for HPIV3 and MeV targets (FIG. 17) and indicates that GHP-88309 is not a singular hit but the founding member of a distinct antiviral chemotype.

GHP-88309 is Bioactive in Primary Cells and Against Clinical Isolates

When tested in disease-relevant human bronchial tracheal airway epithelial cell (HBTEC) cultures against HPIV3-JS and clinical isolates HPIV3-9R4 and HPIV3-10L3, antiviral potency was increased approximately 5-fold (EC₅₀ 0.07 and 0.08 μM, respectively) in the primary cell/virus isolate system, whereas the low degree of GHP-88309 cytotoxicity remained unchanged (CC₅₀ ˜1 mM) (FIG. 1E). Both isolates had minimal laboratory exposure, having been amplified ex vivo for a maximum of two passages. Expanded testing against an additional three HPIV3, three HPIV1, and six MeV clinical isolates on cultured cells demonstrated consistent antiviral potency across different target viruses and virus strains (FIG. 7I-7K). Time-of-addition (ToA) variation studies using HPIV3 and MeV as targets provided initial insight into the antiviral mechanism of GHP-88309. Previously characterized MeV entry (AS-48) and polymerase (ERDRP-0519) inhibitors and host-directed RdRP blocker JMN3-003 served as references. GHP-88309 ToA profiles overlapped with those of the polymerase inhibitors ERDRP-0519 and JMN3-003 (FIG. 1F; FIG. 7H). Follow-up minigenome reporter assays established for MeV-Edm, HPIV3-JS, and CDV-Onderstepoort revealed specific inhibition of viral RdRP activity by GHP-88309 (FIG. 1G), since an RSV-derived minigenome assay was not affected.

Resistance Hot-Spots Locate to L Protein

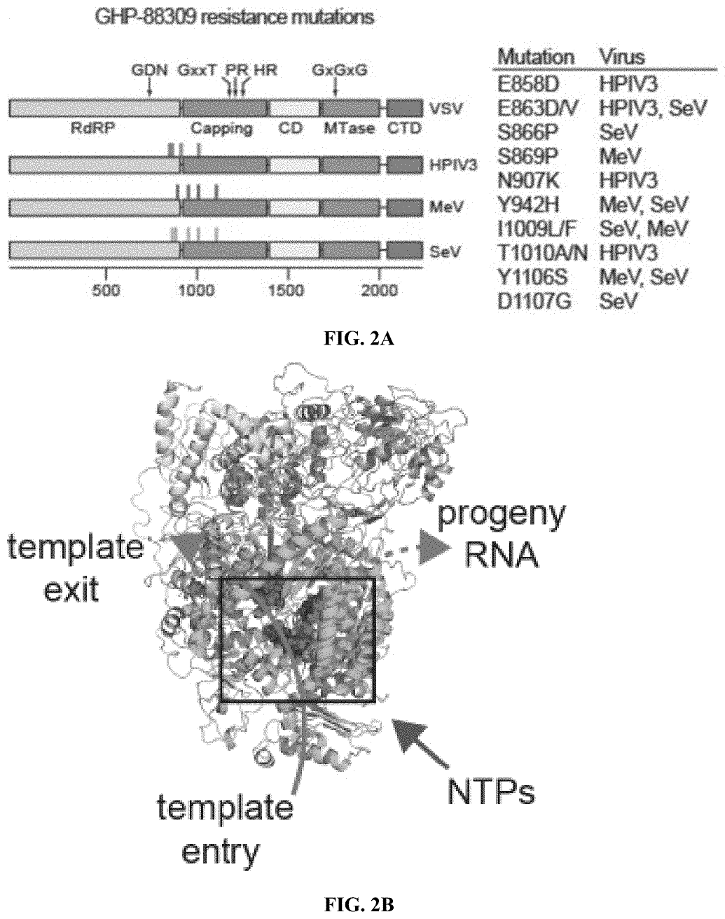

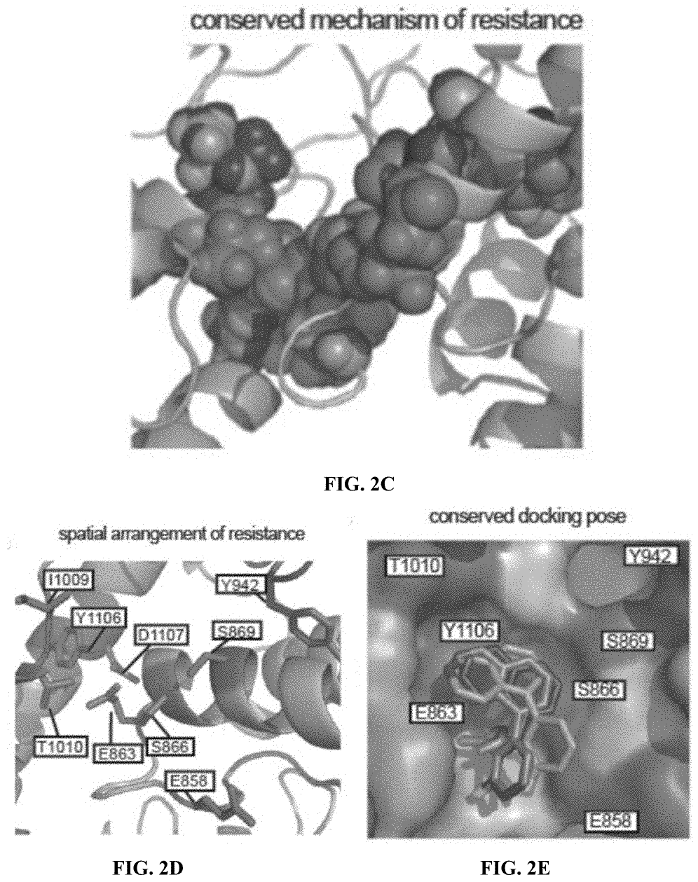

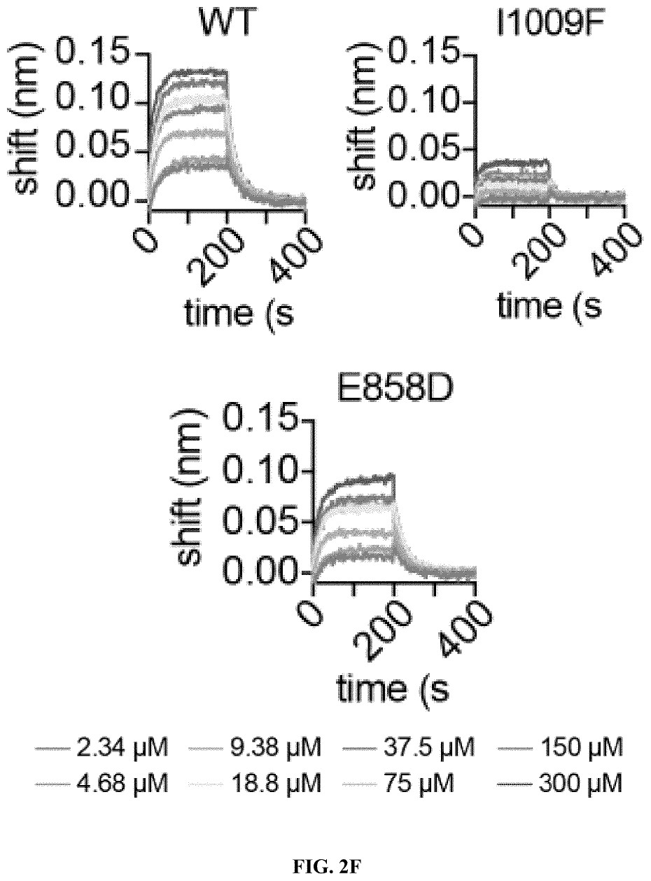

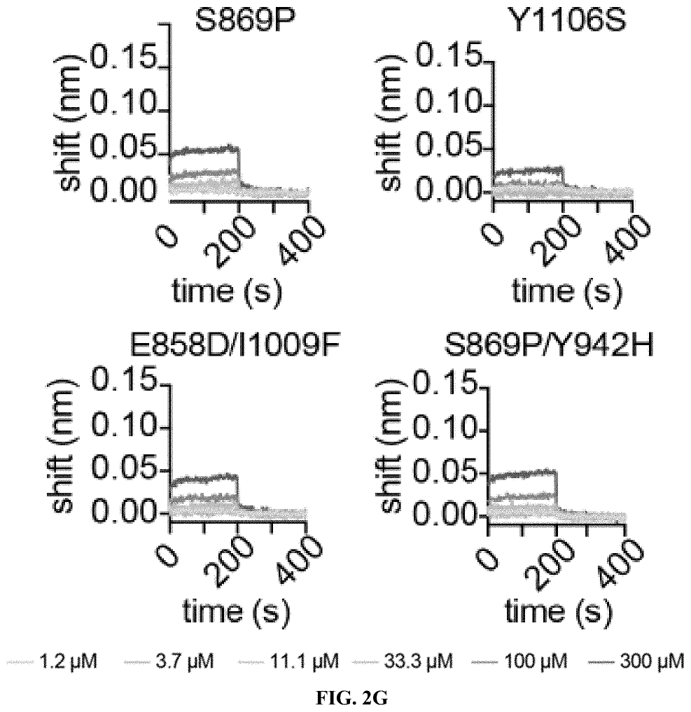

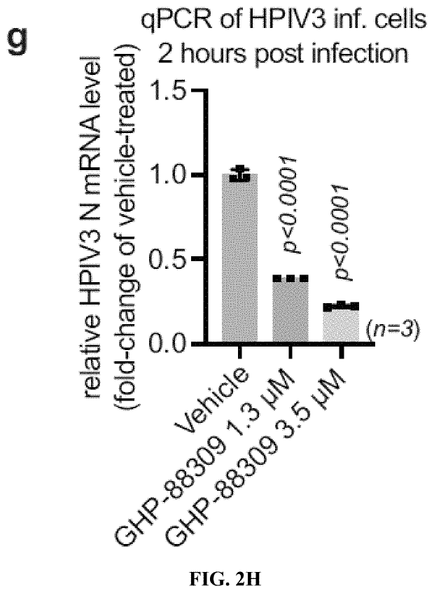

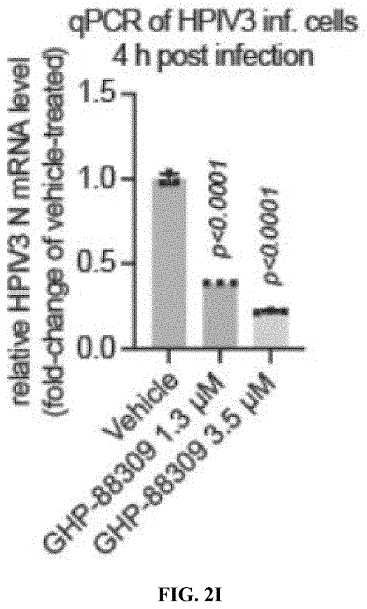

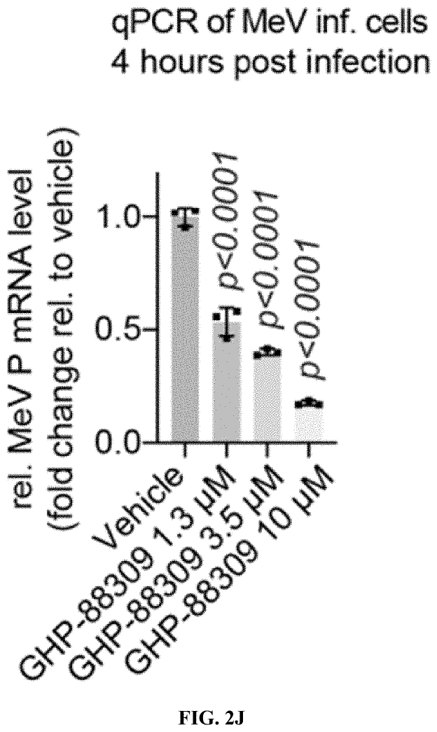

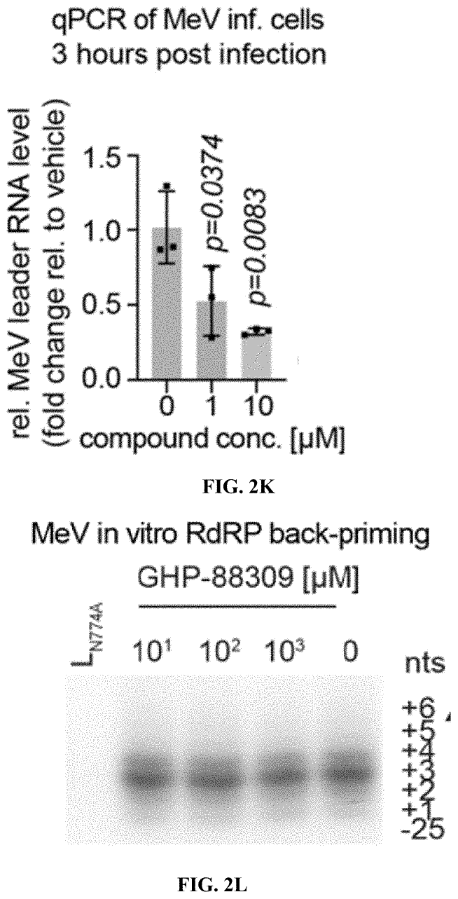

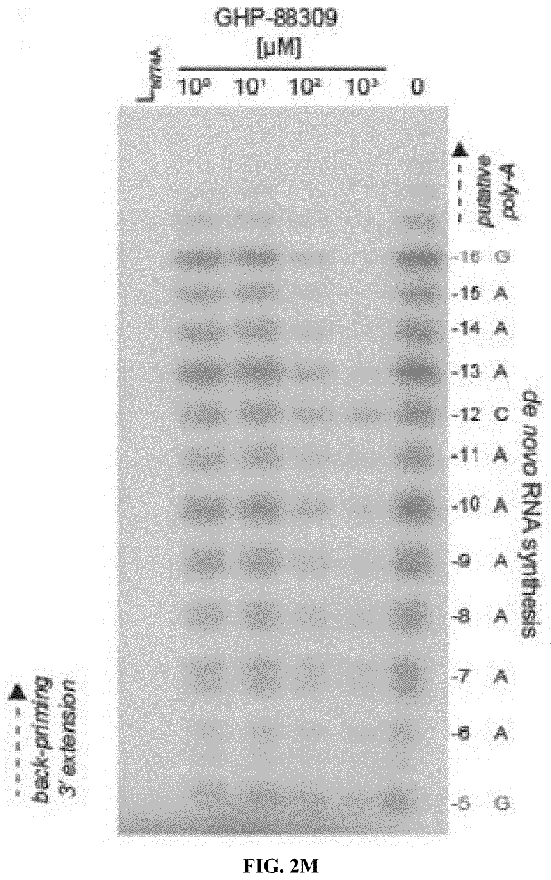

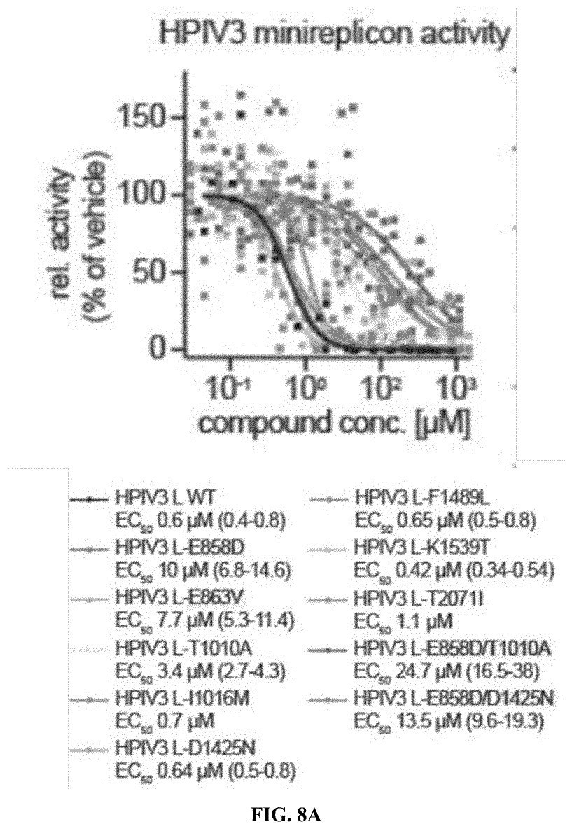

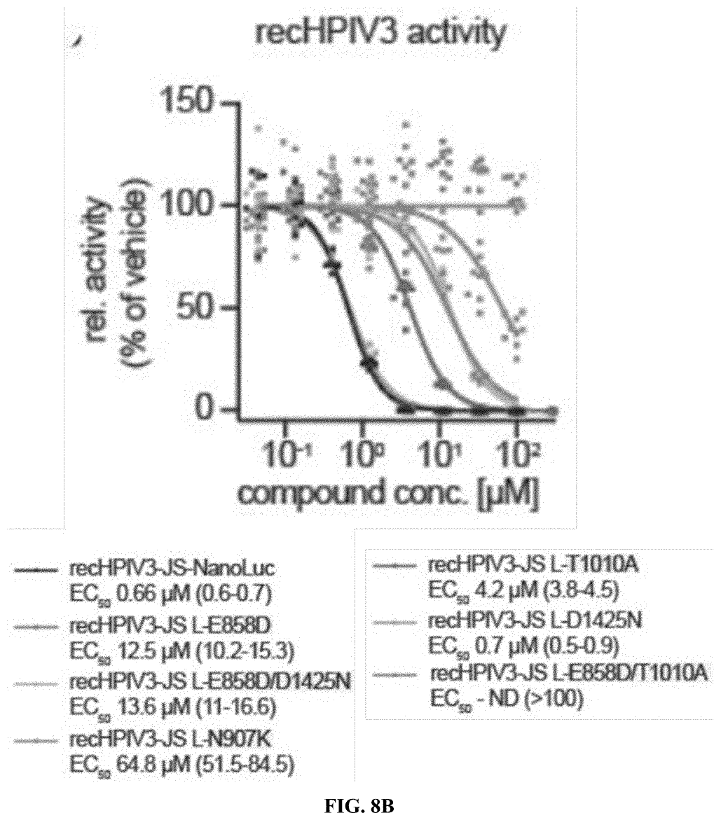

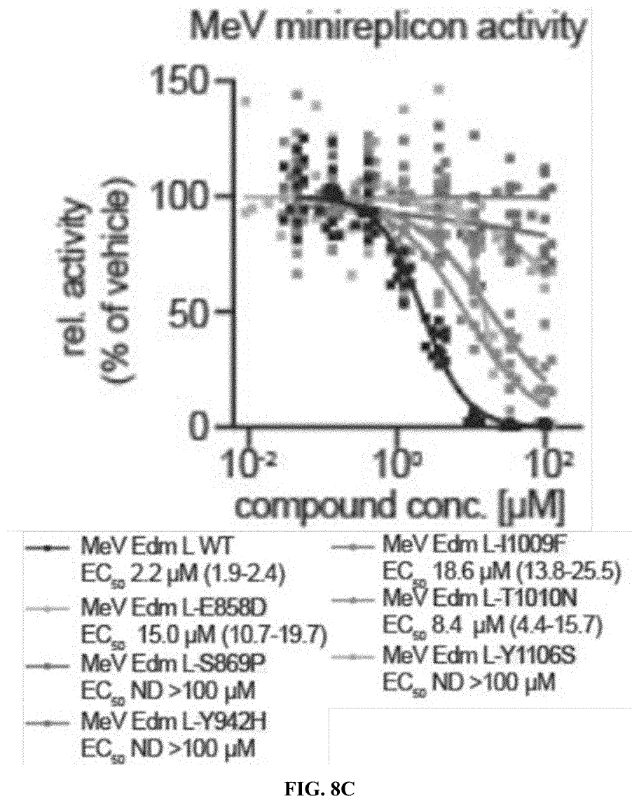

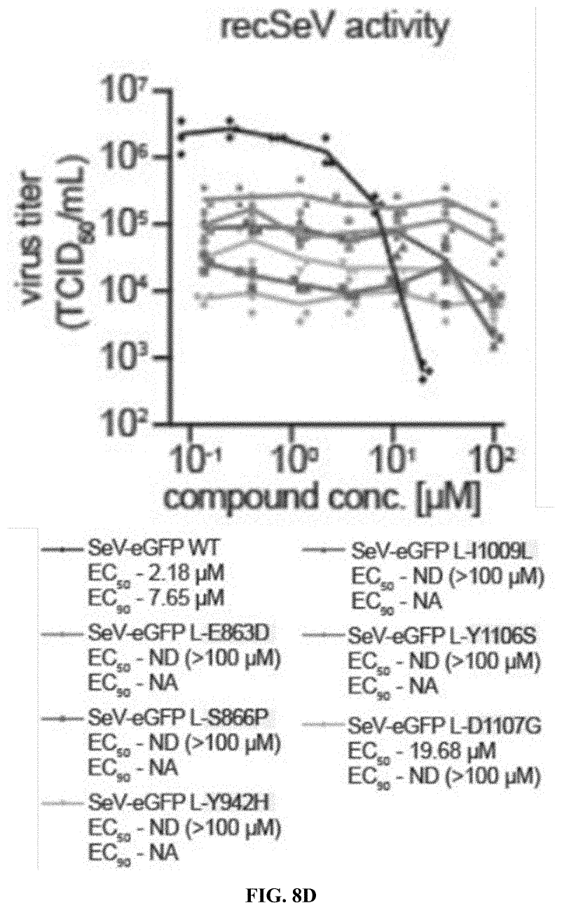

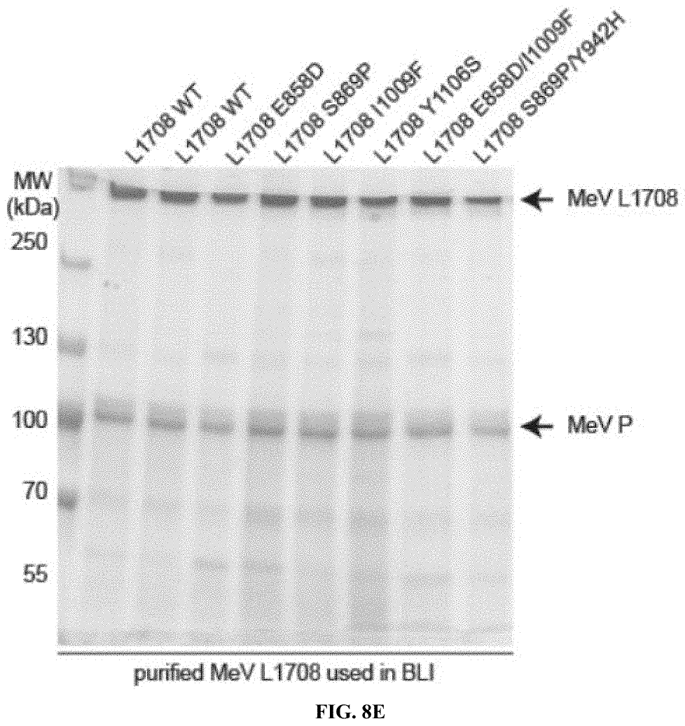

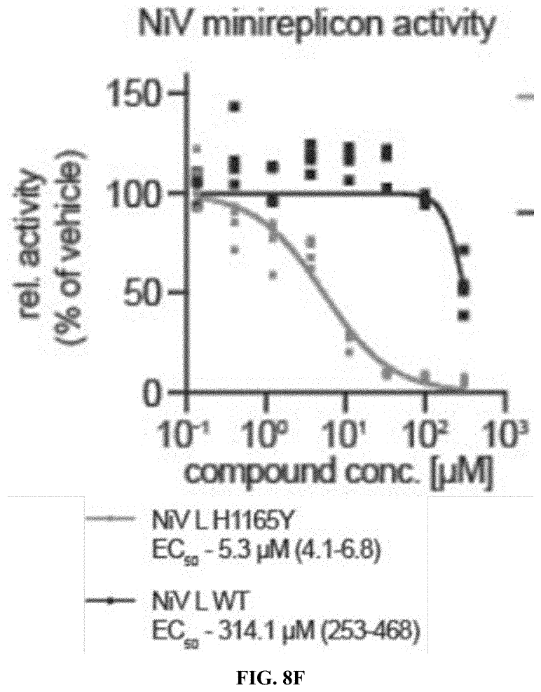

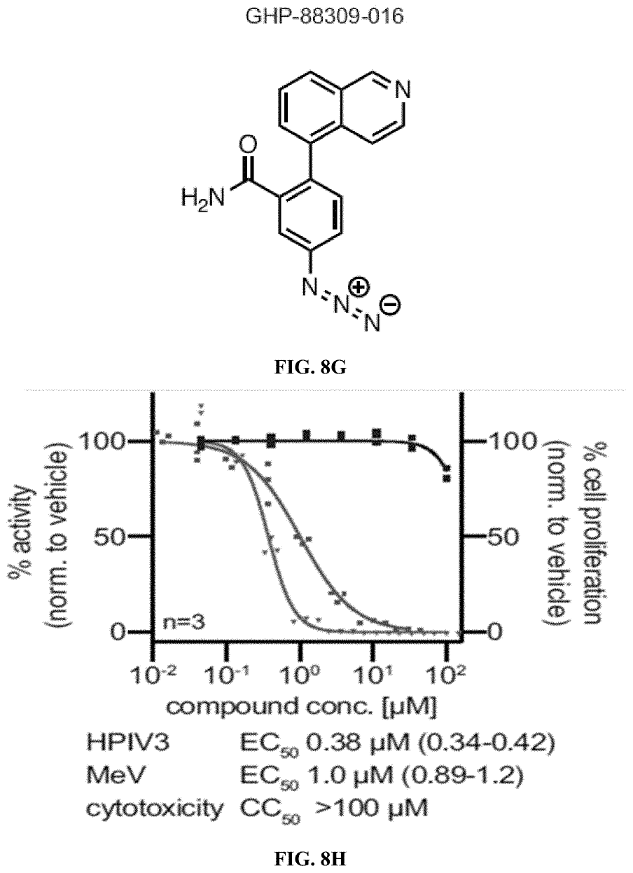

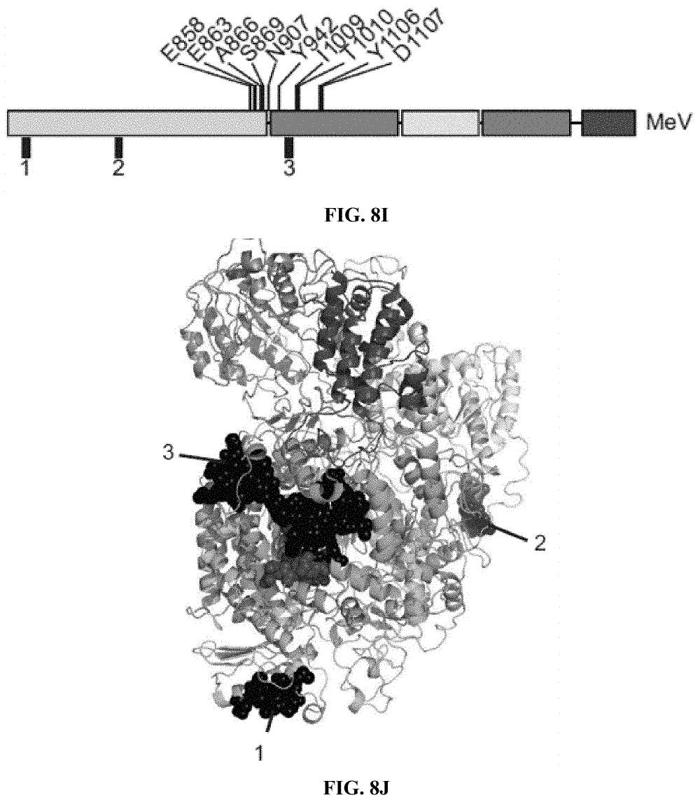

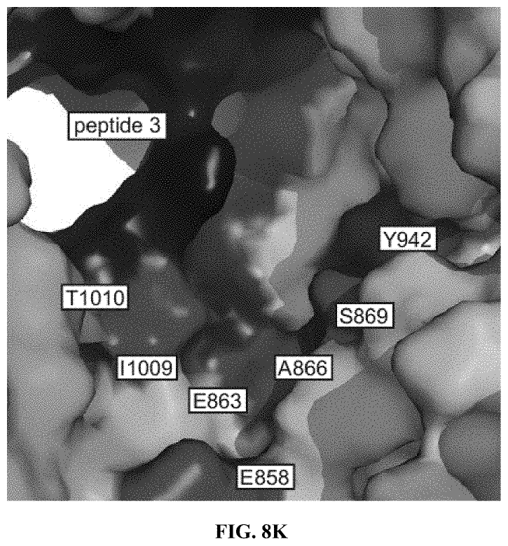

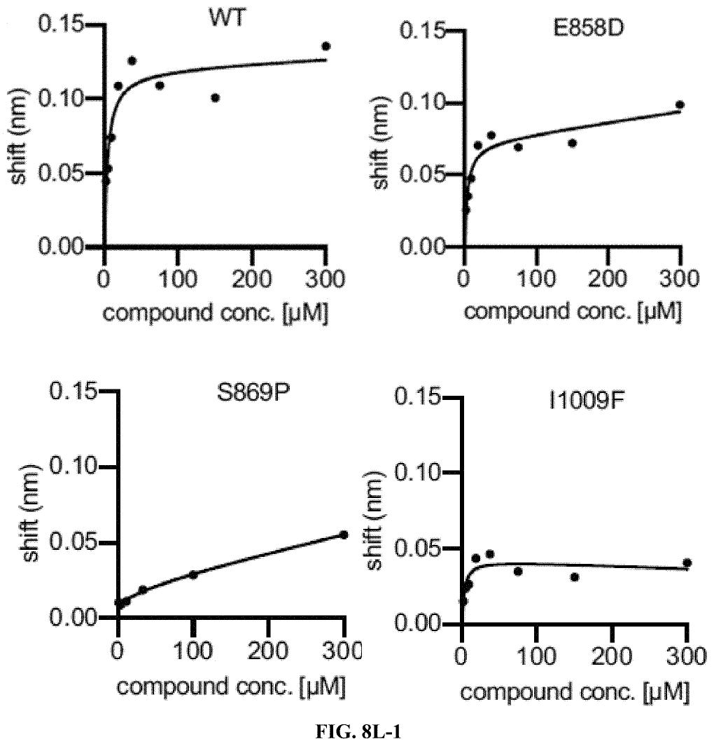

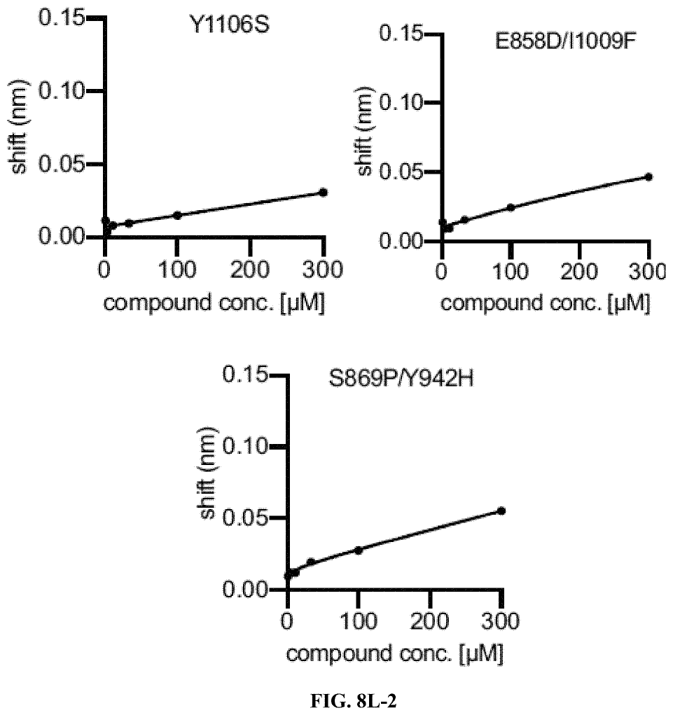

We determined resistance profiles of GHP-88309 for MeV, HPIV3, and—representing the challenge virus of our surrogate small-animal efficacy model—SeV. Dose-escalation viral adaptation (0.5-200 μM concentration range) was carried out in six series per target until viral growth in the presence of compound improved. Sanger and next-generation sequencing revealed mutations in the L proteins of all phenotypically-resistant HPIV3, SeV, and MeV populations (FIG. 2A). Whole genome analysis of resistant HPIV3 populations showed no mutations in other viral protein components associated with the RdRP complex, the nucleocapsid (N) and phosphoprotein (P). Several substitutions affected the attachment and fusion glycoproteins, but these changes reportedly arise from HPIV3 adaptation to immortalized cell lines and were not pursued. An L_{G1036E} polymorphism appeared in several SeV lineages after exposure to GHP-88309 or vehicle, suggesting a natural polymorphism or cell-culture adaptation effect. All other L mutations were rebuilt in respective HPIV3, SeV, or MeV minigenome systems and/or recombinant viruses and specific resistance sites identified (FIG. 8A-D).

To structurally locate confirmed sites, we generated homology models of HPIV3, SeV, and MeV L based on a cryo-electron microscopy reconstruction of the related PIV5 L protein. All validated sites clustered in a conserved L microdomain that lines the interior of the template channel near the intersection of the RdRP and capping domains (FIG. 2A-D), corresponding to the thumb domain of the polymerase complex. This tight and conserved spatial arrangement of resistance mutations suggests proximity to the physical target site of GHP-88309.

Photo-Crosslinking Target Mapping and Extraction of a Docking Pose

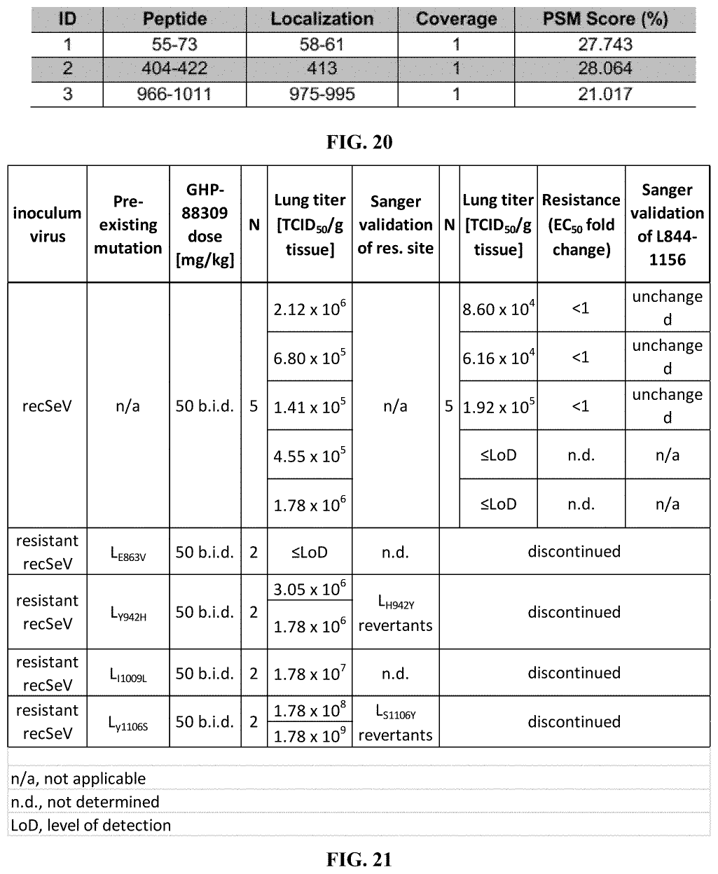

Informed by the emerging SAR, we designed a photo-activatable azide analog, GHP-88309-016 (FIG. 8G), which maintained antiviral activity against HPIV3 and MeV in cell-based assays (FIG. 8H). Using purified MeV L complexes (FIG. 8F), we covalently photo-coupled GHP-88309-016 and identified docking sites through LC-MS/MS. Three distinct peptides contained an additional mass corresponding to the ligand (FIG. 20; FIG. 8I). Localization in the MeV L structural model suggested two of these peptides (FIG. 8J) likely form non-specific cross-links reported for phenylazides such as GHP-88309-016, while residues 992-994 in peptide 3 form part of the central L cavity wall immediately proximal to the resistance cluster (FIG. 8K).

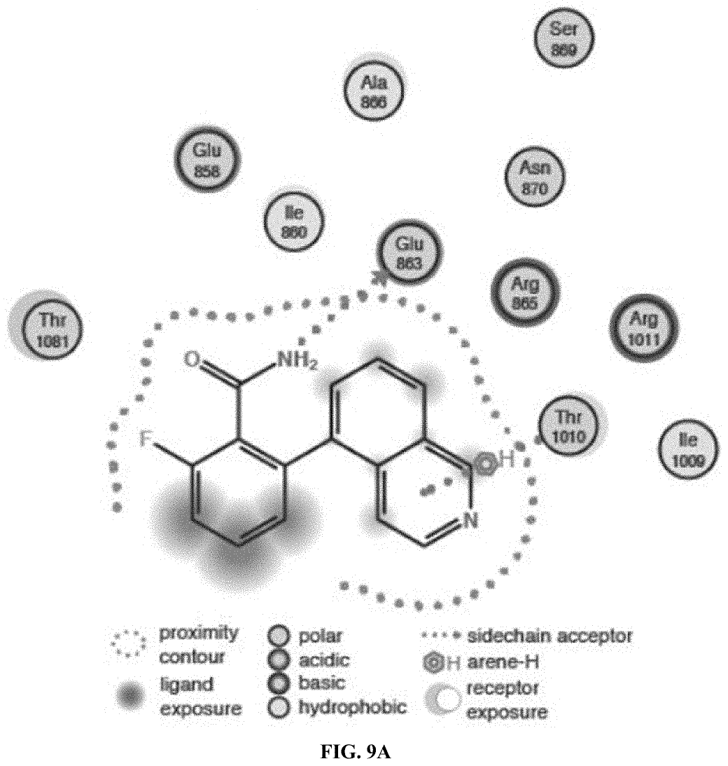

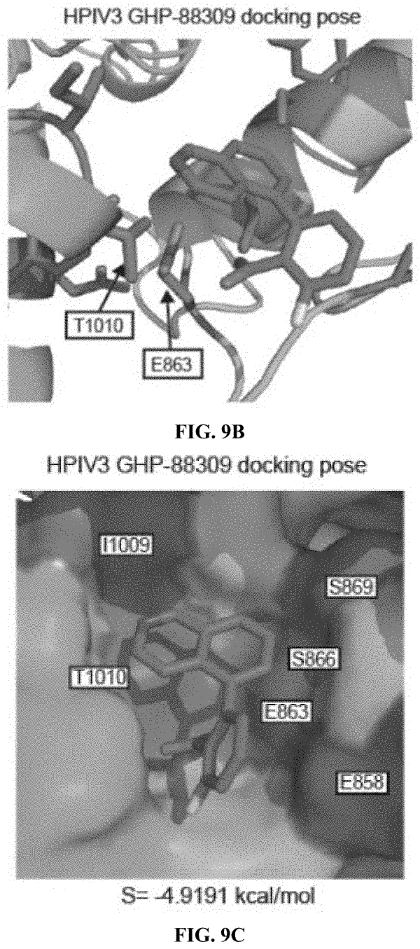

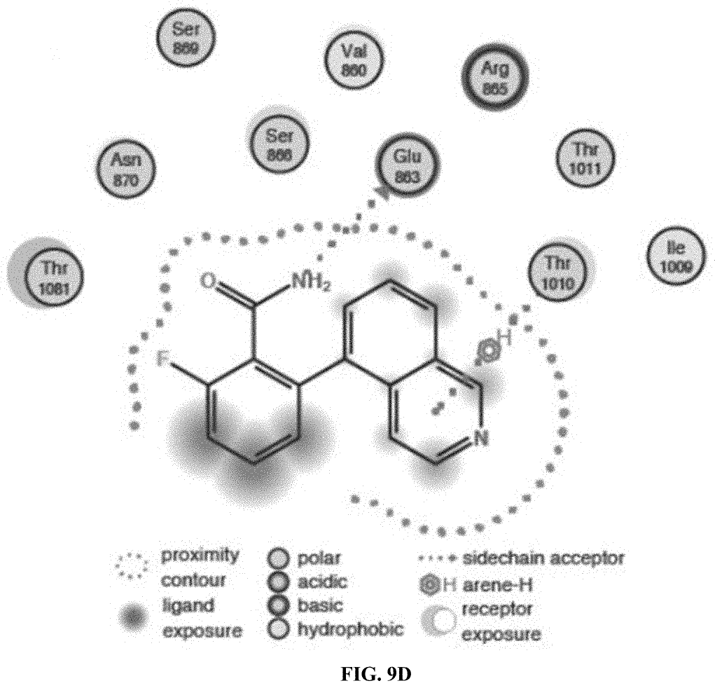

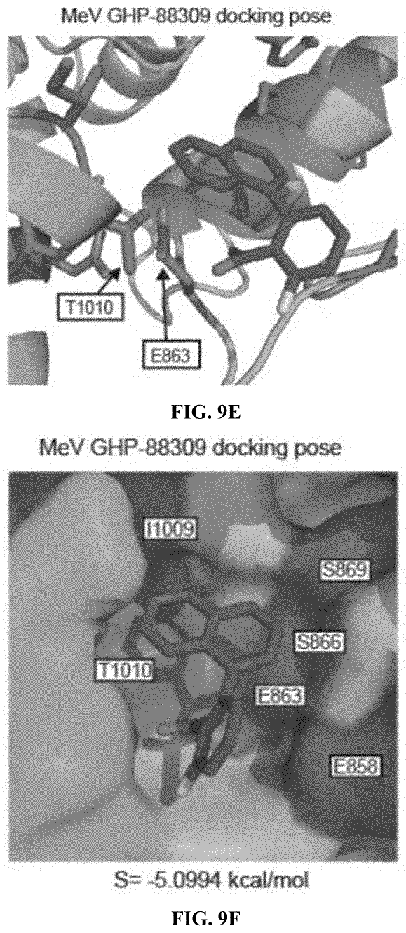

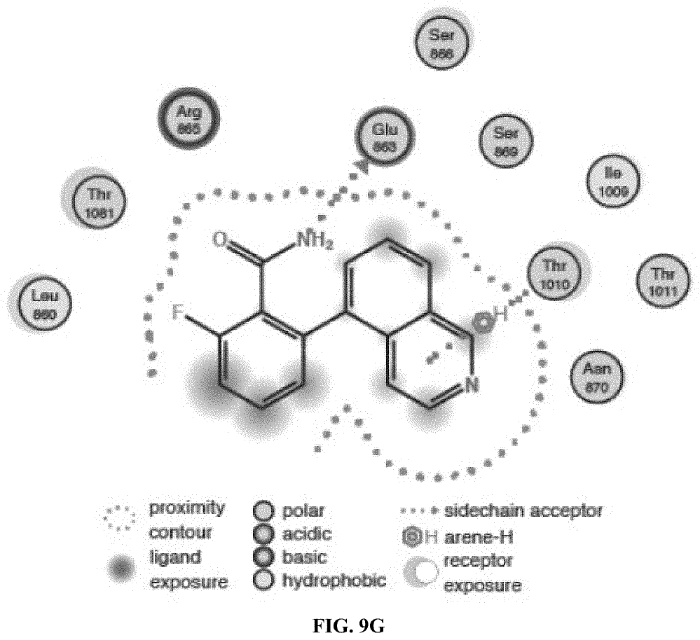

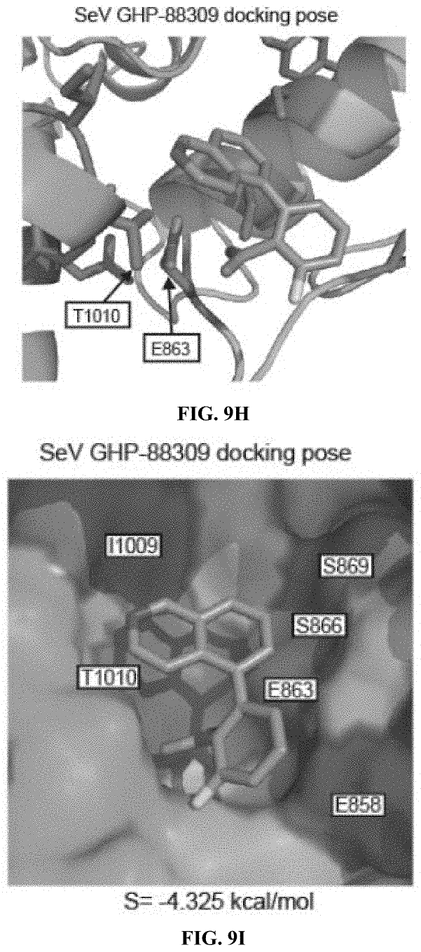

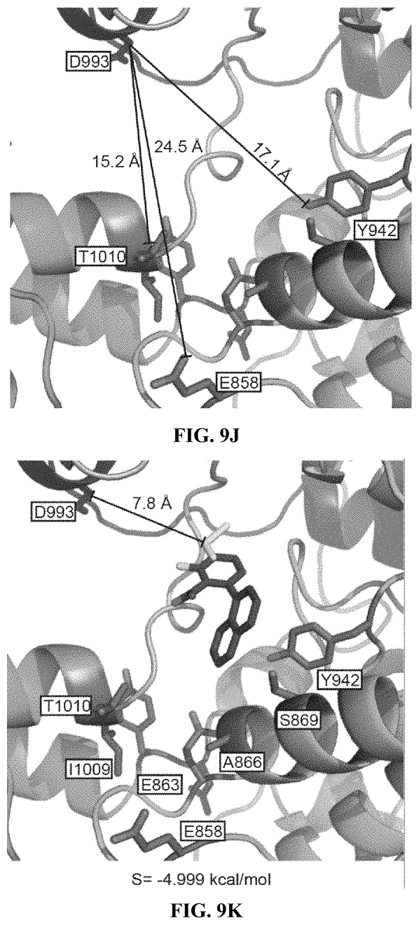

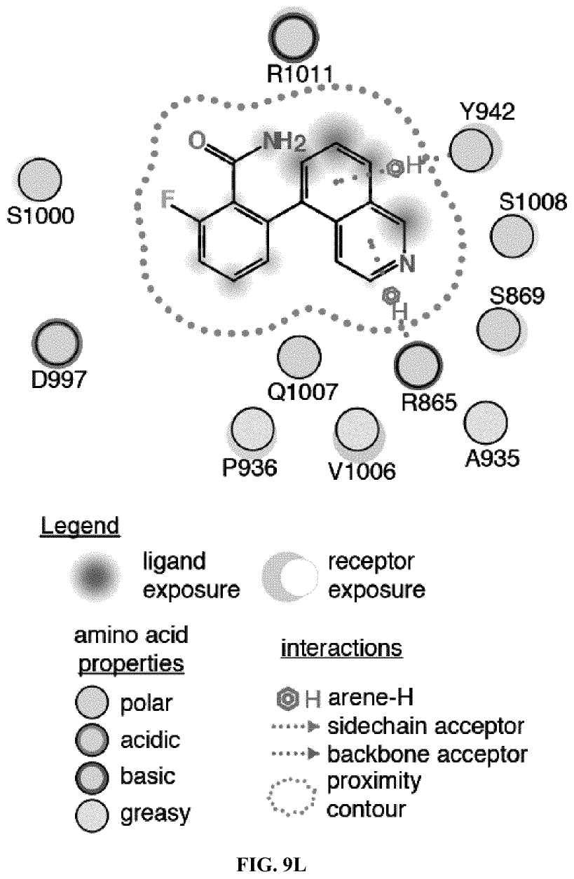

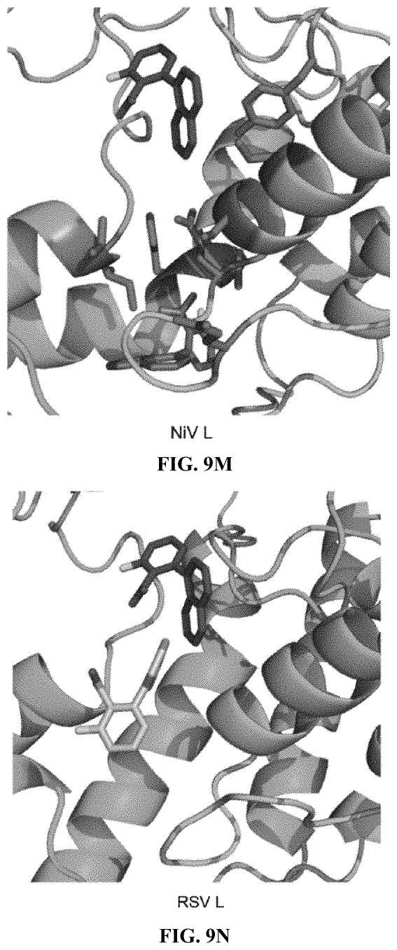

Based on closest mutual proximity (FIG. 9J), we selected residue D993 of peptide 3 and resistance sites E863, A866, 5869, Y942, 11009, T1010, and Y1106 as targets for in silico docking. A conserved top-scoring pose for GHP-88309 and GHP-88309-016 placed the ligands at the interface of the capping and polymerase domains (FIG. 2E), bringing the aryl-azide functional group of GHP-88309-016 within ˜8 Å of residues 992-994 in peptide 3 (FIG. 9K). The isoquinoline ring of GHP-88309 is predicted to form π-hydrogen bonds with L residues Y942 and R865, which are conserved in susceptible viruses, and the benzamide moiety is positioned within 5 Å of residues Q1007 and R1011 (FIG. 9L). This arrangement pharmacologically links the L capping and RdRP domains. Equivalent in silico GHP-88309 docking attempts to L proteins of uninhibited Nipah virus (NiV) and RSV failed to yield comparable binding poses (FIG. 9M-9N).

Resistance Decreases Inhibitor Binding Affinity

Biolayer interferometry (BLI) was used to monitor GHP-88309 affinity for purified MeV L immobilized on biosensors. GHP-88309 binding to standard MeV L reached saturation (FIG. 8L) and showed an affinity (K_{D}) of 6.2 μM, whereas binding to L proteins with bona fide resistance mutations (L_{S869P}, L_{I1009F}, L_{Y1106S}, L_{S869P/Y942H}) was drastically reduced (FIG. 2F; FIG. 2G). Substitution at a respirovirus resistance hot-spot, L_{E858D}, in the MeV L background, yielded only moderate escape. A ˜10-fold difference between EC₅₀ and K_{D} concentrations presumably reflects the conformational heterogeneity of paramyxovirus polymerase preparations, only a subset of which is bioactive and thus represented in the EC₅₀ value.

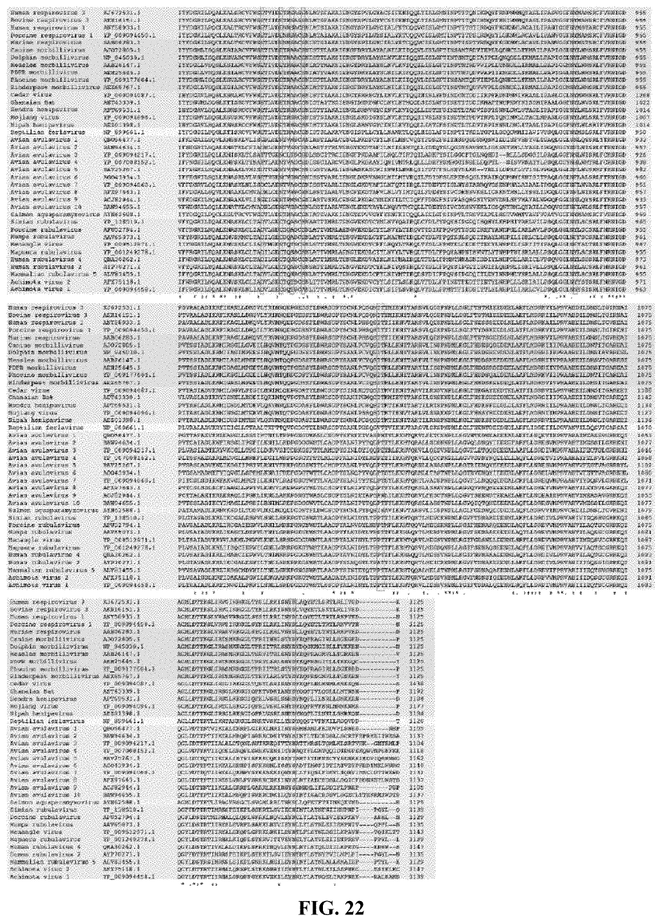

Sequence database searches for HPIV3, HPIV1, and MeV L did not show natural polymorphisms at confirmed GHP-88309 resistance sites. An exception was a laboratory-generated temperature-sensitive HPIV3 vaccine candidate that is attenuated through L_{Y942H} mutation. However, paramyxovirus genera containing sequence variations in the target microdomain, such as the orthorubulavirus HPIV2, were not inhibited by GHP-88309 (FIG. 22). Likewise, NiV L, which features a histidine at position 1156 homologous to the L_{Y1106H} resistance substitution in MeV and SeV, was uninhibited (EC₅₀=314.1 μM; FIG. 8E). Introducing L_{H1156Y} mutation into NiV L conferred susceptibility to GHP-88309 (59-fold lower inhibitory concentration in minireplicon assays; EC₅₀=5.3 μM) (FIG. 8E), indicating that the compound target site emerged structurally conserved from paramyxovirus evolution and acquired only isolated point mutations across different genera.

GHP-88309 Impairs Initiation of RNA Synthesis

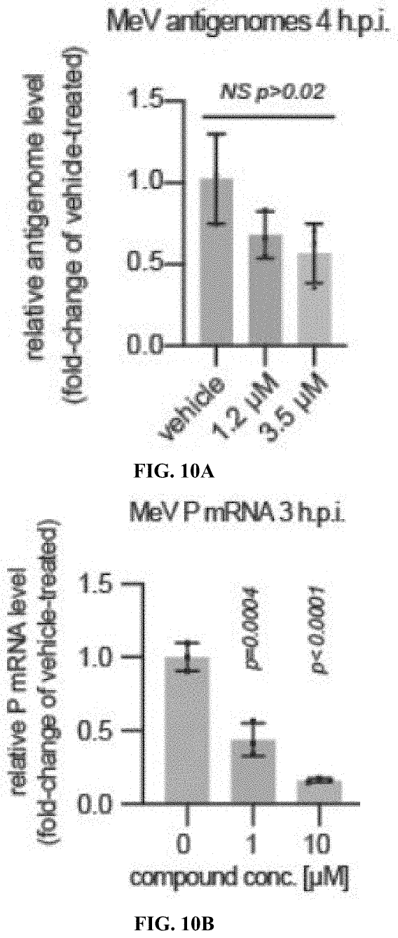

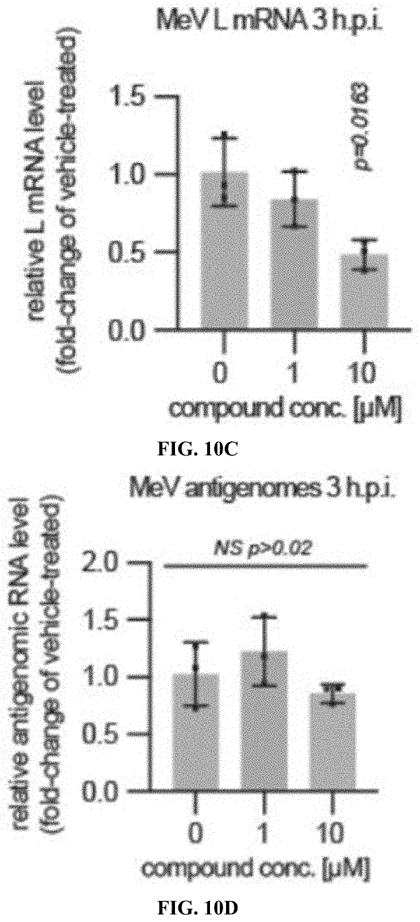

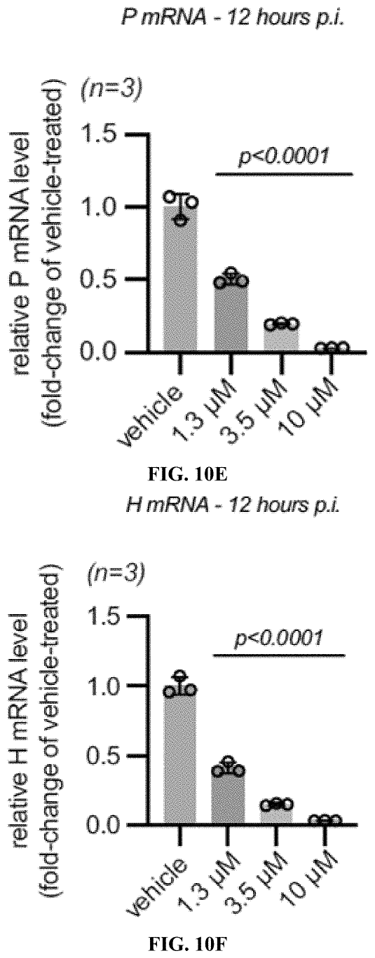

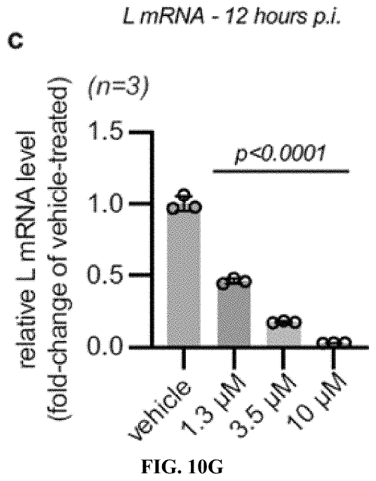

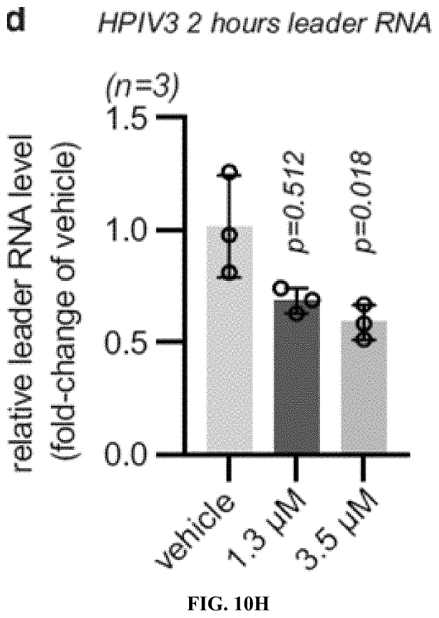

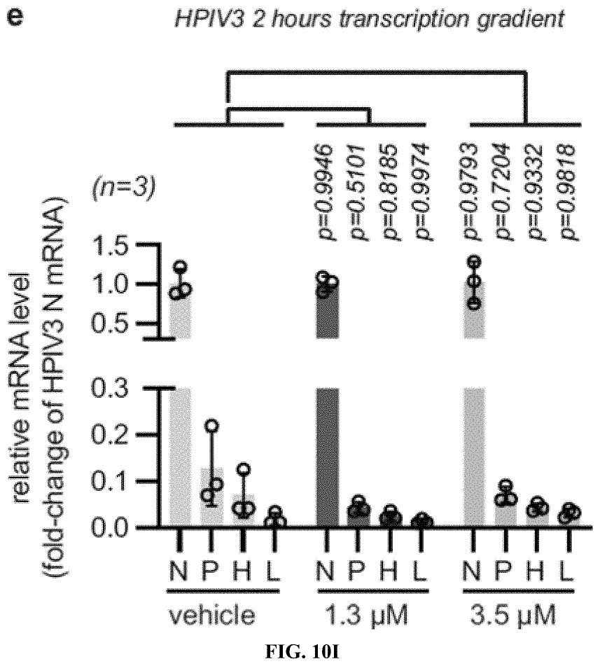

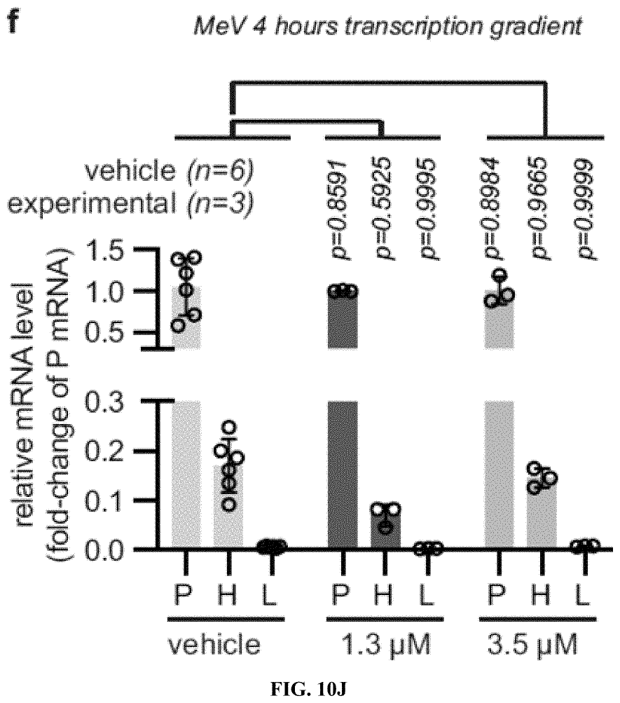

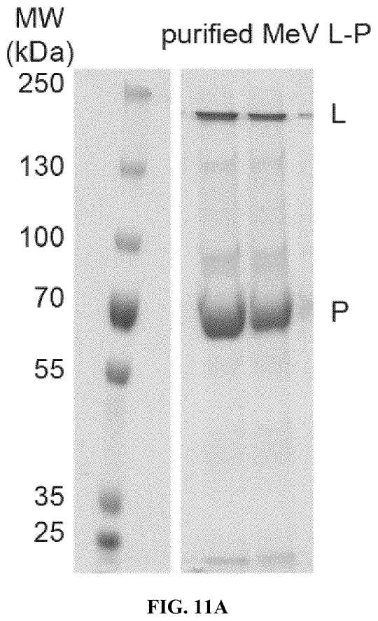

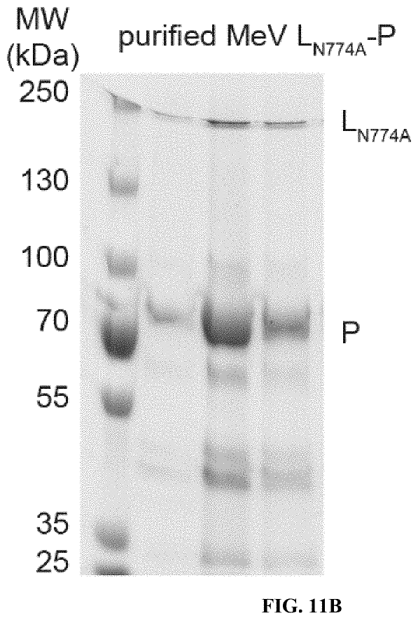

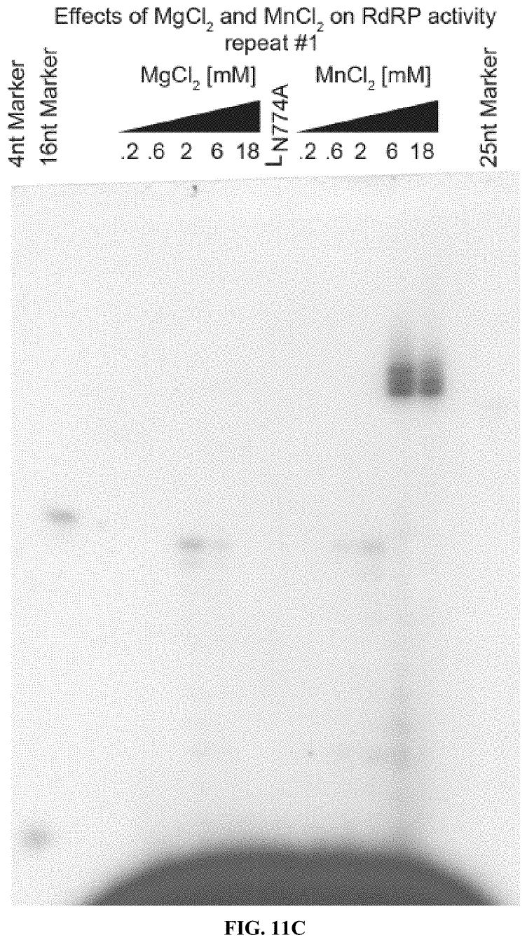

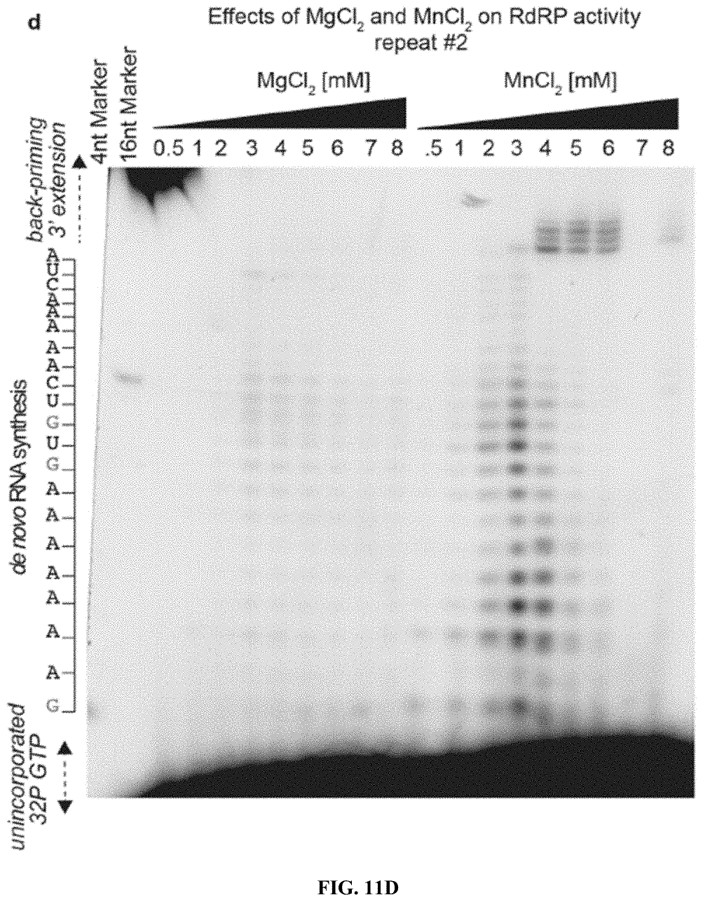

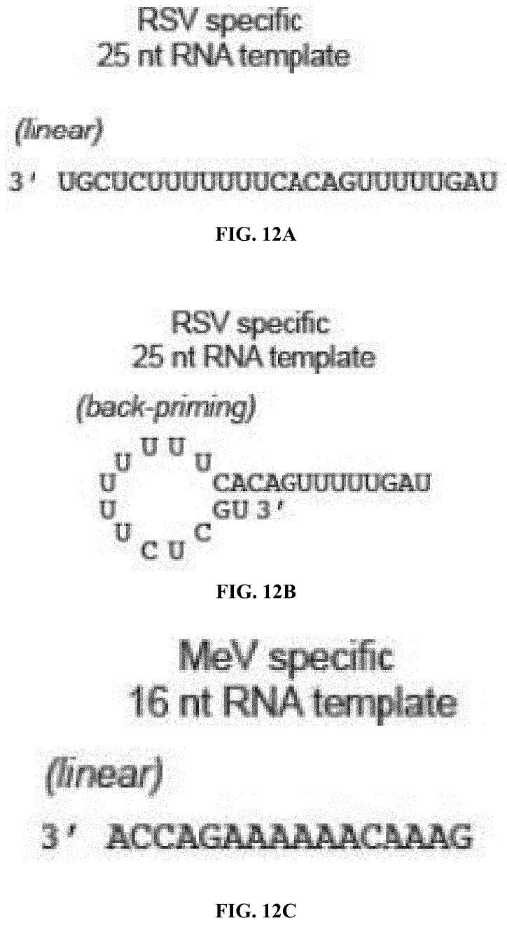

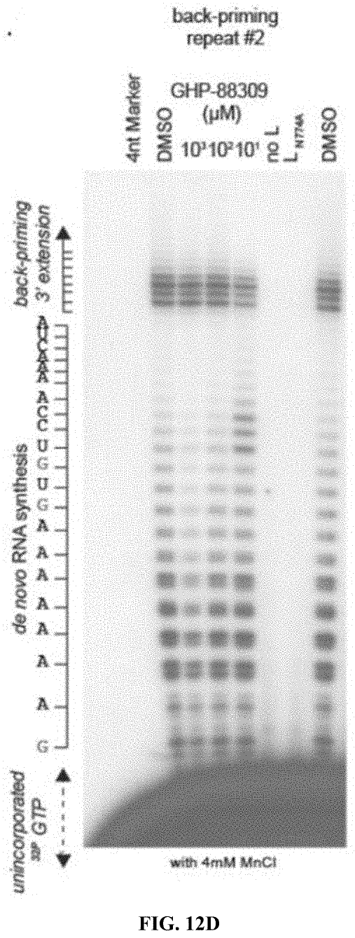

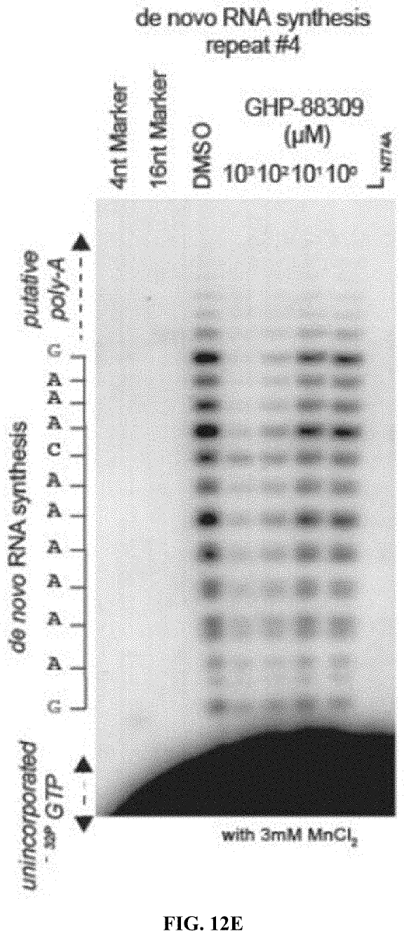

Quantification of relative viral mRNA amounts synthesized in infected cells during primary transcription revealed dose-dependent reduction of both HPIV3 and MeV mRNA by the compound (FIGS. 2H-2J; FIGS. 10E-10G). This outcome could reflect suppressed phosphodiester bond formation, blocked L-mediated mRNA capping, or impaired RdRP initiation at the promoter. We tested aborted mRNA maturation by quantifying the effect of GHP-88309 on HPIV3- or MeV leader (Le)-RNAs, which are the first products of the viral transcriptase after cell entry. The compound dose-dependently inhibited synthesis of Le-RNAs (FIG. 2K; FIG. 10H) but did not alter HPIV3 and MeV mRNA transcription gradients (FIG. 10I-10J). These results are incompatible with impaired mRNA capping since i) mononegavirus Le-RNAs are neither capped nor polyadenylated and should therefore not be affected by blocked capping; ii) RdRPs in transcriptase mode abort when nascent mRNAs remain uncapped⁴⁵, which would result in a steepened viral transcription gradient. We assessed inhibition of RdRP initiation versus RNA elongation in in vitro MeV RdRP assays based on purified recombinant polymerase complexes (FIG. 2L; FIG. 11A-11D; FIG. 12A-E). A 25-mer synthetic RNA template explored effects of GHP-88309 on phosphodiester bond formation and elongation, taking advantage of an artificial back-priming step when the template spontaneously forms a circular hairpin structure (FIG. 2L; FIG. 12A-12B) (Behrens, S. E., et al., EMBO J15, 12-22 (1996), and Noton, S. L., et al., PLoS Pathog. 8, e1002980, (2012)). GHP-88309 did not block RNA elongation after back-priming, and therefore does not prevent phosphodiester bond formation (FIG. 2L; FIG. 12C). However, we noted dose-dependent inhibition of de novo RNA synthesis when using a 16-mer RNA template that does not support back-priming (FIG. 12D-12E), albeit at considerably higher compound concentrations than EC₅₀ values. Testing of a catalytically inactive L_{N774A} mutant confirmed specificity of the in vitro polymerase assay (FIG. 2L; FIG. 12C, 12E).

GHP-88309 is Efficacious in Disease-Relevant 3D Human Airway Organoids

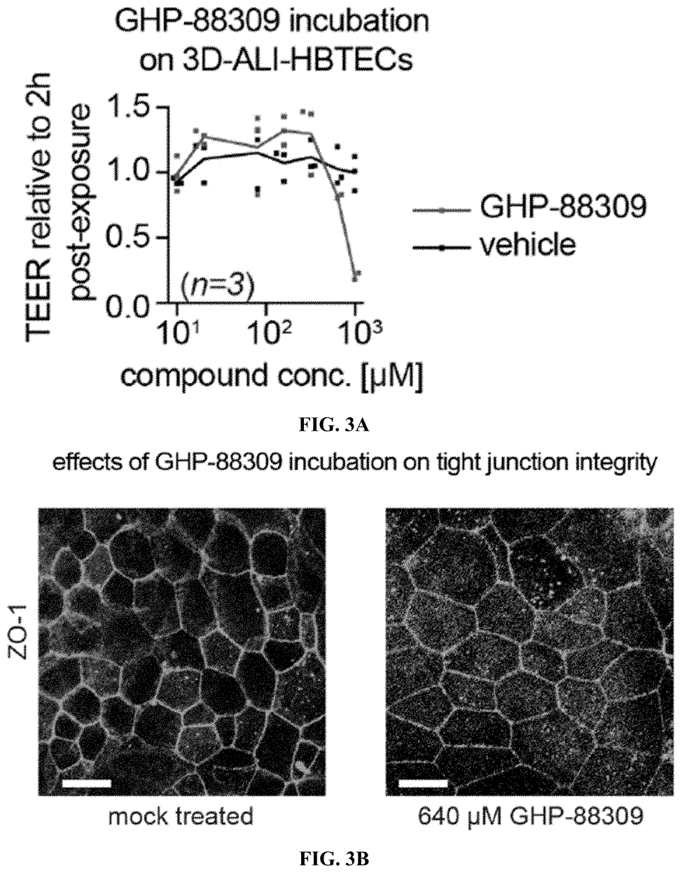

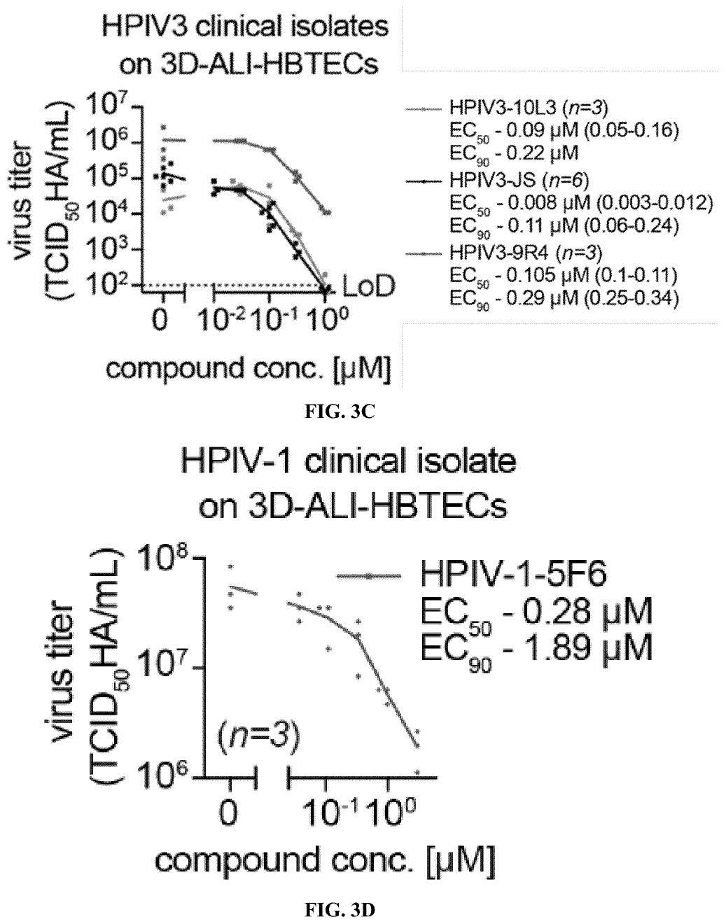

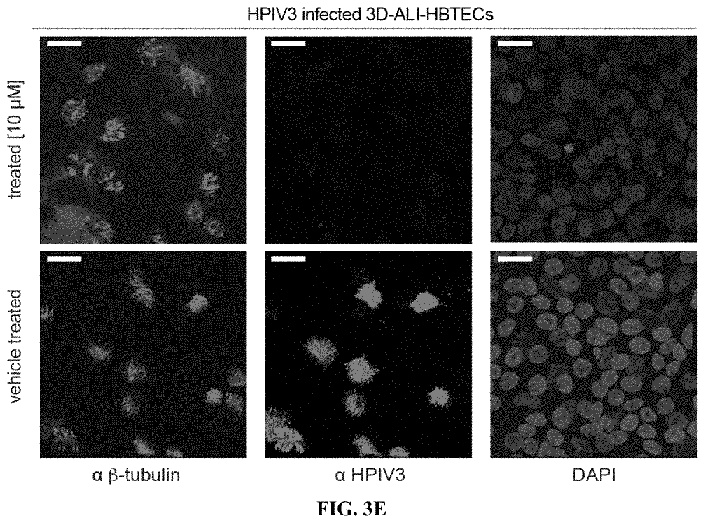

In humans, HPIV3 infection typically remains localized to the respiratory tract. To assess antiviral potency in a disease-relevant human tissue model, we generated well-differentiated primary human bronchial tracheal airway epithelium cultures grown at air-liquid interface (3D-ALI-HBTEC). Transepithelial electrical resistance (TEER), and microscopically-examined tight junction organization were unchanged after organoid exposure to up to 640 μM GHP-88309 in the basolateral chamber (FIG. 3A-B), confirming a wide safety margin.

Dose-response curves generated for HPIV3 isolates 10L3 and 9R4, and HPIV1 isolate 5F6 returned EC₅₀s in the nanomolar range (90 nM, 105 nM, 280 nM, respectively) in 3D-ALI-HBTECs (FIG. 3C-D). Confocal microscopy of infected and treated organoid cultures confirmed virus replication predominantly in ciliated cells, consistent with previous reports, and identified a static GHP-88309 concentration of 10 μM in the basolateral chamber as sterilizing (FIG. 3E).

Oral Bioavailability of GHP-88309

Since established small-animal models of HPIV3 are only semi-permissive and none recapitulates all key features of human disease, we selected the SeV mouse surrogate model to monitor efficacy against respirovirus disease in a natural host. Accordingly, PK profiles were determined in mice.

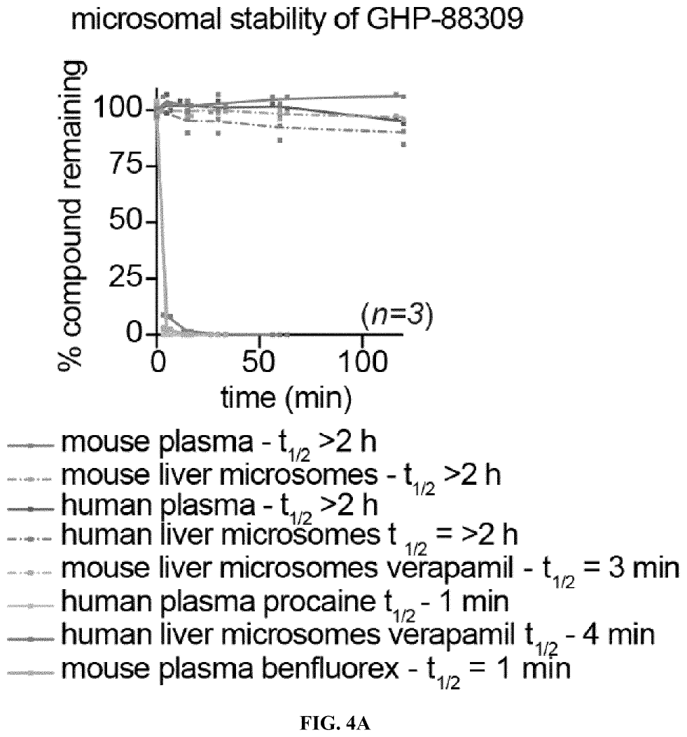

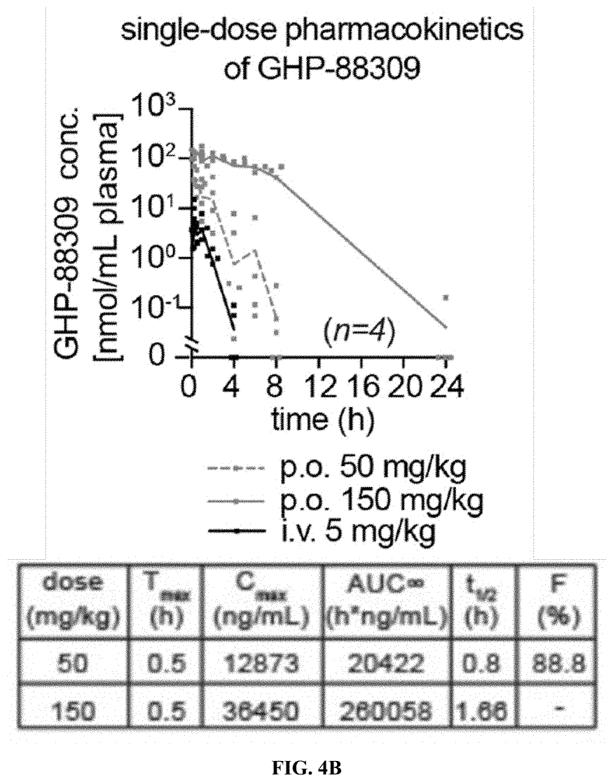

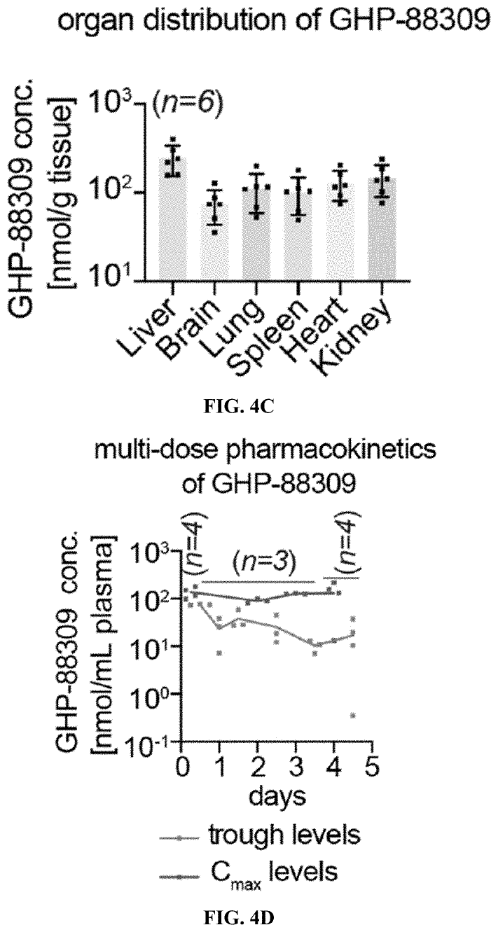

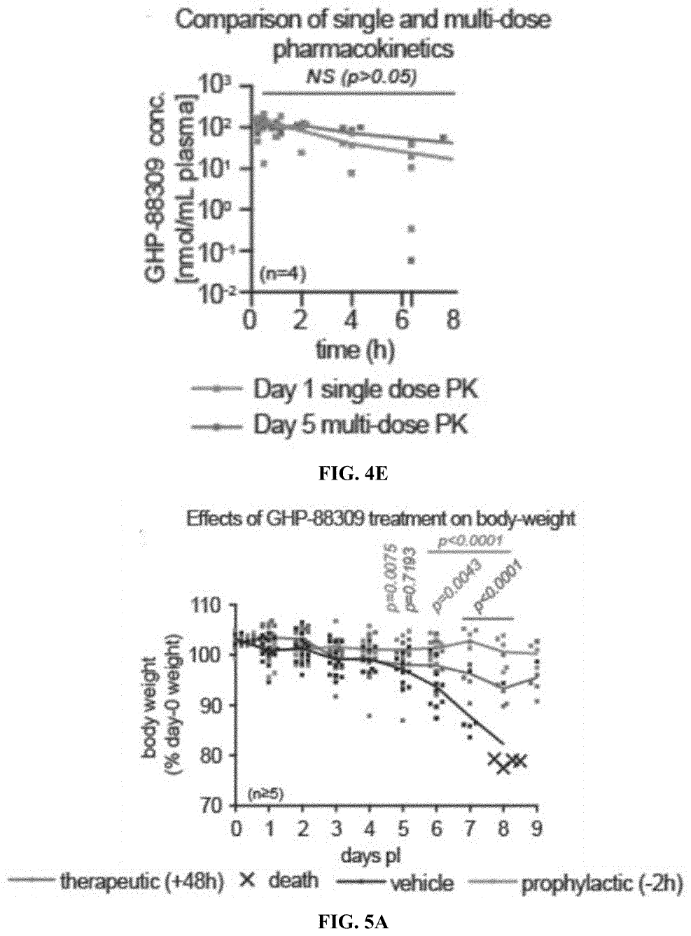

Metabolic stability testing in the presence of human and mouse liver microsomes showed long half-lives of 15.3 and >24 hours, respectively (FIG. 4A). Single-dose PK assays in mice with 50 and 150 mg/kg oral GHP-88309 revealed a dose-dependent, albeit non-linear, overall drug exposure, sustained drug plasma concentrations >30 μM after a 150 mg/kg dose, and oral bioavailability approaching 90% (FIG. 4B; FIG. 21). Drug tissue distribution 60 minutes after plasma Cₘₐₓ corroborated high plasma stability, showing GHP-88309 organ concentrations ranging 70-246 nmol/g in all soft tissues tested, including lung (FIG. 4C). The compound was well tolerated in a 5-day multi-dose oral PK study with b.i.d. administration at 150 mg/kg. Exposed animals developed no signs of biotoxicity despite very high sustained drug plasma concentration that plateaued at ˜34 μM at trough—equivalent to 17-times the cell culture EC₅₀ for SeV—over a 4-day treatment period (FIG. 4D). Multi-day dosing did not significantly (P>0.82) alter PK properties of GHP-88309 (FIG. 4E).

GHP-88309 is Orally Efficacious in HPIV Disease Surrogate Model

SeV in mice recapitulates HPIV pathology, but mice succumb to infection within 8-10 days, establishing a concise therapeutic endpoint. Since cell culture EC %) concentrations of GHP-88309 were ˜6-17-fold lower against HPIV3, HPIV1, and MeV than SeV, the SeV/mouse system promises to be a robust predictor of antiviral efficacy.

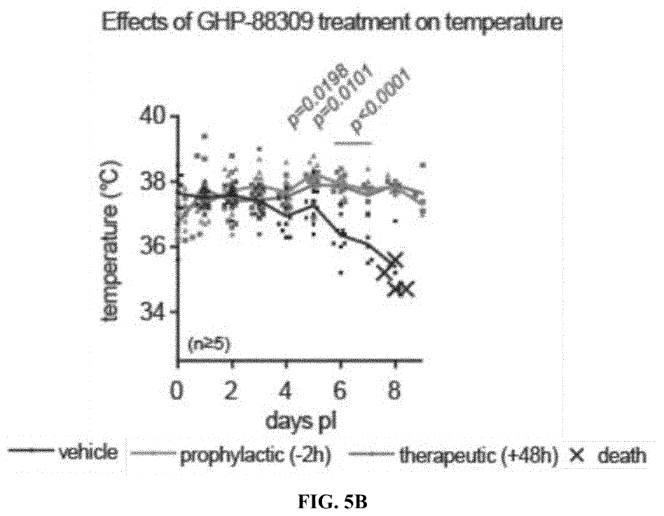

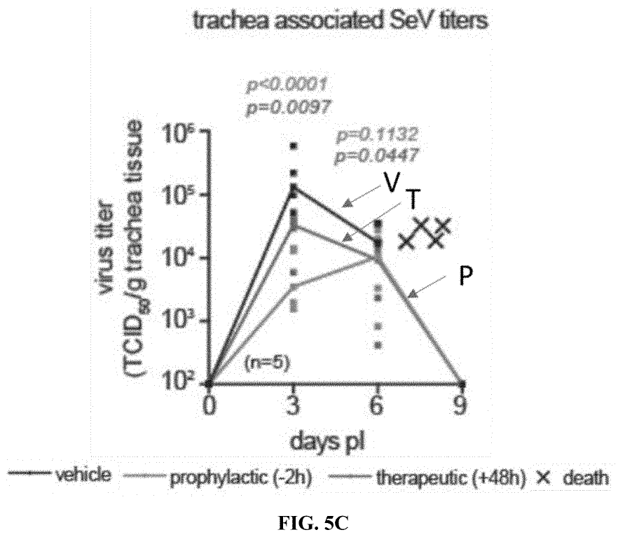

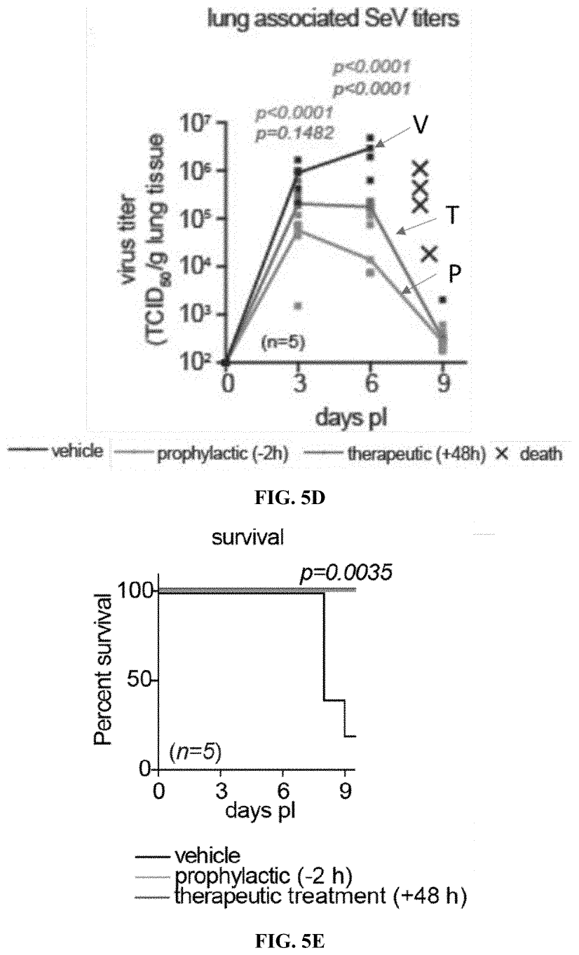

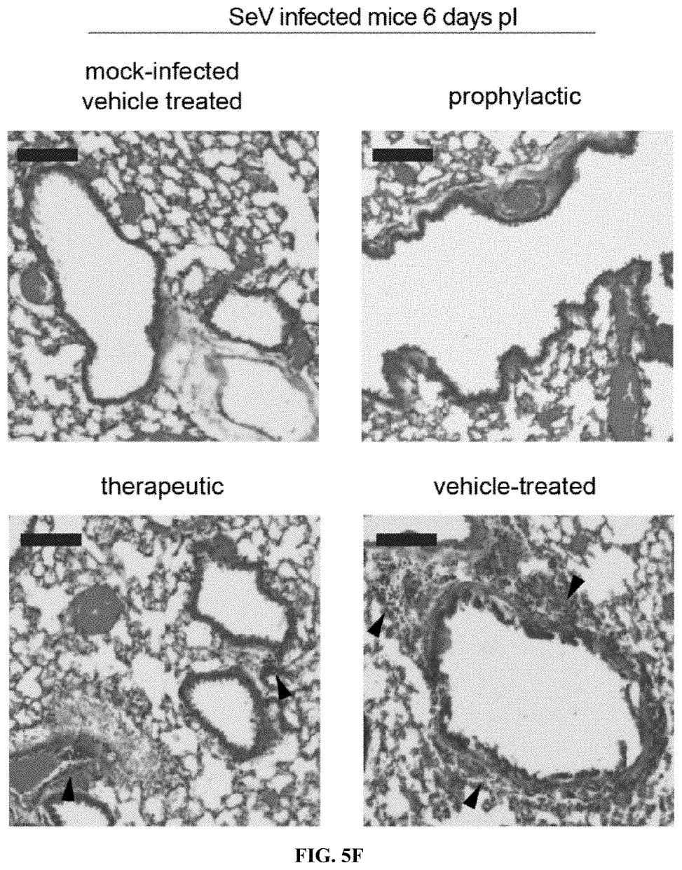

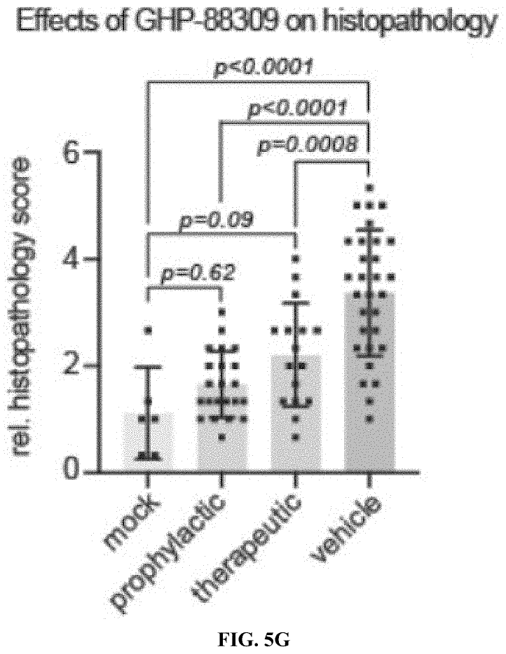

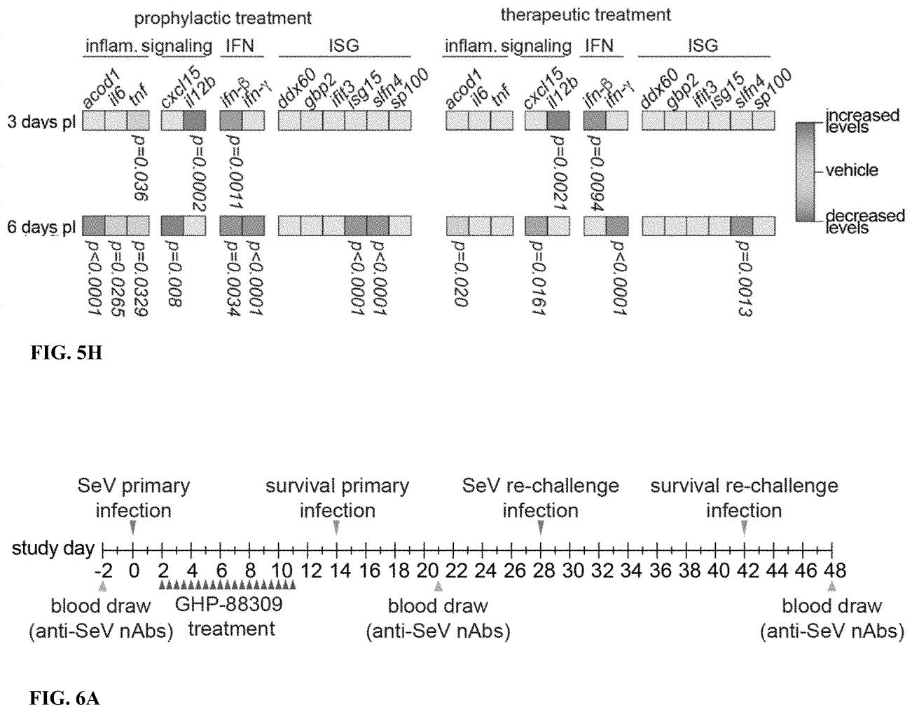

GHP-88309 was administered orally at 150 mg/kg b.i.d., starting at the time of infection (prophylactically) or 48 hours post infection (pI), when lower respiratory tract disease was established (therapeutically). Both regimens alleviated body weight losses and prevented hypothermia (FIG. 5A-B). All treated animals survived, whereas mice of the vehicle-treated group succumbed (predefined endpoint >20% loss of body weight) within 9 days (FIG. 5E). Treatment significantly reduced virus load in trachea (FIG. 5C; P_{therapeutic}=0.0068 and P_{prophylactic}<0.0001) and lungs (FIG. 5D; P<0.0001).

Histological assessment of lung sections six days pI revealed cellular infiltrates in vehicle-treated animals, coinciding with bronchiolar inflammation (FIG. 5F-G). Lung sections of prophylactically treated animals showed no statistically significant (P=0.62) pathological changes. Inflammation and cellular infiltrates were alleviated in lungs of animals treated therapeutically.

Treatment Results in Pharmacological Virus Attenuation

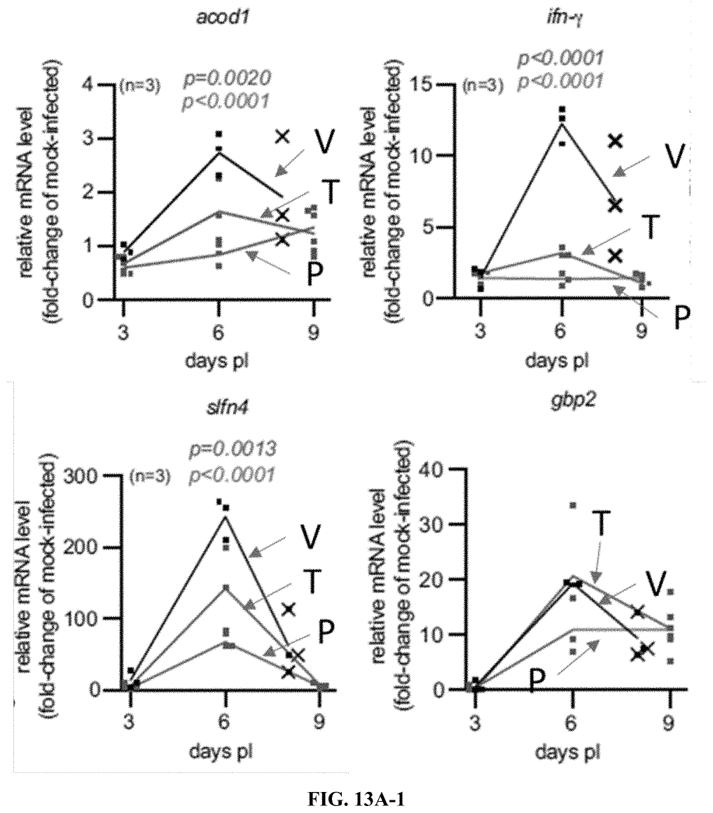

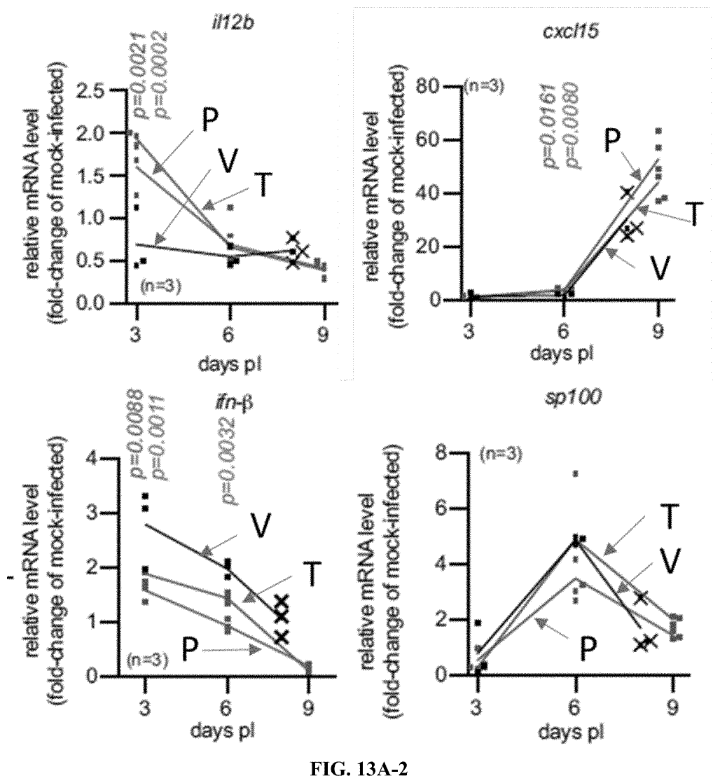

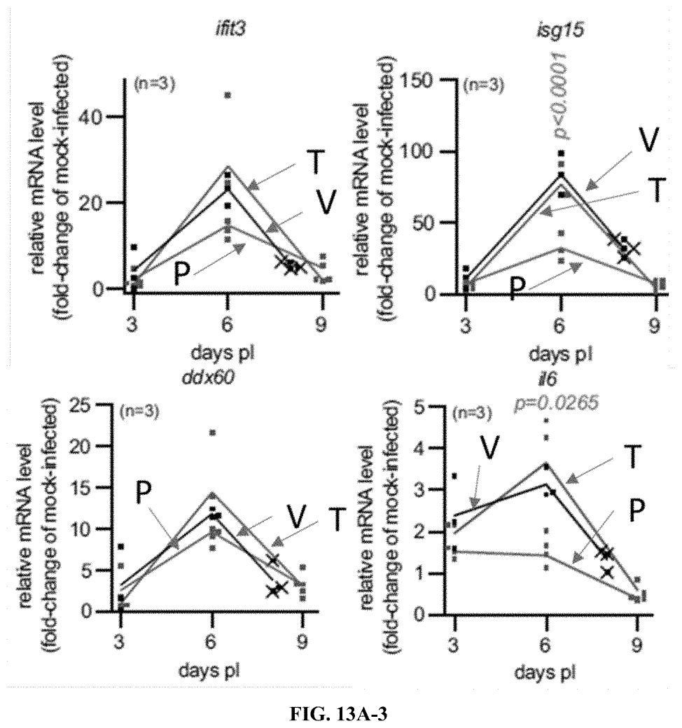

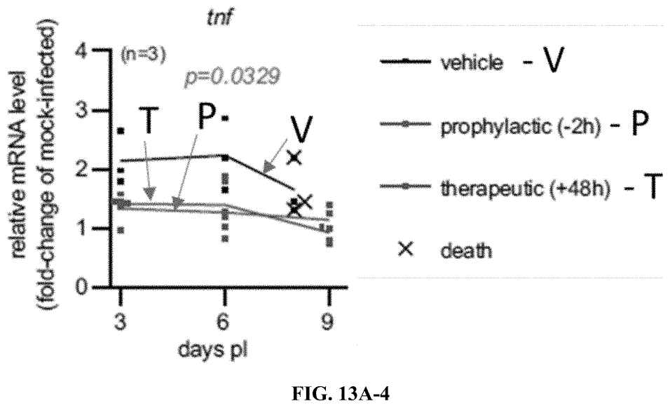

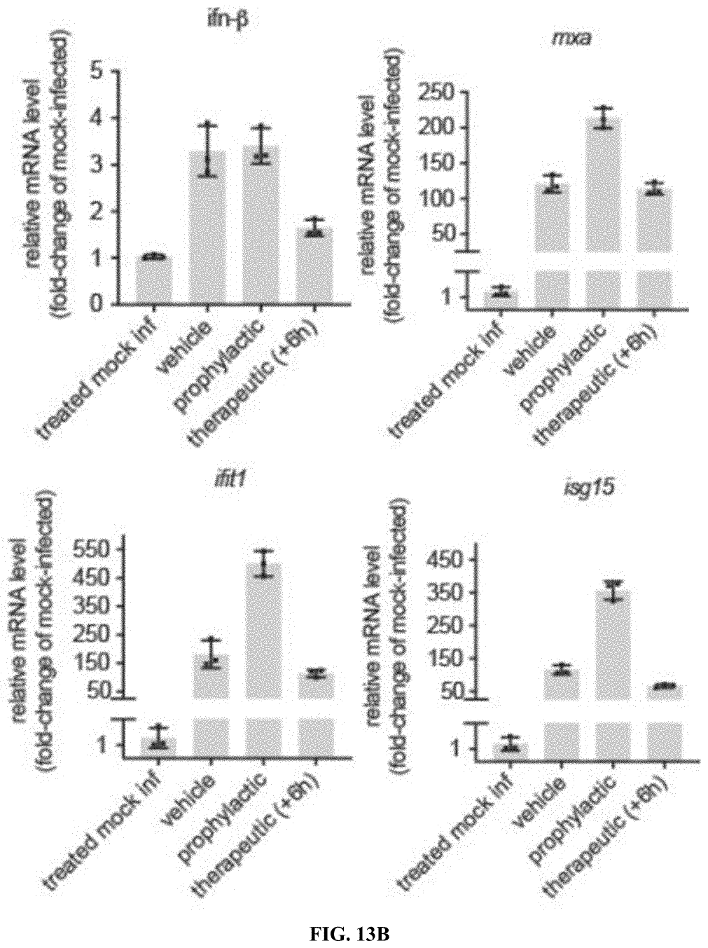

We determined the relative induction of signature genes of the host innate antiviral response in lung tissue three, six, and nine days pI (FIG. 5H; FIG. 13A). IFN-β induction, which is directly correlated with SeV pathogenesis, was significantly lower (P_{therapeutic}=0.0088; P_{prophylactic}=0.0011) in treated mice on day 3 pI than in vehicle animals. Ex vivo treatment of HPIV3-infected HBTECs with GHP-88309 did not reduce IFN-β levels (FIG. 13B), confirming no direct interference with IFN expression.

Paramyxovirus nonstructural proteins, C and V in the case of SeV, counteract host antiviral responses. Mice in treatment groups showed increased IL12b expression on day 3 despite a lower virus burden than in untreated animals, indicating unmitigated host immune activation and thus direct pharmacological virus attenuation by GHP-88309. By day 6, however, treated animals showed significantly decreased expression of inflammatory markers (IL-6 (P_{prophylactic}=0.0265), TNF (P_{prophylactic}=0.0329 and Acod1 (P_{prophylactic}<0.0001, P_{therapeutic}=0.020)) and of some interferon stimulated genes (isg15, slfn4) with antiviral effector function. Infected, treated HBTECs shows enhanced ISG expression (FIG. 13B), consistent with unmitigated IFN-β signaling pathways.

Strong Adaptive Immunity in Recoverees

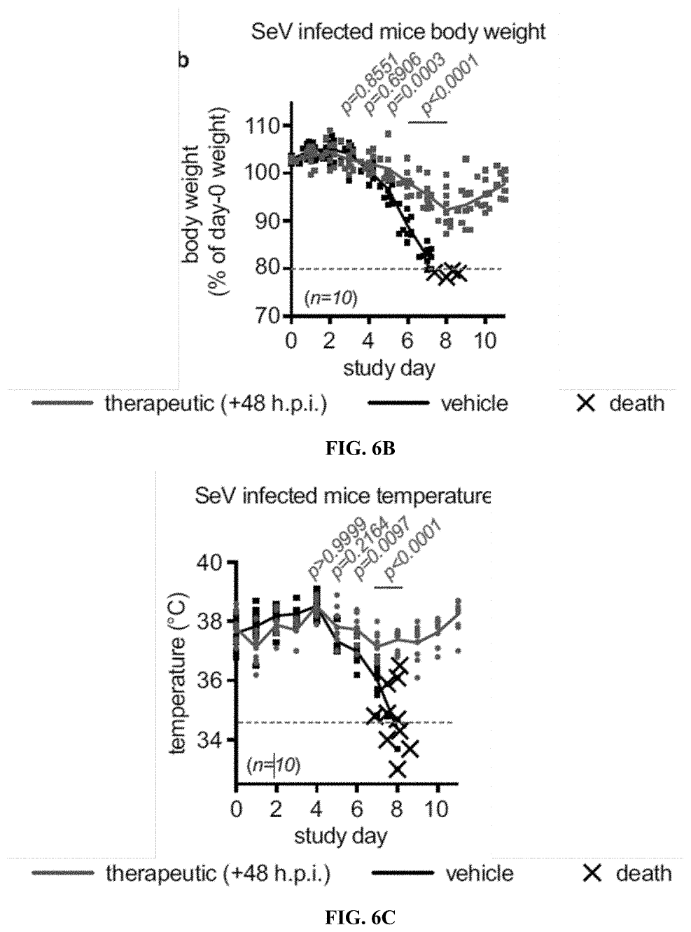

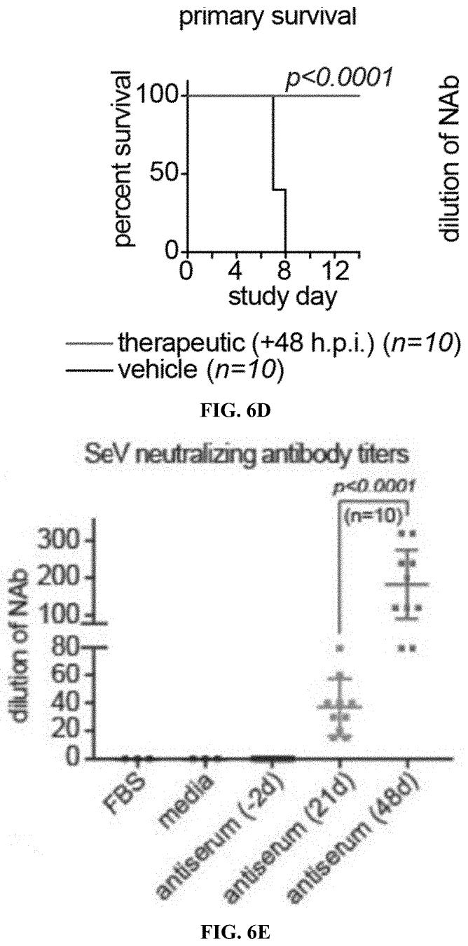

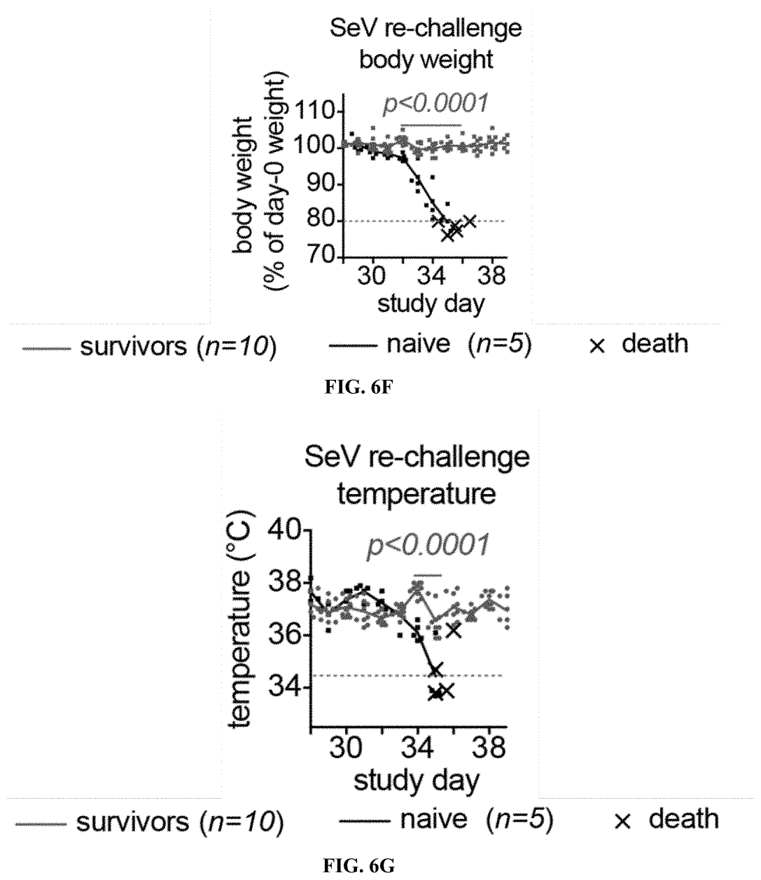

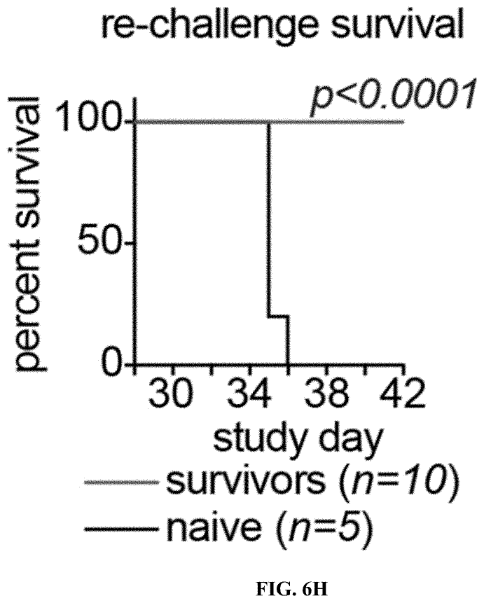

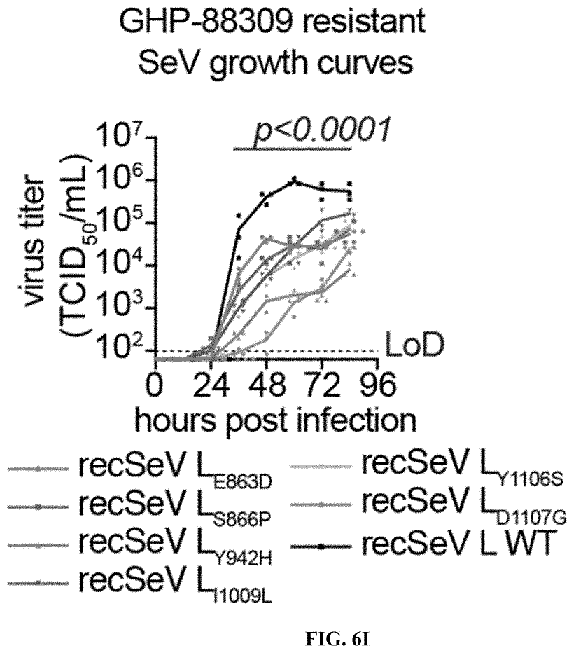

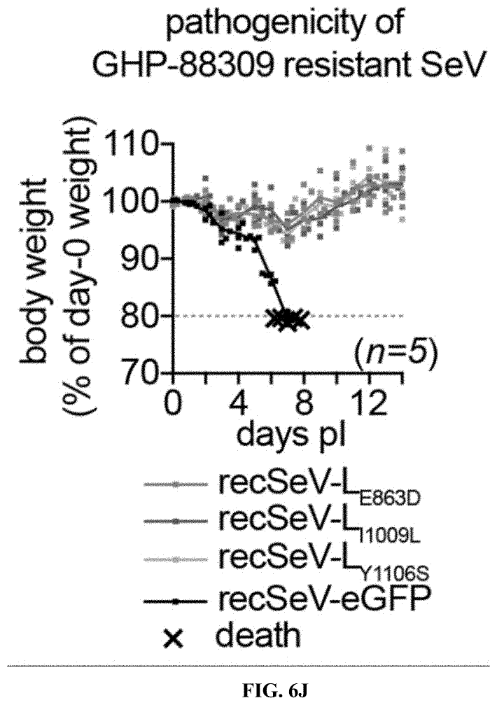

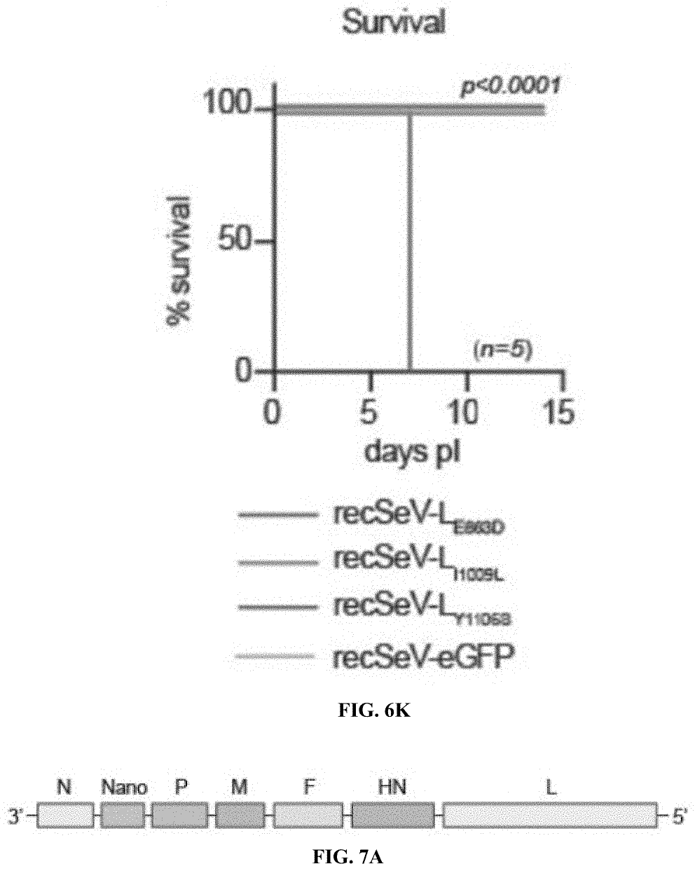

Altered immune responses to paramyxovirus and pneumovirus infection can lead to exacerbated atypical disease upon rechallenge, as described for formalin-inactivated experimental MeV and RSV vaccines. To assess the impact of treatment experience on downstream pathogenesis, we designed a rechallenge study (FIG. 6A). SeV-infected animals were treated therapeutically, starting 48 hours pI, for 9 days b.i.d. Treatment significantly alleviated clinical signs (FIG. 6B-C) and all animals survived, whereas controls succumbed (FIG. 6D). Recoverees mounted a robust humoral anti-SeV response (FIG. 6E). Subsequent rechallenge with 1.5×10⁵ TCID₅₀ SeV 28 days after original infection resulted in complete survival without development of clinical signs (FIG. 6G-H), while a fresh group of naïve animals succumbed.

GHP-88309 Resistant Viruses are Apathogenic

To explore whether resistance to GHP-88309 coincides with infection rebound, we rebuilt all confirmed SeV L resistance mutations in recombinant viruses. In cell culture, these recSeVs showed significant growth delays (P<0.0013) compared to the parent virus (FIG. 6I). Three recSeVs were selected for in vivo testing, based on diversity in hot-spot location, best relative fitness in cell culture, and cross-appearance in HPIV3 (recSeV-L_{E863D}) and MeV (recSeV-L_{I1009L} and recSeV-L_{Y1106S}) adaptations. All animals infected with these viruses survived with only minimal weight loss (FIG. 6J-K). Lung virus load was reduced or below detection level six days pI, when standard recSeV titers peak, and two resistant viruses had reverted to wild-type L (FIG. 22).

To explore whether in vivo virus adaptation identifies additional escape mutations, we passaged recSeV twice in mice treated with sub-inhibitory 50 mg/kg GHP-88309. Of five independent lineages, virus was undetectable in two after the second passage. Viruses isolated from the other three lineages showed unchanged susceptibility to GHP-88309, and Sanger sequencing of L regions associated with resistance did not reveal polymorphisms at known escape sites (FIG. 22). These results indicate that SeV resistance to GHP-88309 coincides with strong viral attenuation in vivo. Although a combination of multiple compensatory mutations may restore fitness of a GHP-88309-resistant virus, a high barrier exists against viral escape without corresponding loss of pathogenicity.

Discussion

Most allosteric small-molecule antivirals show a narrow activity spectrum. For the mononegaviruses, antiviral activity spanning more than one genus has not yet been reported for any direct-acting non-nucleoside inhibitor. The discovery of GHP-88309 has shifted this paradigm. Based on sequence alignments, we predict that all respiroviruses and morbilliviruses are susceptible. Obviously, NiV is not inhibited due to a histidine at L₁₁₅₆, but some members of the henipavirus genus such as Cedar virus or Ghanaian bat virus feature a tyrosine and may be sensitive.

Natural resistance of NiV to the drug raised the question of whether use against respiroviruses and morbilliviruses may trigger resistance mutations that make these viruses more NiV-like, possibly resulting in enhanced disease. Tested in the recSeV-mouse pathogenesis model, all SeV resistance mutations resulted in major viral attenuation, alleviating pathogenesis concerns.

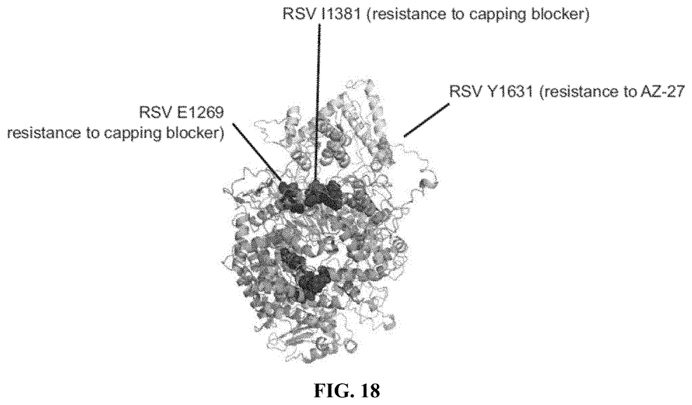

Mechanistic studies consistently pointed at impaired de novo polymerase initiation at the promoter by GHP-88309, reflecting a powerful strategy to block paramyxovirus polymerases. Whereas some inhibitors of related viruses such as RSV-restricted AZ-27 suppress de novo polymerase initiation, resistance profiles and predicted target sites of GHP-88309 and AZ-27 are distinct (FIG. 18). Our top-scoring docking pose posits GHP-88309 at the intersection between the RdRP and capping domains, a prime location to prevent structural rearrangements required for polymerase processivity by bridging both domains. Of note, escape mutations to a series of RSV capping inhibitors map to polymerase regions distinct from GHP-88309 resistance (FIG. 18), underscoring that GHP-88309 does not directly interfere with viral mRNA capping.

PK profiling of GHP-88309 demonstrated that outstanding oral bioavailability and metabolic stability readily compensated for moderate potency deficiencies. Although not dosed to failure yet, even the current orally efficacious 150 mg/kg correspond to a not unrealistic 12 mg/kg human equivalent dose. In contrast to the morbillivirus polymerase inhibitor ERDRP-0519 (Krumm, S A. et al. Sci. Transl. Med. 6, 232-252 (2014)), GHP-88309 did not trigger major increase in ISG expression levels compared to vehicle in the SeV model, which may reflect that SeV infection is limited to respiratory epithelial cells whereas morbilliviruses cause viremia. GHP-88309 provides a unique foundation to dissect the impact of pharmacological intervention with localized and systemic paramyxovirus infections in relevant surrogate models.

Considering the broadened activity spectrum of GHP-88309 and high HPIV disease burden in immunocompromised adults, the compound addresses major concerns that have impaired clinical development of paramyxovirus drugs. Providing a viable path to efficacy assessment, trials could involve adult transplant recipients suffering from devastating HPIV3 infection. In addition to a perceived expanded window of opportunity for therapeutic intervention, frequent monitoring of this group allows for early detection of infection. Our data showcase strong therapeutic potential of the GHP-88309 chemotype, which combines large safety margins with demonstrated oral efficacy, opening opportunities to ultimately address the currently unmet clinical need of patients suffering from respirovirus or morbillivirus disease.

Conclusion

In a high-throughput screen for inhibitors of HPIV3, a major cause of acute respiratory infection, we identified GHP-88309, a non-nucleoside inhibitor of viral polymerase activity that possesses unusual broad-spectrum activity against diverse paramyxoviruses including respiroviruses (i.e. HPIV1 and HPIV3) and morbilliviruses (i.e. MeV). Resistance profiles of distinct target viruses overlap spatially, revealing a conserved binding site in the central cavity of the viral polymerase (L) protein that was validated by photoaffinity labeling-based target mapping. Mechanistic characterization through viral RNA profiling and in vitro MeV polymerase assays identified a block in the initiation phase of the viral polymerase. GHP-88309 showed nanomolar potency against HPIV3 isolates in well-differentiated human airway organoid cultures, was well-tolerated (selectivity index >7,111), orally bioavailable, and provided complete protection against lethal infection in a Sendai virus (SeV)-mouse surrogate model of human HPIV3 disease when administered therapeutically 48 hours after infection. Recoverees had acquired robust immunoprotection against reinfection and viral resistance coincided with severe attenuation. This study provides proof-of-feasibility of a well-behaved broad-spectrum allosteric antiviral and describes a chemotype with high therapeutic potential that addresses major obstacles of anti-paramyxovirus drug development.

Database Searches

Whole genome sequences from RefSeq, GenBank and other NCBI repositories were retrieved using the portal NCBI Virus (https://www.ncbi.nlm.nih.gov/labs/virus/vssi/#/). Searches were performed for complete L protein sequences using the taxonomy ID of HPIV-1 (NCBI:txid12730, 92 sequences), HPIV-3 (NCBI:txid11216, 392 sequences) and MeV (NCBI:txid11234, 293 sequences).

Statistics and Reproducibility

MScreen, GraphPad Prism, and Excel software packages were used for data analysis. One-way or two-way ANOVA with Dunnett's, Sidak's, or Tukey's multiple comparisons post-hoc tests without further adjustments were used to evaluate statistical significance when more than two groups were compared or datasets contained two independent variables, respectively. The specific statistical test applied to individual studies is specified in figure legends. Source Data file (Plemper_SourceData_Statistics) summarizes the test statistic (effect size, degrees of freedom, P values) for every statistical analysis conducted in this study. Appropriateness of samples sizes was determined through Resource Equation and Power analyses as specified in Source Data file (Plemper_SourceData_Statistics). Effect sizes between groups in ANOVAs were calculated as η²=(SS_{effect})/(SSₜₒₜₐₗ) [one-way ANOVA] and ω²=(SS_{effect}−(df_{effect})(MSₑᵣᵣₒᵣ))/MSₑᵣᵣₒᵣ+SSₜₒₜₐₗ [two-way ANOVA]. Survival data were analyzed using a time-to-event log-rank (Mantel-Cox) test. To determine antiviral potency and cytotoxicity, effective concentrations were calculated from dose-response data sets through 4-parameter variable slope regression modeling; values are expressed with 95% confidence intervals (CIs). Biological repeat refers to measurements taken from distinct samples, and results obtained for each individual biological repeat are shown in the figures along with the exact size (n number) of biologically independent samples, animals, or independent experiments. Measure of center (connecting lines and columns) are means throughout, with the exception of FIGS. 5a-e and 6i, which show medians as specified in figure legends. Error bars represent standard deviations (SD) throughout. For all experiments, the statistical significance level a was set to <0.05, exact P values are shown in individual graphs wherever possible.

Ethics Statement

All animal studies were performed following the Guide for the Care and Use of Laboratory Animals. All experiments were approved by the Institutional Animal Care and Use Committee of Georgia State University (protocol AL17019) and conducted in compliance with the Association for the Accreditation of Laboratory Animal Care guidelines, National Institutes of Health regulations, Georgia State University policy, and local, state, and federal laws. Mice were anesthetized with isoflurane and euthanized using carbon dioxide.

Human Subjects

Normal primary human bronchial tracheal epithelial cells (HBTECs) used in this project were sourced from a commercial provider, LifeLine Cell Technology (https://www.lifelinecelltech.com/human-cell-systems/management/). These specimens were obtained by the vendor under informed consent and adhere to the Declaration of Helsinki, The Human Tissue Act (UK), CFR Title 21, and HIPAA regulations as specified in https://www.lifelinecelltech.com/technical-support/ethics/. All regulatory approval lies with the vendor. LifeLine Cell Technology was not involved in study design and had no role, active or advisory, in the project, which includes no involvement in execution of experiments and interpretation of study results. To protect privacy of donors and tissue suppliers, LifeLine Cell Technology does not provide copies of donor records or tissue source agreements. The vendor holds donor consent and legal authorizations that give permission for all research use. These consent and authorization documents do not identify specific types of research testing. If used for research purposes only, the donor consent applies.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

### Embodiments

Embodiment 1: A compound for use in inhibiting RNA polymerase having the structure of Formula (I):

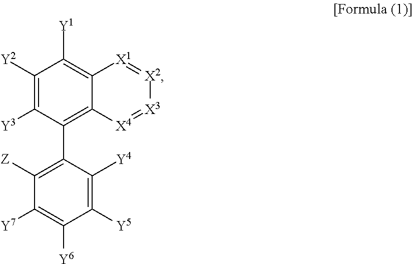

or a pharmaceutically acceptable salt thereof, wherein:
X¹ is selected from CR¹ or N;
X² is selected from CR² or N;
X³ is selected from CR³ or N;
X⁴ is selected from CR⁴ or N;
Z is selected from:

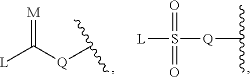

—(CH₂)ₙL, C₁₋₁₀heterocycle, or C₁₋₁₀heteroaryl;
n is from 0-10;
L is selected from R⁵; OR⁵, or NR⁵R⁶;
Y¹ is selected from —F, —Cl, —Br, —I, —NO₂, —CN, -Q¹R^{a}, —N(R^{a})₂, -Q¹C(M)Q¹R^{a}, -Q¹C(O)N(R^{a})₂, -Q¹SO₂Q¹R^{a}, or -Q¹SO₂)N(R^{a})₂,
Y² is selected from —F, —Cl, —Br, —I, —NO₂, —CN, -Q¹R^{b}, —N(R^{b})₂, -Q¹C(M)Q¹R^{b}, -Q¹C(O)N(R^{b})₂, -Q¹SO₂Q¹R^{b}, or -Q¹SO₂N(R^{b})₂,
Y³ is selected from —F, —Cl, —Br, —I, —NO₂, —CN, -Q¹R^{c}, N(R^{c})₂, -Q¹C(M)Q¹R^{c}, -Q¹C(M)N(R^{c})₂, -Q¹SO₂Q¹R^{c}, or -Q¹SO₂N(R^{c})₂,
Y⁴ is selected from —F, —Cl, —Br, —I, —NO₂, —CN, -Q¹R^{d}, —N(R^{d})₂, -Q¹C(M)Q¹R^{d}, -Q¹C(M)N(R^{d})₂, -Q¹SO₂Q¹R^{d}, or -Q¹SO₂N(R^{d})₂,
Y⁵ is selected from —F, —Cl, —Br, —I, —NO₂, —CN, -Q¹R^{e}, —N(R^{e})₂, -Q¹C(M)Q¹R^{e}, -Q¹C(M)N(R^{e})₂, -Q¹SO₂Q¹R^{e}, or -Q¹SO₂N(R^{e})₂,
Y⁶ is selected from —F, —Cl, —Br, —I, —NO₂, —CN, -Q¹R^{f}, —N(R^{f})₂, -Q¹C(M)Q¹R^{f}, -Q¹C(M)N(R^{f})₂, -Q¹SO₂Q¹R^{f}, or -Q¹SO₂N(R^{f})₂,
Y⁷ is selected from —F, —Cl, —Br, —I, —NO₂, —CN, -Q¹R^{g}, —N(R^{g})₂, -Q¹C(M)Q¹R^{g}, -Q¹C(M)N(R^{g})₂, -Q¹SO₂Q¹R^{g}, or -Q¹SO₂N(R^{g})₂,
M is in each case independently selected from O, NH, S, NOH, or CH;
Q is in each case independently selected from null, O, NH, or S;
Q¹ is in each case independently selected from null, O, NH, or S;
R¹, R², R³, and R⁴, are independently selected from R^{p}, OR^{p}, N(R^{p})₂, CN, NO₂, COR^{p}, C(O)OR^{p}, Fl, Cl, Br, or I, wherein R^{p} is in each case independently selected from H, C₁₋₁₀ alkyl, C₁₋₁₀haloalkyl, aryl, C₁₋₁₀heterocycle, or C₁₋₁₀heteroaryl;
R⁵, R⁶, R^{a}, R^{b}, R^{c}, R^{d}, R^{e}, R^{f}, and R^{g} are in each case independently selected from H, C₁₋₁₀alkyl, C₁₋₁₀haloalkyl, aryl, C₁₋₁₀heterocycle, or C₁₋₁₀heteroaryl.
wherein any two or more of L, R¹, R², R³, R⁴, R⁵, R⁶, Y¹, Y², Y³, Y⁴, Y⁵, Y⁶, Y⁷, R^{a}, R^{b}, R^{c}, R^{d}, R^{e}, R^{f}, and R^{g} can together form a ring.
   Embodiment 2: The compound according to any preceding Embodiment, having the structure:

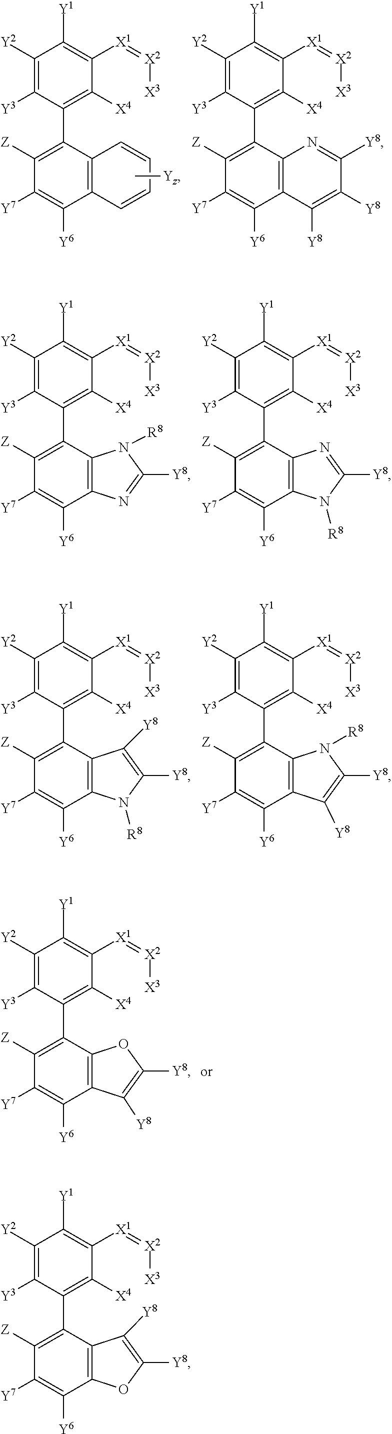

wherein R⁸ is selected from H or C₁₋₃alkyl, and Y⁸ is in each case independently selected from R^{p}, OR^{p}, N(R^{p})₂, CN, NO₂, COR^{p}, C(O)OR^{p}, Fl, Cl, Br, or I, wherein R^{p} is in each case independently selected from H, C₁₋₁₀alkyl, C₁₋₁₀haloalkyl, aryl, C₁₋₁₀heterocycle, or C₁₋₁₀ heteroaryl.

Embodiment 3: The compound of any preceding Embodiment, having the structure:

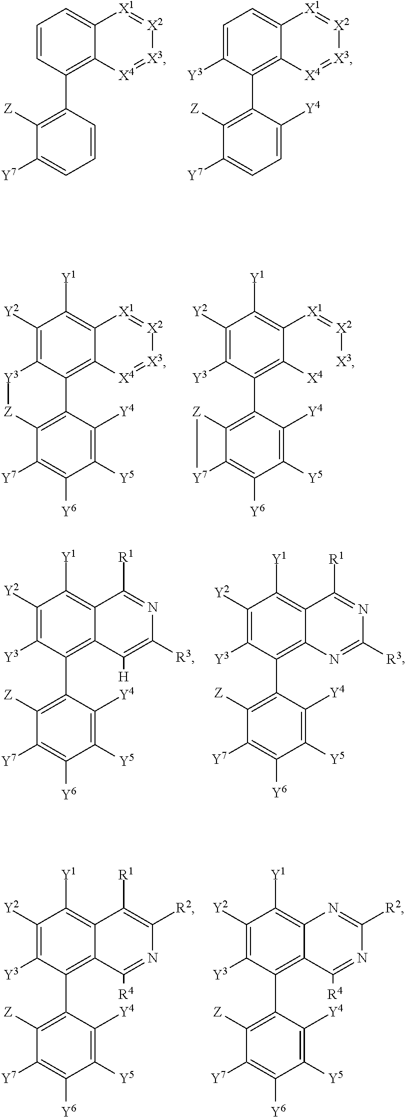

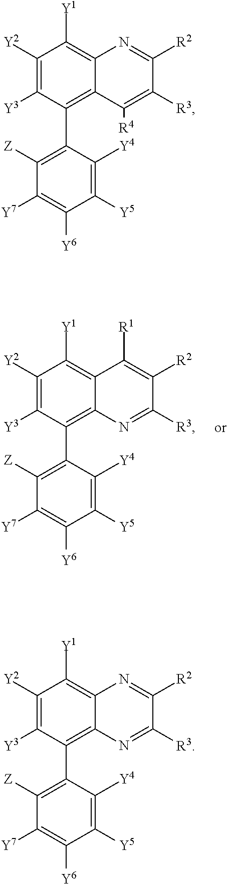

Embodiment 4: The compound of any preceding Embodiment, wherein R¹, R², R³, and R⁴ are independently selected from OH, NH₂, OR^{p} or H; wherein R^{p} is C₁₋₁₀ alkyl.
Embodiment 5: The compound of any preceding Embodiment, wherein Z is a C₁₋₁₀heteroaryl group or a group having the formula:

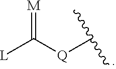

Embodiment 6: The compound of any preceding Embodiment, wherein Q is O or null, preferably null, M is O, and L is OR⁵ or NR⁵R⁶, preferably OH or NH₂.
Embodiment 7: The compound of any preceding Embodiment, wherein Y⁵ is selected from —F, —Cl, —Br, —I, —NO₂, —CN, -Q¹R^{e}, —N(R^{e})₂, -Q¹C(M)Q¹R^{e}, -Q¹C(M)N(R^{e})₂, -Q¹SO₂Q¹R^{e}, or -Q¹SO₂N(R^{e})₂, wherein R^{e} is selected from H, C₁₋₁₀alkyl, C₁₋₁₀haloalkyl, aryl, C₁₋₁₀heterocycle, or C₁₋₁₀heteroaryl.
Embodiment 8: The compound of any preceding Embodiment, wherein Y⁵ is selected from —F, —Cl, or Q¹R^{e1}, wherein Q¹ is null or O, and R^{e1} is C₁₋₄alkyl or C₁₋₄haloalkyl.
Embodiment 9: The compound of any preceding Embodiment, wherein Y⁵ is CF₃, OCF₃, CH₂CF₃, or OCH₂CF₃.
Embodiment 10: The compound of any preceding Embodiment, wherein Y⁷ is selected from —F, —Cl, —Br, —I, —NO₂, —CN, -Q¹R^{g}, —N(R^{g})₂, -Q¹C(M)Q¹R^{g}, -Q¹C(M)N(R^{g})₂, -Q¹SO₂Q¹R^{g}, or -Q¹SO₂N(R^{g})₂, wherein R^{g} is selected from H, C₁₋₁₀alkyl, C₁₋₁₀haloalkyl, aryl, C₁₋₁₀heterocycle, or C₁₋₁₀heteroaryl.
Embodiment 11: The compound of any preceding Embodiment, wherein Y⁷ is selected from —F, —Cl, or -Q¹R^{g1}, wherein Q¹ is null or O, and R^{g1} is C₁₋₄ alkyl or C₁₋₄haloalkyl.
Embodiment 12: The compound of claim, wherein Y⁷ is CF₃, OCF₃, CH₂CF₃, or OCH₂CF₃.
Embodiment 13: The compound of any preceding Embodiment, wherein one or more of Y¹, Y², Y³, Y⁴, and Y⁶ are each hydrogen.
Embodiment 14: The compound of any preceding Embodiment, wherein four of Y¹, Y², Y³, Y⁴, and Y⁶ are hydrogen.
Embodiment 15: The compound of any preceding Embodiment, wherein all of Y¹, Y², Y³, Y⁴, and Y⁶ are each hydrogen.
Embodiment 16: The compound of any preceding Embodiment, wherein Y¹ is selected from —F, —Cl, —Br, —I, —NO₂, —CN, -Q¹R^{a}, —N(R^{a})₂, -Q¹C(M)Q¹R^{a}, -Q¹C(O)N(R^{a})₂, -Q¹SO₂Q¹R^{a}, or -Q¹SO₂N(R^{a})₂, wherein R^{a} is H, C₁₋₁₀alkyl, C₁₋₁₀haloalkyl, aryl, C₁₋₁₀heterocycle, or C₁₋₁₀heteroaryl.
Embodiment 17: The compound of any preceding Embodiment, wherein Y² is selected from —F, —Cl, —Br, —I, —NO₂, —CN, -Q¹R^{b}, —N(R^{b})₂, -Q¹C(M)Q¹R^{b}, -Q¹C(O)N(R^{b})₂, -Q¹SO₂Q¹R^{b}, or -Q¹SO₂N(R^{b})₂, wherein R¹ is H, C₁₋₁₀alkyl, C₁₋₁₀haloalkyl, aryl, C₁₋₁₀heterocycle, or C₁₋₁₀heteroaryl.
Embodiment 18: The compound of any preceding Embodiment, wherein Y³ is selected from —F, —Cl, —Br, —I, —NO₂, —CN, -Q¹R^{c}, N(R^{c})₂, -Q¹C(M)Q¹R^{c}, -Q¹C(M)N(R^{c})₂, -Q¹SO₂Q¹R^{c}, or -Q¹SO₂N(R^{c})₂, wherein R^{c} is H, C₁₋₁₀alkyl, C₁₋₁₀haloalkyl, aryl, C₁₋₁₀heterocycle, or C₁₋₁₀heteroaryl.
Embodiment 19: The compound of any preceding Embodiment, wherein Y⁴ is selected from —F, —Cl, —Br, —I, —NO₂, —CN, -Q¹R^{d}, —N(R^{d})₂, -Q¹C(M)Q¹R^{d}, -Q¹C(M)N(R^{d})₂, -Q¹SO₂Q¹R^{d}, or -Q¹SO₂N(R^{d})₂, wherein R^{d} is H, C₁₋₁₀alkyl, C₁₋₁₀haloalkyl, aryl, C₁₋₁₀heterocycle, or C₁₋₁₀heteroaryl.
Embodiment 20: The compound of any preceding Embodiment, wherein Y⁶ is selected from —F, —Cl, —Br, —I, —NO₂, —CN, -Q¹R^{f}, —N(R^{f})₂, -Q¹C(M)Q¹R^{f}, -Q¹C(M)N(R^{f})₂, -Q¹SO₂Q¹R^{f}, or -Q¹SO₂N(R^{f})₂, wherein R^{f} is H, C₁₋₁₀alkyl, C₁₋₁₀haloalkyl, aryl, C₁₋₁₀heterocycle, or C₁₋₁₀heteroaryl.
Embodiment 21: The compound of any preceding Embodiment, having the structure:

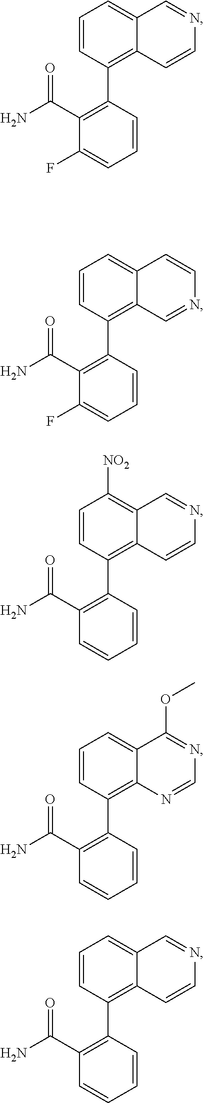

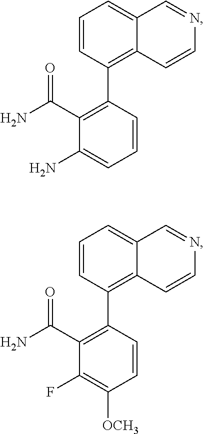 [SMILES: NC(=O)c1c(N)cccc1-c1cccc2cnccc12 ; COc1ccc(-c2cccc3cnccc23)c(C(N)=O)c1F]

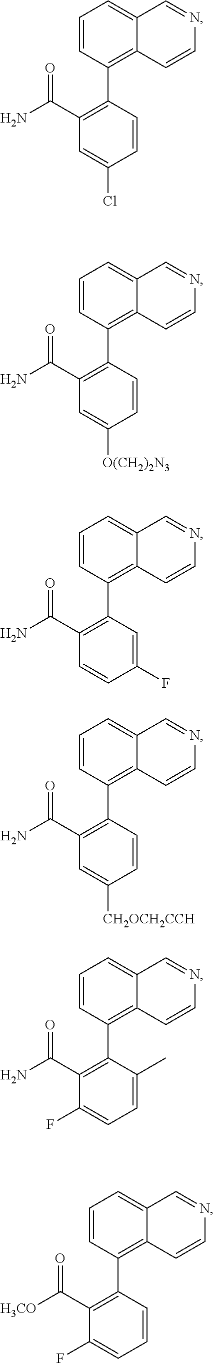

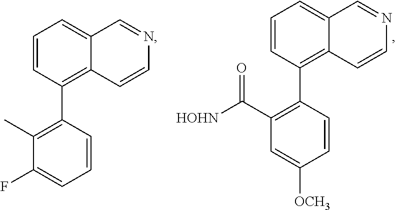

or a pharmaceutically acceptable salt thereof.
Embodiment 22: A method of treating a virus of the Paramyxoviridae family, comprising administering to a patient in need thereof an effective amount of a compound of any preceding claim.
Embodiment 23: The method of Embodiment 22, wherein the virus is measles or human parainfluenza virus.

1. A compound for use in inhibiting RNA polymerase having the structure of Formula (I): 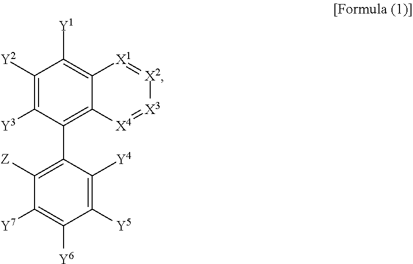
or a pharmaceutically acceptable salt thereof, wherein:
X¹ is selected from CR¹ or N;
X² is selected from CR² or N;
X³ is selected from CR³ or N;
X⁴ is selected from CR⁴ or N;
Z is selected from: 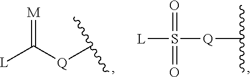
—(CH₂)ₙL, C₁₋₁₀heterocycle, or C₁₋₁₀heteroaryl;
n is from 0-10;
L is selected from R⁵; OR⁵, or NR⁵R⁶;
Y¹ is selected from —F, —Cl, —Br, —I, —NO₂, —CN, -Q¹R^{a}, —N(R^{a})₂, -Q¹C(M)Q¹R^{a}, -Q¹C(O)N(R^{a})₂, -Q¹SO₂Q¹R^{a}, or -Q¹SO₂)N(R^{a})₂,
Y² is selected from —F, —Cl, —Br, —I, —NO₂, —CN, -Q¹R^{b}, —N(R^{b})₂, -Q¹C(M)Q¹R^{b}, -Q¹C(O)N(R^{b})₂, -Q¹SO₂Q¹R^{b}, or -Q¹SO₂N(R^{b})₂,
Y³ is selected from —F, —Cl, —Br, —I, —NO₂, —CN, N(R^{c})₂, -Q¹C(M)Q¹R^{c}, -Q¹C(M)N(R^{c})₂, -Q¹SO₂Q¹R^{c}, or -Q¹SO₂N(R^{c})₂,
Y⁴ is selected from —F, —Cl, —Br, —I, —NO₂, —CN, -Q¹R^{d}, —N(R^{d})₂, -Q¹C(M)Q¹R^{d}, -Q¹C(M)N(R^{d})₂, -Q¹SO₂Q¹R^{d}, or -Q¹SO₂N(R^{d})₂,
Y⁵ is selected from —F, —Cl, —Br, —I, —NO₂, —CN, -Q¹R^{e}, —N(R^{e})₂, -Q¹C(M)Q¹R^{e}, -Q¹C(M)N(R^{e})₂, -Q¹SO₂Q¹R^{e}, or -Q¹SO₂N(R^{e})₂,
Y⁶ is selected from —F, —Cl, —Br, —I, —NO₂, —CN, -Q¹R^{f}, —N(R^{f})₂, -Q¹C(M)Q¹R^{f}, -Q¹C(M)N(R^{f})₂, -Q¹SO₂Q¹R^{f}, or -Q¹SO₂N(R^{f})₂,
Y⁷ is selected from —F, —Cl, —Br, —I, —NO₂, —CN, -Q¹R^{g}, —N(R^{g})₂, -Q¹C(M)Q¹R^{g}, -Q¹C(M)N(R^{g})₂, -Q¹SO₂Q¹R^{g}, or -Q¹SO₂N(R^{g})₂,
M is in each case independently selected from O, NH, S, NOH, or CH;
Q is in each case independently selected from null, O, NH, or S;
Q¹ is in each case independently selected from null, O, NH, or S;
R¹, R², R³, and R⁴, are independently selected from R^{p}, OR^{p}, N(R^{p})₂, CN, NO₂, COR^{p}, C(O)OR^{p}, Fl, Cl, Br, or I, wherein R^{p} is in each case independently selected from H, C₁₋₁₀ alkyl, C₁₋₁₀haloalkyl, aryl, C₁₋₁₀heterocycle, or C₁₋₁₀heteroaryl;
R⁵, R⁶, R^{a}, R^{b}, R^{c}, R^{d}, R^{e}, R^{f} and R^{g} are in each case independently selected from H, C₁₋₁₀haloalkyl, aryl, C₁₋₁₀heterocycle, or C₁₋₁₀heteroaryl.
wherein any two or more of L, R¹, R², R³, R⁴, R⁵, R⁶, Y¹, Y², Y³, Y⁴, Y⁵, Y⁶, Y⁷, R^{a}, R^{b}, R^{c}, R^{d}, R^{e}, R^{f}, and R^{g} can together form a ring.

2. The compound of claim 1, wherein R¹, R², R³, and R⁴ are independently selected from OH, NH₂, OR^{p} or H; wherein R^{p} is C₁₋₁₀ alkyl.

3. The compound of claim 1, wherein Z is a C₁₋₁₀heteroaryl group or a group having the formula:

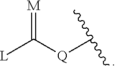

4. The compound of claim 1, wherein Q is O or null, preferably null, M is O, and L is OR⁵ or NR⁵R⁶.

5. The compound of claim 1, wherein Y⁷ is selected from —F, —Cl, —Br, —I, —NO₂, —CN, -Q¹R^{g}, —N(R^{g})₂, -Q¹C(M)Q¹R^{g}, -Q¹C(M)N(R^{g})₂, -Q¹SO₂Q¹R^{g}, or -Q¹SO₂N(R^{g})₂, wherein R^{g} is selected from H, C₁₋₁₀alkyl, C₁₋₁₀haloalkyl, aryl, C₁₋₁₀ heterocycle, or C₁₋₁₀heteroaryl.

6. The compound of claim 1, wherein Y⁷ is CF₃, OCF₃, CH₂CF₃, or OCH₂CF₃.

7. The compound of claim 1, wherein one or more of Y¹, Y², Y³, Y⁴ and Y⁶ are each hydrogen.

8. The compound of claim 1, wherein four of Y¹, Y², Y³, Y⁴, and Y⁶ are hydrogen.

9. The compound of claim 1, wherein all of Y¹, Y², Y³, Y⁴, and Y⁶ are each hydrogen.

10. The compound of claim 1, wherein Y¹ is selected from —F, —Cl, —Br, —I, —NO₂, —CN, -Q¹R^{a}, —N(R^{a})₂, -Q¹C(M)Q¹R^{a}, -Q¹C(O)N(R^{a})₂, -Q¹SO₂R^{a}, or -Q¹SO₂N(R^{a})₂, wherein R^{a} is H, C₁₋₁₀alkyl, C₁₋₁₀haloalkyl, aryl, C₁₋₁₀heterocycle, or C₁₋₁₀heteroaryl.

11. The compound of claim 1, wherein Y⁴ is selected from —F, —Cl, —Br, —I, —NO₂, —CN, -Q¹R^{d}, —N(R^{d})₂, -Q¹C(M)Q¹R^{d}, -Q¹C(M)N(R^{d})₂, -Q¹SO₂Q¹R^{d}, or -Q¹SO₂N(R^{d})₂, wherein R^{d} is H, C₁₋₁₀alkyl, C₁₋₁₀haloalkyl, aryl, C₁₋₁₀heterocycle, or C₁₋₁₀heteroaryl.

12. The compound of claim 1, wherein Y⁶ is selected from —F, —Cl, —Br, —I, —NO₂, —CN, -Q¹R^{f}, —N(R^{f})₂, -Q¹C(M)Q¹R^{f}, -Q¹C(M)N(R^{f})₂, -Q¹SO₂Q¹R^{f}, or -Q¹SO₂N(R^{f})₂, wherein R^{f} is H, C₁₋₁₀alkyl, C₁₋₁₀haloalkyl, aryl, C₁₋₁₀heterocycle, or C₁₋₁₀heteroaryl.

13. The compound of claim 1, having the structure: 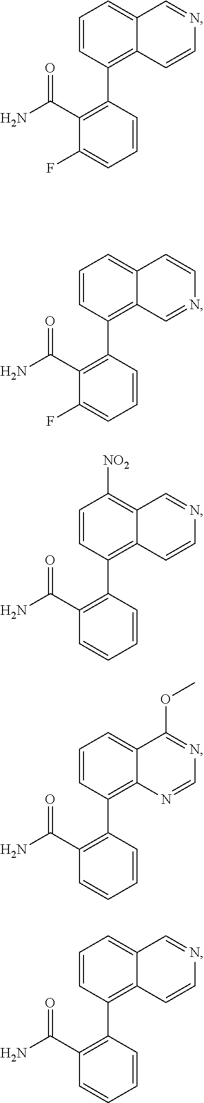 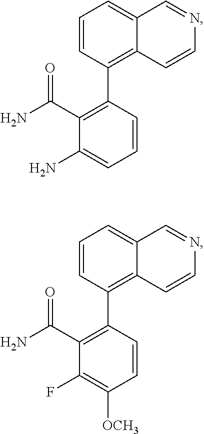 [SMILES: NC(=O)c1c(N)cccc1-c1cccc2cnccc12 ; COc1ccc(-c2cccc3cnccc23)c(C(N)=O)c1F] 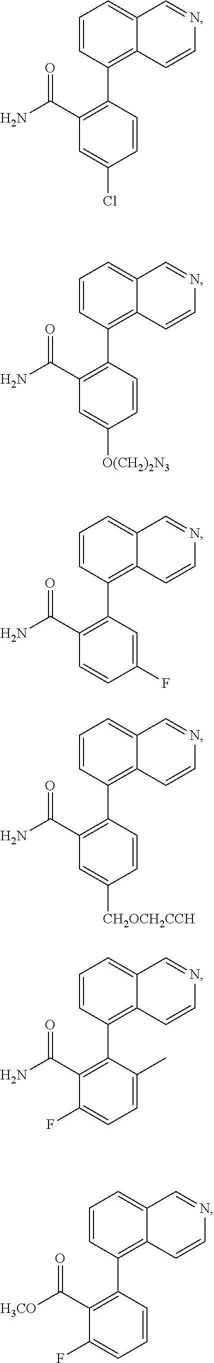 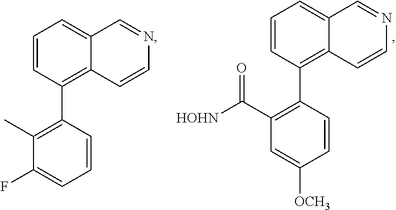
or a pharmaceutically acceptable salt thereof.

14. A method of treating a virus of the Paramyxoviridae family, comprising administering to a patient in need thereof an effective amount of a compound of claim 1.

15. The method of claim 14, wherein the virus is measles or human parainfluenza virus.


SEQUENCE LISTING (118 sequences)

SEQ ID NO: 1 - RNA, 12 nt, ARTIFICIAL SEQUENCE
  Synthetic Sequence
gaaaaaacaaag

SEQ ID NO: 2 - RNA, 21 nt, ARTIFICIAL SEQUENCE
  SYNTHETIC SEQUENCE
gaaaaaaaguguccaaaacua

SEQ ID NO: 3 - RNA, 25 nt, ARTIFICIAL SEQUENCE
  Synthetic Sequence
ugcucuuuuuuucacaguuuuugau

SEQ ID NO: 4 - RNA, 16 nt, ARTIFICIAL SEQUENCE
  Synthetic Sequence
accagaaaaaacaaag

SEQ ID NO: 5 - protein, 120 aa, HUMAN RESPIROVIRUS 3
IYYDGRILPQALKALSRCVFWSETVIDETRSASSNLATSFAKAIENGYSPVLGYACSIFKNIQQLYIALGMNINPTITQNIRDQYFRNPNWMQYASLIPASVGGFNYMAMSRCFVRNIGD

SEQ ID NO: 6 - protein, 120 aa, BOVINE RESPIROVIRUS 3
IYYDGRILPQALKALSRCVFWSETIIDETRSASSNLATSFAKAIENGYSPVLGYVCSIFKNIQQLYIALGMNINPTITQNIRDQYFRNIHWMQYASLIPASVGGFNYMAMSRCFVRNIGD

SEQ ID NO: 7 - protein, 120 aa, HUMAN RESPIROVIRUS 1
IYYDGKILPQCLKALTRCVFWSETLVDENRSACSNIATSIAKAIENGYSPILGYCIALFKTCQQVCISLGMTINPTITSTIRDQYFKGKNWLRCAILIPANIGGFNYMSTARCFVRNIGD

SEQ ID NO: 8 - protein, 120 aa, PORCINE RESPIROVIRUS 1
IYYDGKILPQCLKALTRCVFWSETLVDENRSACSNLATAIAKAIENGYSPILGYSIALYKTCQQVCISLGMTINPTITPSIRDQYYLGKNWLRCAVLIPANVGGFNYMAMSRCFVRNIGD

SEQ ID NO: 9 - protein, 120 aa, MURINE RESPIROVIRUS
IYYDGKILPQCLKALTKCVFWSETLVDENRSACSNISTSIAKAIENGYSPILGYCIALYKTCQQVCISLGMTINPTISPTVRDQYFKGKNWLRCAVLIPANVGGFNYMSTSRCFVRNIGD

SEQ ID NO: 10 - protein, 120 aa, CANINE MORBILLIVIRUS
IYYDGMLISQSLKSIARCVFWSETIVDETRAACSNISTTLAKAIEKGFDRYLAYALNILKIIQQVLISLGFTINSAMTRDVIEPLLQDHCLLTKMAILPAPIGGLNYLNMSRLFVRNIGD

SEQ ID NO: 11 - protein, 120 aa, DOLPHIN MORBILLIVIRUS
IYYDGMLISQSLKSVARCVFWSETIVDETRAACSNISTTLAKSIERGFDRYLAYSLNVLKIIQQILISLGFTINTSMTQDIAIPLLQNQDLLIKMALLPAPIGGLNYLNMSRLFVRNIGD

SEQ ID NO: 12 - protein, 120 aa, MEASLES MORBILLIVIRUS
IYYDGLLVSQSLKSIARCVFWSETIVDETRAACSNIATTMAKSIERGYDRYLAYSLNVLKVIQQILISLGFTINSTMTRDVVIPLLTNNDLLIRMALLPAPIGGMNYLNMSRLFVRNIGD

SEQ ID NO: 13 - protein, 120 aa, PDPR MORBILLIVIRUS
IYYDGLLVSQSLKSIARCVFWSETIVDETRAACSNIATTVAKSIERGYDRYLAYSLNILKIFQQILISLGFTINTTMTQDVVAPIIENGDLLIRMALLPAPIGGLNYLNMSRLFVRNIGD

SEQ ID NO: 14 - protein, 120 aa, PHOCINE MORBILLIVIRUS
IYYDGMLISQSLKSVARCVFWSETIVDETRAACSNISTTLAKAIEKGFDRYLAYALNILKIIQQVLISLGFTINSAMTQDVIEPLLQNHCLLTKMAILPAPIGGLNYLNMSRLFVRNIGD

SEQ ID NO: 15 - protein, 120 aa, RINDERPEST MORBILLIVIRUS
IYYDGLLVSQSLKSIARCVFWSETIVDETRAACSNIATTIAKSIERGFDRYLAYSLNILKIIQQILISLGFTINSTMTQDVVVPLLSNNDLLIRMALLPAPIGGMNYLNMSRLFVRNIGD

SEQ ID NO: 16 - protein, 120 aa, CEDAR VIRUS
IHYDGAVLSQSLKSMARCVFWSETLVDETRAACSNISTTIAKAIENGYSRRSGYLINVLKTIQQINISLSFNINECMTDDIIRPFRDNPNWIKHAALIPASLGGLNYMNMSRLYVRNIGD

SEQ ID NO: 17 - protein, 120 aa, GHANAIAN BAT
IYYDGVMLSQGLKTISRCVFWSETLVDETRSACSNISTAITKSIENGIDHRFGFCLNYLKTMQQLSISLNFSINETITDDIKMAFTNHPEWYKISSMIPAALGGFNYLNVSRLYVRNIGD

SEQ ID NO: 18 - protein, 120 aa, HENDRA HENIPAVIRUS
IHYDGAVLSQALKSMSRCCFWSETLVDETRSACSNISTTIAKAIENGLSRNVGYCINVLKVIQQLLISTEFSINETLTADVTSPISNNLDWLVTASLIPAPIGGFNYLNLSRIFVRNIGD

SEQ ID NO: 19 - protein, 120 aa, MOJIANG VIRUS
IYYDGMVLSQGLKSVSRCVFWSETLVDETRSACSNISTSIAKAIENGLSKDIGYSLNVLKTIQQIIVSLKFSINETMTPDVTNPLIQNINWLLTACLMPASLGGFNYLNLSRLYVRNIGD

SEQ ID NO: 20 - protein, 120 aa, NIPAH HENIPAVIRUS
IHYDGAVLSQALKSMSRCCFWSETLVDETRSACSNISTTIAKAIENGLSRNVGYCINILKVIQQLLISTEFSINETLTLDVTSPISNNLDWLITAALIPAPIGGFNYLNLSRIFVRNIGD

SEQ ID NO: 21 - protein, 120 aa, REPTILIAN FERLAVIRUS
IYFDGSILPQGLKTISRCVFWSETLVDETRAACSNISTSLAKAVENGISPLLCYLLNAWKTLQQLHISLAFSINPTITKDLYGPILSSQDWMIIAVIVPSQLGGFNYMSLSRLFVRNIGD

SEQ ID NO: 22 - protein, 120 aa, AVIAN AVULAVIRUS 1
IFKDGAILSQVLKNSSKLVLISGDLSENTVMSCANIASTIARLCENGLPKDFCYYLNYLMSCVQTYFDSEFSITHSSQSDSNQSWIEDISFVHSYVLTPAQLGGLSNLQYSRLYTRNIGD

SEQ ID NO: 23 - protein, 120 aa, AVIAN AVULAVIRUS 2
IFWEGRILSQALKNATKLCFIADHLGDNTVSSCSNLASTITRLVENGYEKDTAFILNIISAMTQLLIDEQYSLQGDYSAVRKLIGSSNYRNLLVASLIPGQVGGYNFLNISRLFTRNIGD

SEQ ID NO: 24 - protein, 118 aa, AVIAN AVULAVIRUS 3
IFYDGRILPQLLKNISKLTLSATTTGENCLTSCGDLSSCITRCIENGFPKDAAFILNQLTIRTQILADHFYSILGGCFTGLNQHDIRLLLSDGSILPAQLGGFNNLNISRLFCRNIGD

SEQ ID NO: 25 - protein, 118 aa, AVIAN AVULAVIRUS 4
IFFNGSILSQCLKNFSKLTTNATTLAENTVAGCSDISSCIARCVENGLPKDAAYIQNIVMTRLQLLLDHYYSMHGGINSELEQPTLSISVRNATYLPSQLGGYNHLNMTRLFCRNIGD

SEQ ID NO: 26 - protein, 118 aa, AVIAN AVULAVIRUS 5
IFFEGRILSQGLKNAAKLVMISDTTGENTVSNCSNIGSTVARLIENGANKASCWIINWILNMKQIIFDTFFSLSNVSLKHTNLILDPKFLVTISLVPGQLGGLNFLNVSRMFTRNIGD

SEQ ID NO: 27 - protein, 120 aa, AVIAN AVULAVIRUS 6
IFFEGKILSQLLKNASRLVLVSETVGENCVGNCSNISSTVARLIENGLDKRVAWGLNILMIVKQILFDIDFSLEPEPSQGLIHAIRQDPNNIKNISITPAQLGGLNFLALSRLFTRNIGD

SEQ ID NO: 28 - protein, 120 aa, AVIAN AVULAVIRUS 7
VFFEGRVLSQILKNASKLNLISDCLAENTVASCSNISSTVARLIENGLGKDVAFILNFQTIIRQLIFDEVYTISLNYSTARRQVGSENPHALAIAALIPGQLGGFNFLNVARLFTRNIGD

SEQ ID NO: 29 - protein, 120 aa, AVIAN AVULAVIRUS 8
IFFEGRLLSQVLKNAAKLCMVADHLGENTVSSCSNLSSTIARLVENGFEKDTAFVLNLVYIMTQILFDEHYSIVCDHNSVKSLIGSKNYRNLLYSSLIPGQLGGFNFLNISRLFTRNIGD

SEQ ID NO: 30 - protein, 120 aa, AVIAN AVULAVIRUS 9
VFKDGKILSQALKNAAKLVLISGEIGENTPMSCGNIASTVSRLCENGLPKDACYMINYILTCIQFFFDNEFSIVPASQRGSTVEWVDNLSFVHAYALWPGQFGGLNNLQYSRLFTRNIGD

SEQ ID NO: 31 - protein, 120 aa, AVIAN AVULAVIRUS 10
VFWEGRILSQLLKNATKLCMVADHLGENSVSSCSNLSSTIARLVENGLEKDVAVLLNQVYTMTQLLFDEHYSIVCDYSNVKKLIGSQNYRNLQYATLLPGQVGGFNFLNLSRLFTRNIGD

SEQ ID NO: 32 - protein, 120 aa, SALMON AQUAPARAMYXOVIRUS
IYYDGQILPQCLKSLTRCVFWSETLVDETRAACSNIATSVAKSIENGYSPVVGYFYHNFKICQQLAISLGFSINPTLTRSVTAPLLNSTVWMTVAVFVPATVGGFNYMAMSRCFVRNIGD

SEQ ID NO: 33 - protein, 120 aa, SIMIAN RUBULAVIRUS
VFYQGRILTQALKNASKLCLTADVLGECTQASCSNAATTIMRLTENGVEKDVCYMLNVYQAIRQLCFDLYYPQYSIPGEQISQHYLKHPSIVARLVILPSQLGGLNYLSCSRLFNRNIGD

SEQ ID NO: 34 - protein, 120 aa, PORCINE RUBULAVIRUS
VFFRGRILNQALKNASKLCLIADVLGDCSQASCSNLATTVMRLAENGVEKDLCYYLNVYLTIRQVTYDIKFPQVQTYSSDIRHFYANHPHLIARLAVLPSQLGGLNYLSCSRLFNRNIGD

SEQ ID NO: 35 - protein, 120 aa, MUMPS RUBULAVIRUS
VFYKGRILTQALKNVSKMCLTADILGECSQASCSNLATTVMRLTENGVEKDLCYFLNAFMTIRQLCYDLVFPQTKSLSQDITNAYLNHPILISRLCLLPSQLGGLNFFSCSRLFNRNIGD

SEQ ID NO: 36 - protein, 120 aa, MENANGLE VIRUS
IIYNGRILNQALKNVSKLCLIADVMGESTQTSCSNLSTTIMRLTENGVEKDVCFFLNQYLTAKQLVYDLLFPMTKLCEDETTECYLNNPILISRIITIPAQLGGLNFYALTRLFNRNIGD

SEQ ID NO: 37 - protein, 120 aa, MAPUERA RUBULAVIRUS
VFFRGRILTQALKNASKLCLTADILGDCTQSSCSNLATTVMRLAENGIEKDLCIYLNYYLTVRQLTFDLTFPQYANPNTSVNTLYTNHPTWISRLALLPSQLGGLNYLSCSRLFNRNIGD

SEQ ID NO: 38 - protein, 120 aa, HUMAN RUBULAVIRUS 4
IFWRGRILSQALKNASKLCLTADILGDCTQSSCSNLATTIMRLTENGLEKDVAIRLNIFMAIRQLTFDLIFPMNTNNASNISDIYLNHPDLLARICLVPSQVGGLNYLSSSRLFNRNIGD

SEQ ID NO: 39 - protein, 120 aa, HUMAN RUBULAVIRUS 2
VFYQGRILTQALKNASKLCLTADVLGECTQASCSNSATTIMRLTENGVEKDTCYKLNIYQSIRQLTYDLIFPQYSIPGETISGIFLQHPRLISRIVLLPSQLGGLNYLACSRLFNRNIGD

SEQ ID NO: 40 - protein, 120 aa, MAMMALIAN RUBULAVIRUS 5
IFYQGRILTQALKNASKLCLTADVLGECTQSSCSNLATTVMRLTENGVEKDICFYLNIYMTIKQLSYDIIFPQVSIPGDQITLEYINNPHLVSRLALLPSQLGGLNYLSCSRLFNRNIGD

SEQ ID NO: 41 - protein, 120 aa, ACHIMOTA VIRUS 2
IFWRGRILNQALKNVSKLCLIADVLSECTQTSCSNLTTTVMRLTENGVEKDVCYYLSIFLSLKQLVFDLLFPINKTFEDTTTETYLNHPLLLSRIAILPSQLGGLNYYSTSRLFNRNVGD

SEQ ID NO: 42 - protein, 120 aa, ACHIMOTA VIRUS 1
IFFRGRILSQALKNVSKLNLIADVLSECSQTSCANLTTTVMRLTENGVEKDICYFLSIFLSVRQLVYDLLFPMTTFIEDSITTLYLNHPILIARLCVLPSQLGGLNNYSISRLFNRNIGD

SEQ ID NO: 43 - protein, 120 aa, HUMAN RESPIROVIRUS 3
PSVAALADIKRFIKANLLDRSVLYRIMNQEPGESSFLDWASDPYSCNLPQSQNITTMIKNITARNVLQDSPNPLLSGLFTNTMIEEDEELAEFLMDRKVILPRVAHDILDNSLTGIRNAI

SEQ ID NO: 44 - protein, 120 aa, BOVINE RESPIROVIRUS 3
PTVAALADIKRFIKANLLDRGVLYRIMNQEPGESSFLDWASDPYSCNLPQSQNITTMIKNITARNVLQDSPNPLLSGLFTSTMIEEDEELAEFLMDRRIILPRVAHDILDNSLTGIRNAI

SEQ ID NO: 45 - protein, 120 aa, HUMAN RESPIROVIRUS 1
PAVAALADLKRFIKAGLLDKQVLYRVMNQEPGDSSFLDWASDPYSCNLPHSQSITTIIKNVTARSVLQESPNPLLSGLFSESSSEEDLNLASFLMDRKAILPRVAHEILDNSLTGVREAI

SEQ ID NO: 46 - protein, 120 aa, PORCINE RESPIROVIRUS 1
PAVAALADLKRFIRAGLLDRQILYRVMNQESGESNFLDWASDPYSCNLPHSQSITTIIKNITARSVLQESPNPLLSGLFTYDSKEEDLNLATFLMDRKVILPRVAHEILDNSLTGIRESI

SEQ ID NO: 47 - protein, 120 aa, MURINE RESPIROVIRUS
PAVAALADLKRFIRAGLLDKQVLYRVMNQEPGDSSFLDWASDPYSCNLPHSQSITTIIKNITARSVLQESPNPLLSGLFTETSGEEDLNLASFLMDRKVILPRVAHEILGNSLTGVREAI

SEQ ID NO: 48 - protein, 120 aa, CANINE MORBILLIVIRUS
PVTSSIADLKRMIRSGLLGVEILHQVMTQYPGDSSYLDWASDPYSANLPCVQSITRLLKNITARHVLINSPNPMLKGLFHDESQDEDEALAAFLMDRKIIIPRAAHEILDNTITGAREAI

SEQ ID NO: 49 - protein, 120 aa, DOLPHIN MORBILLIVIRUS
PVTSSLADLKRMIIAGIMPEESIHQVMTQQPGDSSFLDWASDPYSANLPCVQSITRLLKNITARHVLINSPNPMLRGLFHADSHEVDESLATFLMDRHIIRPRAAHEILDNSIAVARESL

SEQ ID NO: 50 - protein, 120 aa, MEASLES MORBILLIVIRUS
PVTSSIADLKRMILASLMPEETLHQVMTQQPGDSSFLDWASDPYSANLVCVQSITRLLKNITARFVLIHSPNPMLKGLFHDDSKEEDEGLAAFLMDRHIIVPRAAHEILDHSVTGARESI

SEQ ID NO: 51 - protein, 120 aa, PDPR morbillivirus
PVTSSIADLKRMIDAGLMPEETLHQVMTQTPGESSYLDWASDPYSANLPCVQSITRLLKNITARYILISSPNPMLRGLFHEGSRDEDEELASFLMDRHIIVPRAAHEILDHSITGAREAI

SEQ ID NO: 52 - protein, 120 aa, Phocine morbillivirus
PVTSSIADLKRMIKSGLLGEEILHQVMTQNPGDSTYLDWASDPYSANLPCVQSITRLLKNITARHVLINSPNPMLKGLFHDDSQAEDEALAAFLMDRKVIIPRAAHEILDNTITGAREAI

SEQ ID NO: 53 - protein, 120 aa, Rinderpest morbillivirus
PVTSSIADLKRMIGASLMPEETLHQVMTQQPGDSSFLDWASDPYSANLVCIQSITRLLKNITARYVLINSPNPMLRGLFHNDSKEEDEQLATFLMDRSVIVPRAAHEILDHSITGAREAI

SEQ ID NO: 54 - protein, 120 aa, Cedar virus
PVTASIADVKRMILGGVLPIGILHNIMLQEPGDATYLDWCSDPYSINLKQTQSITKVIKNITARVILRNSVNPLLKGLFHEGAYEEDTELATFILDRRVILPRVGHEILNNSITGAREEI

SEQ ID NO: 55 - protein, 120 aa, Ghanaian Bat
VLVASLADIKRYISNNLLRDEIIQKIMTQVPGTASYLDWGSDPYSANIPHMQSITKMIKTITAKSILSNSKNPLLKGLFHSKSHEEDNELAAFLMDRSIIIPRAAHEIIDNSVTGAREQI

SEQ ID NO: 56 - protein, 120 aa, Hendra henipavirus
PVTASLADLKRMIEHDLMTDKVLQKVMNQEPGDASFLDWASDPYSGNLPDSQSITKTIKNITARTILRTSPNPMLKGLFHDKSFEEDLELATFLMDRRIILPRAAHEILDNSLTGAREEI

SEQ ID NO: 57 - protein, 120 aa, Mojiang virus
PITASLADLKRMINSGLMTERVLHQVMTQTPGDASFLDWASDPYSANLPSSQSITKTIKNVTARLVLSNAKNPILKGLFHNSYHEEDKSLAEFLMDRNIIMPRAAHEILDNSITGAREEI

SEQ ID NO: 58 - protein, 120 aa, Nipah henipavirus
PVTASLADLKRMIDHSIMTESVLQKVMNQEPGDASFLDWASDPYSGNLPDSQSITKTIKNITAKTILRNSPNPMLKGLFHDKSFDEDLELASFLMDRRVILPRAAHEILDNSLTGAREEI

SEQ ID NO: 59 - protein, 120 aa, Reptilian ferlavirus
PLVAALADVKRYIEIGLLTTAALTKFTTQVPGDSTELDWASDPYSANLPHSQSVTTVIKHVTARSVLSKSPNPMLEGLFHENTDEEDHDLARFLIDREIILPRVANVILDQSVTGARSAI

SEQ ID NO: 60 - protein, 120 aa, Avian avulavirus 1
PGTTAFAEVKRLEAVGLLSSSIMTNILTRPPGNGDWASLCNDPYSFNFETVASPNIVLKKHTQKVLFETCSNPLLSGVHTEDNEAEEKALAEFLLNQEVIHPRVAHAIMEASSVGRRKQI

SEQ ID NO: 61 - protein, 120 aa, Avian avulavirus 2
PVTCAIADLKWFIRSGLIPEFILKNILLRDPGDGGWSTLCADPYALNIPYTQLPTTYLKKHTQRALLSDSNNPLLAGVQLDNQYIEEEEFARFLLDRESVMPRVAHTIMESSILGKRKNI

SEQ ID NO: 62 - protein, 120 aa, Avian avulavirus 3
PLVASIADTKRYVKCGLLTPSILDSVVSITDRKGSFTTLMMDPYSINLDYIQQPETRLKRHVQKVLLQESVNPLLQGVFLETQQDEEEALAAFLLDRDIVMPRVAHAIFECTSLGRRRHI

SEQ ID NO: 63 - protein, 120 aa, Avian avulavirus 4
PLTSSWAEAKRLMEVGLLNRKFLEGILWRPPGSGTFSTLMLDPFALNIDYLRPPETIIRKHTQKVLLQDCPNPLLAGVVDPNYNQELELLAQFLLDRETVIPRAAHAIFELSVLGRKKHI

SEQ ID NO: 64 - protein, 120 aa, Avian avulavirus 5
PVSASLADVKWLIKSGIIPRYVLRNIVFRKPGEGSWITLCMDPYALNLPYVQLPTTYLKRHTQRNLLANSSNPLLRGTRIENQYEEEEELARFLLNRESVMPRVAHVVFESTVAGRRRYL

SEQ ID NO: 65 - protein, 120 aa, Avian avulavirus 6
PVSSAMADMKFYIQVGLLSPHMLKNAIFREPGDGTWTTLCADPYSLNQPYVQLPTSYLKKHTQRMLLTASTNPLLQGTRVENQYIEEERLAKFLLDRELVMPRVAHTVFETTVAGRRKHL

SEQ ID NO: 66 - protein, 120 aa, Avian avulavirus 7
PITCSLSDIKWFAKVGLMPEYILKNIVLRAPGSGTWTTLVADPYSLNITYTKLPTSYLKKHTQRTLVADSPNPLLQGVFLLNQQQEDEALCKFLLDREQVMPRAAHVIYDQSVLGRRKYL

SEQ ID NO: 67 - protein, 120 aa, Avian avulavirus 8
PVTCSLSDLKCFIAAGLLPPYVLKNVVLREPGPGTWLTLCSDPYTLNIPYTQLPTTYLKKHTQRSLLSRAVNPLLAGVQVPNQHEEEEMLARFLLDREYVMPRVAHVILETSVLGKRKQI

SEQ ID NO: 68 - protein, 120 aa, Avian avulavirus 9
PCTTALAEIKRLERAQLIPGKLIKNLLARKPSNGTWASLCNDPYSLNIETAPSPNLILKKHTQRVLFESCTNPLLQGVYSEENDTEEAELAEFLLNQEAIHPRVAHVIMEASAVGRKKQI

SEQ ID NO: 69 - protein, 120 aa, Avian avulavirus 10
PVTSSLADIKCYIMAGLLPRHILRNIMLREPSDGGWVTLCADPYALNIPYTQLPTTYLKKHTQRSLLAHSTNPLLLGVQIQTQHEEEEALAQFLLDRESVMPRVAHVVMESSILGKRKQI

SEQ ID NO: 70 - protein, 120 aa, Salmon aquaparamyxovirus
PATAAMADLKRMVKSDILTDDVLNKLMEQEPGDSTFLDWGSDPYSCNLPYSQSISAVIKNVTARSVLIDSPNPLLAGLFGEDNQEDDEDLAQFLMDRVVIMPRVCHEILDNSLTGVRESI

SEQ ID NO: 71 - protein, 120 aa, Simian rubulavirus
PLGTAVADLKRLIKCGALESWVLSNLLSRKPGTGSWATLAADPYSLNIDYLYPPTTILKRHTQNTLMEVCKNPMLKGVFTDNAREEENNLAKFLLDRDIVLPRVAHIVIEQSSVGRKKQI

SEQ ID NO: 72 - protein, 120 aa, Porcine rubulavirus
PAVSALADLKRLIRCGAIEHWVLSNILMRKPGNGNWSTLAADPYALNVDYLNPPTMFLKKHTQQVLMEQSRNPLLSGIFHENARQEENDLAQFLLDREVVMPRVAHVILAQTSCGRRKQI

SEQ ID NO: 73 - protein, 120 aa, Mumps rubulavirus
PLVSAIADVKRLIKAGCLDIWVLYNILGRRPGKGKWSTLAADPYTLNIDYLVPSTTFLKKHAQYTLMERSVNPMLRGVFSENAAEEEEELAQYLLDREVVMPRVAHVILAQSSCGRRKQI

SEQ ID NO: 74 - protein, 120 aa, Menangle virus
PLTSAIADVKRYVLAKMLPKWVLKNLLSRSPGDGDWNTLASDPYALNINYLYPPTTFLKKHTQRVLMEGSVNPMLRGIFGDSNLLEENNLSRFLLDREVVMPRVAHIIMEQSVCGRKKQI

SEQ ID NO: 75 - protein, 120 aa, Mapuera rubulavirus
PLVSAIADLKRLIISQCIEPWILGNLLGRRPGKGTWSTIAADPYALNIDYLYPPTTFLKRHTQSVLMEGSQNPLLAGIFSENAQSEENELAQFLLDREIVMPRVAHVVIAQTSCGRRKQI

SEQ ID NO: 76 - protein, 120 aa, Human rubulavirus 4
PLVSAFADIKRLIMARCIEPWVLTNIMRRPPGDGNWSTLAADPYAVNIDYLYPPTIFLKRHAQQTLMESSVNPLLNGIFNPNAKAEENNLAQFLLDRDIVLPRVAHVILAQTGCGRRKQI

SEQ ID NO: 77 - protein, 120 aa, Human rubulavirus 2
PLGTAVADLKRLIKCGALESWILYNLLARKPRKGSWATLAADPYSLNQGYLYPATTILKRHTQHTLMEICRNPMLKGVFTDNAKEEENLLAKFLLDRDIVLPRVAHIIIDQSSIGRKKQI

SEQ ID NO: 78 - protein, 120 aa, Mammalian rubulavirus 5
PVVSAVADLKRLIKSGCMDYWILYNLLGRKPGNGSWATLAADPYSINIEYQYPPTTALKRHTQQALMELSTNPMLRGIFSDNAQAEENNLARFLLDREVIFPRVAHIIIEQTSVGRRKQI

SEQ ID NO: 79 - protein, 120 aa, Achimota virus 2
PLVSAISDLKRFIDAKALPSWILKNVAARKPGKGTWSTLAADAYAINIDYLYPPTTYLKKHTQRVLMEGSVNPMLRGVFNDNSIQEEDNLAKFLLDRPVVMPRVAHIIIDQSSCGRRKQI

SEQ ID NO: 80 - protein, 120 aa, Achimota virus 1
PLTSAIADLKRFINVGILPKWILSNLLSRDPGEGTWSTLSSDPYALNINYLYPPTTYLKKHTQRVLIEHSVNPMLYGIFTDDALQEENRLSQFLLDRPIVMPRVAHIIIEQTSCGRRRQI

SEQ ID NO: 81 - protein, 50 aa, Human respirovirus 3
AGMLDTTKSLIRVGINRGGLTYSLLRKISNYDLVQYETLSRTLRLIVSDK

SEQ ID NO: 82 - protein, 50 aa, Bovine respirovirus 3
AGMLDTTKSLIRVGINRGGLTYNLLRKISNYDLVQYETLSKTLRLIVSDK

SEQ ID NO: 83 - protein, 50 aa, Human respirovirus 1
AGMLDTTKSLVRASVRRGGLSYSILRRLINYDLLQYETLTRTLRKPVKDN

SEQ ID NO: 84 - protein, 50 aa, Porcine respirovirus 1
AGMLDTTKSLVRVSIRKGGLSYNILRKLINYDLLQYETLTRTLRKVVTNN

SEQ ID NO: 85 - protein, 50 aa, Murine respirovirus
AGMLDTTKSLVRASVRKGGLSYGILRRLVNYDLLQYETLTRTLRKPVKDN

SEQ ID NO: 86 - protein, 50 aa, Canine morbillivirus
AGMLDTTKGLIRTSMKRGGLTPRIINRLSTYDYEQFRAGIRLLSGKGHDP

SEQ ID NO: 87 - protein, 50 aa, Dolphin morbillivirus
AGMLDTTKGLIRASMKRGGLTPRIITRLSNYDYDQSKMGISLLTVKKRNN

SEQ ID NO: 88 - protein, 50 aa, Measles morbillivirus
AGMLDTTKGLIRASMRKGGLTSRVITRLSNYDYEQFRAGMVLLTGRKRNV

SEQ ID NO: 89 - protein, 49 aa, PDPR morbillivirus
AGMLDTTKGLIRTSMKRGGLTPRVLARLSNYDYEQFRSGITLLTKKGQC

SEQ ID NO: 90 - protein, 49 aa, Phocine morbillivirus
AGMLDTTKGLIRASMRRGGLTPRIITRLSTYDYEQFRAGIRLLSGKRRD

SEQ ID NO: 91 - protein, 50 aa, Rinderpest morbillivirus
AGMLDTTKGLIRTSMRRGGLTTRVIARLSTYDYEQFRSGMVLLTGSKRNY

SEQ ID NO: 92 - protein, 50 aa, Cedar virus
SGLLDTTKGLIRIGIAKGGLTQRTLSRISNYDYEQFLNLMNMLKNKEQNS

SEQ ID NO: 93 - protein, 50 aa, Ghanaian Bat
AGLLDTTKGLIRSGLRKGGIQPKTVDKIANYDYQQFIVLQDLLLNTNSNP

SEQ ID NO: 94 - protein, 50 aa, Hendra henipavirus
AGLLDTTKGLIRSGLKKSGIQPKLVSRLSNHDYNQFLILNRLLSNKKRND

SEQ ID NO: 95 - protein, 50 aa, Mojiang virus
SGMLDTTKGLIRTGLSKGGIQPRLVSRLSHYDYEQFRVFNNLMRNKNMSP

SEQ ID NO: 96 - protein, 50 aa, Nipah henipavirus
AGLLDTTKGLIRSGLRKSGIQPRLVSRLSHHDYNQFLILNKLLSNRKQND

SEQ ID NO: 97 - protein, 50 aa, Reptilian ferlavirus
AGLLDTTKTLIKNTANKGGLSNRTVSKLLYHDYQQFIVFNKILRSQVDDT

SEQ ID NO: 98 - protein, 52 aa, Avian avulavirus 1
QGLVDTTNTVIKIALTRRPLGIKKLMRIINYSSMHAMLFRDDVFLSNRSNHP

SEQ ID NO: 99 - protein, 60 aa, Avian avulavirus 2
QGLIDTTPTIIKTALMRQPISRRKCDKIVNYSINYLTECHDSLLSCRTFEPRKEIIWESA

SEQ ID NO: 100 - protein, 58 aa, Avian avulavirus 3
QGLIDTTKTIIALALDTQNLSHTKREQIVTYNATYMRSLTQMLKLSRTVHKGMTRMLP

SEQ ID NO: 101 - protein, 60 aa, Avian avulavirus 4
QGLVDTTKTIIQCSLERQPLSWRKVENIITYNAQYFLGATQQIDTDSPEKQWVMPSNFKK

SEQ ID NO: 102 - protein, 60 aa, Avian avulavirus 5
QGLIDTTQTIIKTALISYPISFKKCQKISEYTANYITDFNECVYAENAKIEDPIQAWNRG

SEQ ID NO: 103 - protein, 60 aa, Avian avulavirus 6
QCLIDTTPTIIKYALHHHPISFKKSMLISSYSADYIMSFIDTIATVEYPKRDTMKLWNRC

SEQ ID NO: 104 - protein, 60 aa, Avian avulavirus 7
QGLVDTTQTIIRYALQKMPVSYKKSEKIQNYNLLYIQSLFDEVLTQNVIHSGLDTIWKRD

SEQ ID NO: 105 - protein, 60 aa, Avian avulavirus 8
QGLIDTTPTIIRTSLVNLPVSRKKCEKIINYSLNYIAECHDSLLSQICFSDNKEYLWSTS

SEQ ID NO: 106 - protein, 52 aa, Avian avulavirus 9
QGLIDTTNTIIKIALGRRPLGARRLRKINSYSSMHMLIFLDDIFLPNHPPSP

SEQ ID NO: 107 - protein, 60 aa, Avian avulavirus 10
QGLIDTTPTIIKTALMSQPISRKRCDKILQYSINYISACHDSVLSGECLTTEKRMIWDTS

SEQ ID NO: 108 - protein, 50 aa, Salmon aquaparamyxovirus
AGMLDTTKTLIRAGVRSGCLSVGMLRRLTNHDLLQFETFTKIMKQKVNDH

SEQ ID NO: 109 - protein, 54 aa, Simian rubulavirus
QGFFDTTRTIMRKSFEIKPLSSKRTLSVIECNINYLAYNFNIIHHPNPIPGYLQ

SEQ ID NO: 110 - protein, 54 aa, Porcine rubulavirus
QGFLDSTRTMIRHALQLQPLSFRKLNQVLEYNCLYLAYSLEVIEYPKEVASHLM

SEQ ID NO: 111 - protein, 54 aa, Mumps rubulavirus
QGYLDSTRTIIRYSLEVRPLSAKKLNTVIEYNLLYLSYNLEIIEKPNIVQPFLN

SEQ ID NO: 112 - protein, 56 aa, Menangle virus
QGYLDTTRTIIRYALSKQPLGFHKLNKILDYNTLFLSYNLDSILKPAVNTQIKLTT

SEQ ID NO: 113 - protein, 54 aa, Mapuera rubulavirus
QGYLDSTRTIIRHALKIQPLSTKRVITIVEYNSLYLAYVLEIIETPREVPPYLL

SEQ ID NO: 114 - protein, 54 aa, Human rubulavirus 4
QGYLDSTRTIVKLALDIKPLSFRKTNQVLDYNLNYLSYQLDIILKPRTGKDSWS

SEQ ID NO: 115 - protein, 54 aa, Human rubulavirus 2
QGFFDTTRTIMRRSFEIKPLSTKKTLSVIEYNTNYLSYNYPVILNPLPIPGYLN

SEQ ID NO: 116 - protein, 54 aa, Mammalian rubulavirus 5
QGYLDSTRSIMRKSLEIKPLSNRKLNEILDYNINYLAYNLALLKNAIEPPTYLK

SEQ ID NO: 117 - protein, 56 aa, Achimota virus 2
QGYLDTTRTIIRHALQNSPLSIKKANRIIDYNTLYLAYNLEILAAPNKGESIKLKG

SEQ ID NO: 118 - protein, 56 aa, Achimota virus 1
QGYLDTTRTIIRHALSRQPPGYKKVEKILEYNRLYLAYNQELIAKPNKKRALRAKD